(19) United States
(12) Patent Application Publication
(10) Pub. No.: US 2019/0046382 A1
(43) Pub. Date: Feb. 14, 2019

(54) METHOD OF SECURING A PATIENT ONTO AN OPERATING TABLE WHEN THE PATIENT IS IN A POSITION SUCH AS THE TRENDELENBURG POSITION AND APPARATUS THEREFOR INCLUDING A KIT

(71) Applicant: Alessio Pigazzi, Mission Viejo, CA (US)

(72) Inventor: Alessio Pigazzi, Mission Viejo, CA (US)

(21) Appl. No.: 16/164,420

(22) Filed: Oct. 18, 2018

Related U.S. Application Data
(63) Continuation-in-part of application No. 16/123,468, filed on Sep. 6, 2018.
Application No. 16/123,468 is a continuation-in-part of application No. 15/987,602, filed on May 23, 2018, now Pat. No. 10,098,800.
Application No. 15/987,602 is a continuation of application No. 15/943,245, filed on Apr. 2, 2018, now Pat. No. 10,045,902.
Application No. 15/943,245 is a continuation of application No. 15/850,126, filed on Dec. 21, 2017, now Pat. No. 9,949,883.
Application No. 15/850,126 is a continuation of application No. 14/881,274, filed on Oct. 13, 2015, now Pat. No. 9,931,262.
Application No. 14/881,274 is a continuation of application No. 13/957,778, filed on Aug. 2, 2013, now Pat. No. 9,161,876.
Application No. 13/957,778 is a continuation of application No. 13/773,290, filed on Feb. 21, 2013, now Pat. No. 8,511,314.
Application No. 13/773,290 is a continuation of application No. 13/737,552, filed on Jan. 9, 2013, now Pat. No. 8,464,720.
Application No. 13/737,552 is a continuation of application No. 13/346,852, filed on Jan. 10, 2012, now abandoned.
(60) Provisional application No. 61/654,339, filed on Jun. 1, 2012.
(60) Provisional application No. 62/720,534, filed on Aug. 21, 2018.

(51) Int. Cl. A61G 13/12; A61F 5/37; A61B 5/00; A61G 13/04

(52) U.S. Cl. CPC A61G 13/126; A61F 5/3769; A61B 5/704; A61G 13/127; A61G 13/0009; A61G 13/123; A61G 13/1225; A61G 13/121; A61G 13/04; A61G 13/1245

(57) ABSTRACT

A method of securing a patient onto an operating table when the patient is in a position such as the trendelenburg position or in a non-inclined position and apparatus therefor including a kit, which kit comprises a viscoelastic pad structured to be used to support and hold a patient on a medical procedure table during a medical procedure performed while the table, and thus the patient lying thereon, is in an inclined position, such as the Trendelenburg position or in a non-inclined position. The viscoelastic pad has characteristics which promote a minimization of pressure forces on the patient's body, as well as promote a secure cushioning and holding of the patient in a desired position on the table; said pad structure being in combination with a cleaning device configured to clean an operating instrument, in order to minimize injury to the patient.

### CONTINUING APPLICATION DATA

The present application is a continuation-in-part of Ser. No. 16/123,468, filed Sep. 6, 2018, which is a continuation-in-part of U.S. patent application Ser. No. 15/987,602, filed May 23, 2018, which is a continuation of U.S. patent application Ser. No. 15/943,245, filed on Apr. 2, 2018, which is a continuation of U.S. patent application Ser. No. 14/881,274, filed Oct. 13, 2015, which is a continuation of U.S. patent application Ser. No. 13/957,778, filed on Aug. 2, 2013, which is a continuation of U.S. patent application Ser. No. 13/773,290, filed on Feb. 21, 2013, which is a continuation of U.S. patent application Ser. No. 13/737,552, filed on Jan. 9, 2013, which is a continuation-in-part of U.S. patent application Ser. No. 13/346,852, filed on Jan. 10, 2012. U.S. patent application Ser. No. 13/773,290 also claims the benefit of U.S. Provisional Patent Application No. 61/654,339, filed on Jun. 1, 2012.

### BRIEF DESCRIPTION OF THE DRAWINGS

The present application is explained in greater detail below with reference to the accompanying drawings:

FIG. 1 shows a top view of a Trendelenburg pad of the present application comprising fasteners;

FIG. 2 shows a bottom view of the Trendelenburg pad as seen in FIG. 1;

FIGS. 3A and 3B show fasteners configured to hold the Trendelenburg pad on the operating table;

FIG. 4 shows a Trendelenburg pad of the present disclosure secured to an operating table and having a patient positioned thereon and also shows a method of use;

FIG. 5 shows a Trendelenburg pad of the present disclosure having the impression of a patient therein;

FIG. 6 shows a Trendelenburg pad of the present disclosure secured to an operating table in the Trendelenburg position and having a patient positioned thereon;

FIG. 7 shows a figure disposed on a Trendelenburg pad of the present application;

FIG. 8 is a block diagram of a robotic surgery system to perform minimally invasive robotic surgical procedures using one or more robotic surgical arms with a strap drive train;

FIG. 9 a perspective view of the robotic patient-side system of FIG. 8 with the one or more robotic surgical arms having the strap drive train;

FIG. 10 shows an example of robotic surgery; and

FIG. 11 is a representation of a table inclined at a forty-five degree angle;

FIG. 12 shows an exploded front view of an exemplification of a cleaning device and a trocar used in at least minimally invasive surgery;

FIG. 13 is a top view of an exemplification of a cleaning device attached to a trocar;

FIG. 14 shows a perspective view of an exemplification of a cleaning device attached to a trocar by a snap member;

FIG. 15 shows a perspective view of an exemplification of a cleaning device and trocar;

FIG. 16 shows an exploded view of an exemplification of a cleaning device;

FIG. 17 shows a partially transparent perspective view of an exemplification of a cleaning kit attached to a trocar or cannula;

FIG. 18 shows another view of an exemplification of the cleaning kit shown in FIG. 18;

FIG. 19 shows another view of an exemplification of the cleaning kit shown in FIG. 18;

FIG. 20 shows a view of a container of an exemplification of the cleaning kit shown in FIG. 18;

FIG. 21 shows another view of the exemplification of the cleaning kit shown in FIG. 18, with a cannula cleaner being used to dispense a cleaning medium;

FIG. 22 shows an exemplification of a cleaning kit;

FIG. 23 shows a view of a container of an exemplification of the cleaning kit shown in FIG. 22;

FIG. 24 is a perspective view of another exemplification of a scope cleaning device according to an aspect of the present disclosure;

FIG. 25 is an exploded perspective view of the scope cleaning device of FIG. 24;

FIG. 26 is a cross-sectional view of the base and the fluid reservoir of the scope cleaning device of FIG. 24;

FIG. 27 is a perspective view of the base of the scope cleaning device of FIG. 24;

FIG. 28 is a top view of the base of the scope cleaning device of FIG. 24;

FIG. 29 is a side view of the scope cleaning device of FIG. 24 connected to a trocar having a first diameter;

FIG. 30 is a side view of the scope cleaning device of FIG. 24 connected to a trocar having a second diameter;

FIG. 31 is perspective view of a device;

FIG. 32 shows a five millimeter wiping wand;

FIG. 33 shows a twelve millimeter wiping wand;

FIG. 34 shows a microfiber cloth;

FIG. 35 shows a bottle of anti-fog solution;

FIG. 36 shows a housing top 606;

FIG. 37 shows a side view of the housing top;

FIG. 38 shows an additional side view of the housing top; FIG. 39 is a perspective view of an expanded subassembly of the bottom of a housing assembly;

FIG. 40 shows a side view of the assembled bottom assembly;

FIG. 41 shows an additional side view of the housing bottom assembly;

FIG. 42 is a perspective view of an expanded subassembly of a reservoir assembly;

FIG. 43 shows a side view of the assembled reservoir subassembly;

FIG. 44 shows an additional side view of the assembled reservoir subassembly;

FIG. 45 shows an assembled reservoir subassembly;

FIG. 46 shows a cross-section of the assembled reservoir subassembly 609;

FIG. 47 shows a top view of a possible exemplification of a housing top;

FIG. 48 shows a side view of the possible exemplification of the housing top;

FIG. 49 shows a housing top;

FIG. 50 shows a cross-section of the housing top;

FIG. 51 is a perspective view of one possible exemplification of a microfiber patch;

FIG. 52 shows a front view of the microfiber patch;

FIG. 53 shows a side view of the microfiber patch;

FIG. 54 shows a first battery contact;

FIG. 55 is a top view of the first battery contact;

FIG. 56 is a side view of the first battery contact;

FIG. 57 shows a perspective view of a second battery contact;

FIG. 58 shows a side view of the second battery contact;

FIG. 59 shows a top view of the second battery contact;

FIG. 60 shows a different side view of the second battery contact;

FIG. 61 shows a perspective view of a housing bottom;

FIG. 62 shows a top view of the housing bottom;

FIG. 63 shows a side view of the housing bottom;

FIG. 64 shows a different side view of the housing bottom;

FIG. 65 shows a top view of the housing bottom;

FIG. 66 shows a cross-section of the housing bottom;

FIG. 67 shows another top view of the housing bottom;

FIG. 68 shows the bottom view of the housing bottom;

FIG. 69 shows a side view of the housing bottom;

FIG. 70 shows a front view of the housing bottom;

FIG. 71 shows a back view of the housing bottom;

FIG. 72 shows a perspective view of a reservoir;

FIG. 73 shows a top view of the reservoir;

FIG. 74 shows a side view of the reservoir;

FIG. 75 shows a cross-section of the reservoir;

FIG. 76 is a perspective view of a reservoir sponge;

FIG. 77 shows a front view of the reservoir sponge;

FIG. 78 shows a side view of the reservoir sponge;

FIG. 79 shows a perspective view of a heater;

FIG. 80 shows circuitry for the heater;

FIG. 81 is a perspective view of an inner seal;

FIG. 82 shows a side view of the inner seal;

FIG. 83 shows a top view of the inner seal;

FIG. 84 shows another side view of the inner seal;

FIG. 85 shows a cross-section of the inner seal;

FIG. 86 shows a bottom view of the inner seal;

FIG. 87 is a perspective view of an outer seal;

FIG. 88 shows a top view of the outer seal;

FIG. 89 shows a side view of the outer seal;

FIG. 90 shows a cross section of the outer seal 621; FIG. 91 is a perspective view of a reservoir installation;

FIG. 92 shows a top view of reservoir insulation in a flat pattern;

FIG. 93 shows a side view of the reservoir insulation;

FIG. 94 shows one possible side view of a reservoir retainer;

FIG. 95 shows another possible side view of the reservoir retainer;

FIG. 96 shows another possible side view of the reservoir retainer;

FIG. 97 shows a top view of the reservoir retainer;

FIG. 98 shows a bottom view of the reservoir retainer including an energy director;

FIG. 99 shows a cross section of the energy director of the reservoir retainer;

FIG. 100 shows the reservoir retainer;

FIG. 101 shows a cross section of the reservoir retainer; FIG. 102 shows a perspective view of a third battery contact;

FIG. 103 shows a side view of the third battery contact;

FIG. 104 shows another side view of the third battery contact;

FIG. 105 shows a top view of the third battery contact;

FIG. 106 shows a perspective view of a five millimeter wiping wand;

FIG. 107 shows a side view of the five millimeter wiping wand;

FIG. 108 shows an end view of the five millimeter wiping wand;

FIG. 109 shows an end view, opposite of the end shown in FIG. 108, of the five millimeter wiping wand;

FIG. 110 shows the side view of the five millimeter wiping wand as shown in FIG. 107;

FIG. 111 shows a cross section of the five millimeter wiping wand ;

FIG. 112 shows a different cross section of the five millimeter wiping wand;

FIG. 113 shows a perspective view of the components of the five millimeter wiping wand ;

FIG. 114 shows a side view of a molded handle;

FIG. 115 shows an additional side view of the molded handle;

FIG. 116 shows a top view of the molded handle;

FIG. 117 shows a perspective view of a twelve millimeter wiping wand;

FIG. 118 shows a side view of the twelve millimeter wiping wand 603;

FIG. 119 shows a cross section of the twelve millimeter wiping wand;

FIG. 120 shows a different cross section of the twelve millimeter wiping wand;

FIG. 121 shows a side view of the twelve millimeter wiping wand;

FIG. 122 shows an end view of the twelve millimeter wiping wand;

FIG. 123 shows an end view, opposite of the end shown in FIG. 122, of the twelve millimeter wiping wand;

FIG. 124 shows a perspective view of the components of the twelve millimeter wiping wand;

FIG. 125 shows a side view of a molded handle;

FIG. 126 shows an additional side view of the molded handle;

FIG. 127 shows a top view of the molded handle;

FIG. 128 shows a top view of a microfiber cloth;

FIG. 129 shows an expanded view of the device and its components;

FIG. 130 shows a side view of the completed assembly;

FIG. 131 shows another side view of the completed assembly of the device.

### DESCRIPTION OF EMBODIMENT OR EMBODIMENTS

During performance of some medical procedures, such as surgical operations, a patient may be placed on a support or operating table which is oriented horizontally, that is, perpendicularly with respect to the vertical. However, depending on the medical procedure, it may be more advantageous to orient the support or operating table, and thus the patient, at an incline with respect to the horizontal. For medical procedures relating to the lower body, such as, for example, abdominal or gynecological operations, the Trendelenburg position may be used. This position involves a patient first lying horizontally on a support table. The operating table or a portion thereof is then inclined such that the head and upper torso of the patient is at a vertically lower position than the pelvic region and/or legs of the patient, as shown, for example, in FIG. 6. In general, the support table is inclined such that the patient's head and upper torso is lowered from the horizontal anywhere in a range of approximately five, ten, or fifteen degrees to approximately twenty, thirty, or forty-five degrees or more, in a steep Trendelenburg position, in one degree increments or fractions of one degree increments. In addition to the positive Trendelenburg position, there is a negative or reverse Trendelenburg position, where the head and upper torso is at a vertically higher position than the pelvic region and/or legs of the patient. As used herein, the phrase Trendelenburg position should be understood as referring to both positions.

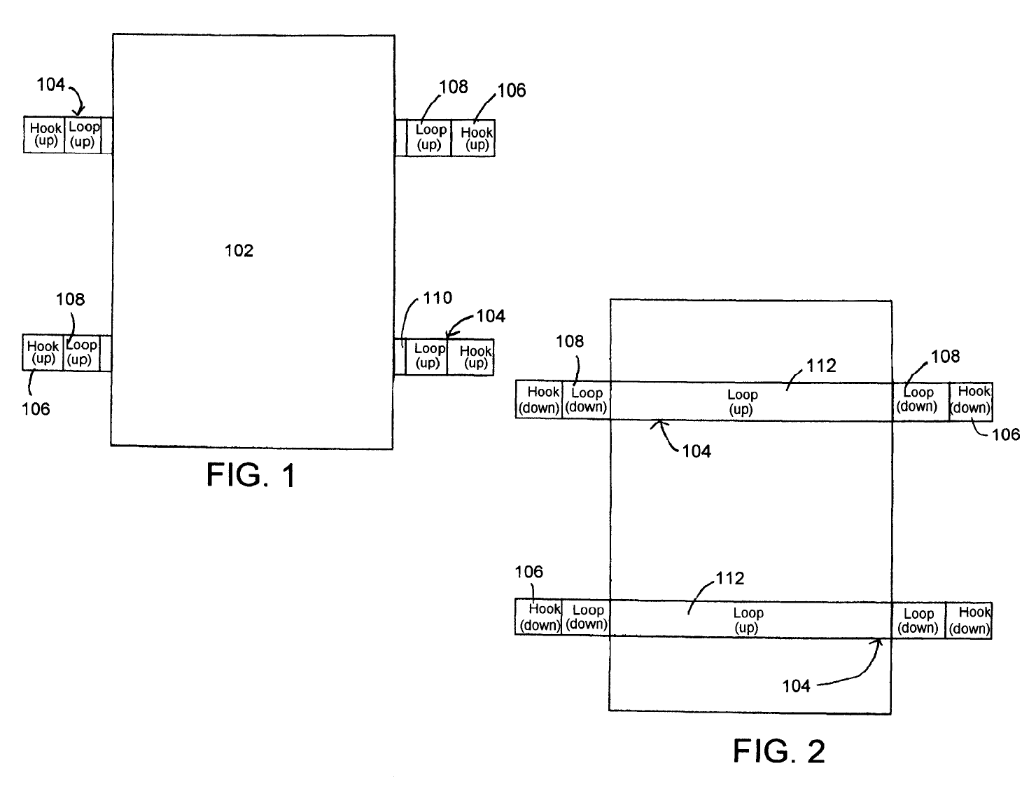

The present application discloses the use of a viscoelastic foam pad, such as shown in FIGS. 1, to assist in holding the patient in a desired position on an inclined support table, such as in the Trendelenburg position, and to assist in minimizing sliding, shifting, or similar undesirable movements of the patient on the support table, which movements could be disruptive to a medical procedure being performed on the patient. The viscoelastic foam also cushions and supports the patient while promoting a distribution of pressure forces across the patients body to reduce and/or minimize damage to nerves and/or tissue from concentrated pressure forces. For example, the viscoelastic foam will minimize or can eliminate brachial plexopathy, including pain, decreased movement, or decreased sensation in the arm and shoulder by minimizing pressure to a patient's neck, arms, and/or shoulders, and thereby minimizing or reducing a nerve event. In at least one possible embodiment, the viscoelastic foam has characteristics which are selected to promote the preceding desired effects, which will be discussed in the following paragraph. All of the characteristics discussed in the following paragraph are according to at least one possible embodiment, and it should be understood that any one or more of the characteristics could be combined with any one or more of the characteristics according to at least one possible embodiment, and any ranges disclosed in the following paragraph are to be understood as including any value therein, including increments of tenths and hundredths of the particular range.

The rate of recovery, that is, the time required for a viscoelastic foam to return to its starting shape, is in the range of approximately 2-10 seconds for approximately 50 percent to 80 percent recovery after deformation caused by placing an adult torso on an approximately one inch thick layer of viscoelastic foam. The rate of recovery is in the range of approximately 6-15 seconds for approximately 80 percent to 90 percent recovery after deformation caused by placing an adult torso on an approximately one inch thick layer of viscoelastic foam. The rate of recovery is in the range of approximately 10-35 seconds for 100 percent recovery after deformation caused by placing an adult torso on an approximately one inch thick layer of viscoelastic foam. The ball rebound of the viscoelastic foam is in the range of less than or substantially less than approximately one percent to approximately one percent or several percent, or is in the range of or approximately in the range of 0.1 percent to 1.9 percent, or up to 3 percent or 5 percent, and as much as several percent. The compression set (the residual compression of the foam after twenty-two hours at seventy degrees Celsius) of the viscoelastic foam, for a 25 percent compression of the foam, is in the range of less than one percent or tenths of a percent, such as, for example, less than 0.4 percent or 0.3 percent, to several percent. The compression set of the viscoelastic foam, for a fifty percent compression of the foam, is in the range of less than one percent or tenths of a percent, such as, for example, less than 0.5 percent, to several percent. The indentation force deflection of the viscoelastic foam, at a 25 percent deflection, is in the range of several pounds of force to tens of pounds of force, such as in the range of approximately 10 to approximately 15 pounds, or in the range of approximately 7 to approximately 18 pounds, or is in the range of approximately 12 pounds, such as, for example, 11.7, 11.8, 11.9, 12.0, 12.1, 12.2, 12.3, and 12.4 pounds. The coefficient of static friction between the viscoelastic foam and the surface of a support table is greater than 0.2, or is in the range of approximately 0.2 or 0.5 to approximately 0.7 or 1.0. The viscoelastic foam is designed such that the patient may shift on the viscoelastic foam less than one inch, or up to approximately an inch, or in the range of approximately one inch to approximately three inches. The airflow in or through the viscoelastic foam is in the range of tenths of a cubic foot per minute to several cubic feet per minute, such as, for example, approximately 0.1 or 0.3 cubic foot per minute to approximately 1.0 or 3.0 cubic feet per minute, or is in the range of approximately 0.53 cubic feet per minute, plus or minus 0.3 cubic feet per minute. The thickness of the viscoelastic foam is in the range of from three-fourths of an inch to one inch, or to approximately one inch, or to one and a half inches, or to three inches or greater, which thickness is selected to minimize and/or prevent bottoming out on the operating table of one or more of the portions of the body of a patient lying on the viscoelastic foam pad, depending on the weight and/or size of the patient. The tensile strength (at 25 percent deflection) of the viscoelastic foam is in the range of at least approximately 5 pounds per square inch (PSI) or approximately 8 PSI to approximately 12 PSI or approximately 15 PSI, or is in the range of approximately 10 PSI. The tear strength (in a twenty inches per minute test) of the viscoelastic foam is in the range of approximately one to approximately two or three pounds of force per inch, or in the range of approximately 1.5 pounds of force per inch. The elongation (in a twenty inches per minute test) of the viscoelastic foam is in the range of between 125 and 250 percent, or is in the range of approximately 172 percent, plus or minus 25 percent. The nominal density of the viscoelastic foam is in a range of approximately 100 kilograms per cubic meter, or is in the range of approximately 75 or 83 kilograms per cubic meter to approximately 103 or 110 kilograms per cubic meter, such as, for example, 93.1 kilograms per cubic meter. The flammability of the viscoelastic foam should pass various tests by CAL, FMVSS and FAR, and the viscoelastic foam should pass the European Union's Restriction of Hazardous Substances (EU RoHS) standards.

According to at least one possible embodiment, the viscoelastic foam pad can be of varying lengths, and can extend from either the feet, lower legs, thighs, or buttocks of a patient to either the shoulders, head, or top of the head of a patient. According to at least one possible embodiment, the viscoelastic foam pad or substantial portions thereof are pink in color, the straps for securing the patient to the pad are purple in color, and the straps for securing the pad to the table or surface are white.

As shown in FIG. 1, a Trendelenburg pad 102 may comprise fasteners 104 extending beyond longitudinal sides of the Trendelenburg pad 102. Each end of the fasteners 104 may comprise a hook and loop fastener, such as a Velcro® fastener, configured and disposed to attach the Trendelenburg pad 102 to operating table rails or other adjacent structures, depending on the support structure on which the patient is positioned, which may or may not be an operating bed. For example, each end of a fastener 104 may comprise a loop portion 108 and a hook portion 106, extending beyond the loop portion 108. Additionally, one or more portions of the fasteners 104 extending beyond the Trendelenburg pad 102 may comprise a label 110 indicating the orientation that the Trendelenburg pad 102 is to have with an operating table. For example, the label 110 may state “this side up.”

In at least one possible embodiment of the present application, the Trendelenburg pad 102 may be a disposable pad and/or single-use pad.

FIG. 2 shows a bottom view of a Trendelenburg pad 102 comprising fasteners 104 extending latitudinally therewith. A central portion 112 of the fasteners 104 may be configured to be fastened with the Trendelenburg pad 102. For example, the central portion 112 may comprise hooks configured and disposed to fasten with loops on the Trendelenburg pad 102. The Trendelenburg pad 102 may be configured to fasten with two or more fasteners 104. The fasteners may be Velcro®.

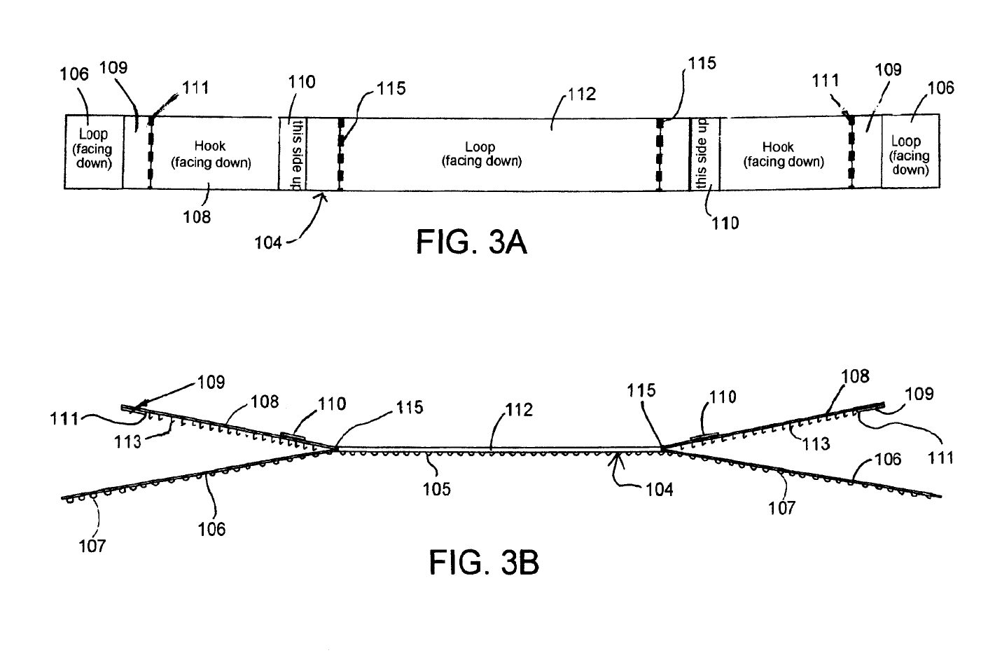

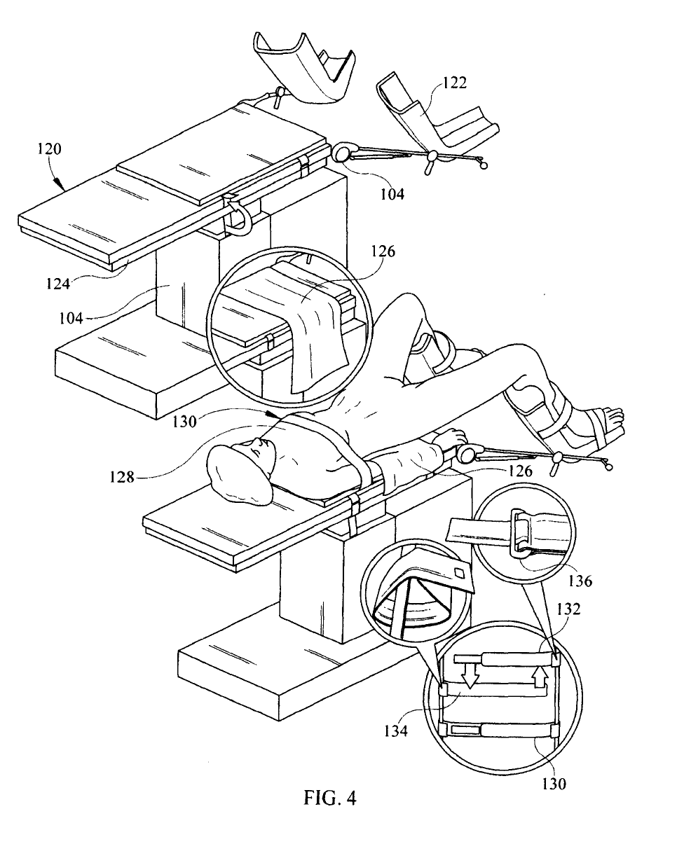

FIGS. 3A and 3B show a top view and a side view of a fastener 104 respectively. A central portion 112 of fastener 104 may comprise loops on a side configured to fasten with hooks on a Trendelenburg pad 102 as shown in FIG. 2. A weld 115 may secure a hook portion 108 and a loop portion 106 to the central portion 112 wherein the ends of fastener 104 comprises a hook portion 108 and a loop portion 106. It is to be understood that either portion 108 or 106 may comprise hooks and the other of 108 or 106 may comprise loops. A label 110 may be secured to an upper side of hook portion 108. An end of the hook portion 108 may be folded and welded at a weld point 111 to form a tab 109. Hooks 113 may extend from a lower surface of the hook portion 108. Loops 107 may extend from a lower surface of the loop portion 106.

FIG. 4 shows a use of Trendelenburg pad 102. For example, Trendelenburg pad 102 may be used by:

1. Placing a surgical operating table 120, having surgical operating table rails 124, in a Lithotomy Position.

2. Placing a longitudinal distal end of the Trendelenburg pad 102 on a longitudinal distal end of the surgical operating table 120.

3. Latitudinally centering the Trendelenburg pad 102 with the surgical operating table 120.

4. Positioning straps 104, extending from longitudinal edges of the Trendelenburg pad 102, down away from where a patient 128 will lie.

5. Attaching the straps 104 to the surgical operating table rails 124.

6. Laying a lift sheet 126 over the Trendelenburg pad 102.

7. Laying a patient 128 on the Trendelenburg pad 102 by positioning the patient 128 so that the patient's shoulders do not extend past edges of the Trendelenburg pad 102.

8. Lifting the lift sheet 126 thereby lifting the patient 128 up and off the Trendelenburg pad 102 to reposition the patient 128 as needed.

9. Positioning the patient's arms as needed.

10. Attaching body straps 130 around the patient and the surgical operating table.

11. Securing the patient's legs in stirrups 122.

12. Placing the surgical operating table in the Trendelenburg position.

In at least one possible embodiment of the present application, a patient may be disposed on a Trendelenburg pad 102 such that the patient's skin contacts the Trendelenburg pad 102, for example that the patient's skin contacts the Trendelenburg pad 102 in the patient's sacrum and scapula areas of the body and held by the Trendelenburg pad 102. The lift sheet 126 may be used to lift and reposition the patient without dragging the patient across the Trendelenburg pad.

Also shown in FIG. 4 are body straps 130. The body straps 130 may comprise a hook strap 132 and a loop strap 134, such as a Velcro® strap. Each hook strap 132 and each loop strap 134 may comprise a clasp at one end. A hook strap 132 may be secured to a operating table rail 124 by looping therearound and extending through the clasp. A loop strap 134 may be secured to the other operating table rail 124 by looping therearound and extending through the clasp, or secured by extending the hook strap 132 around or to some other portion of the operating table. The hook strap 132 and the loop strap 134 may be fastened about a patient 128, holding the patient 128 to the Trendelenburg pad 102. It is to be understood that body straps 130 are optional as the Trendelenburg pad 102 may provide adequate support of the patient 128 a on surgical operating table 120.

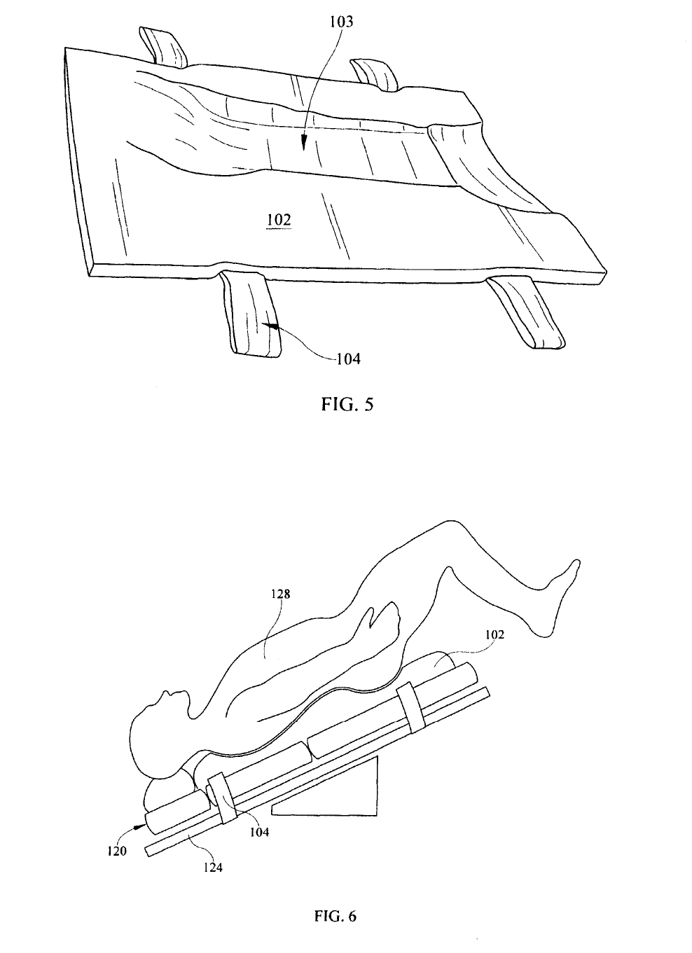

FIG. 5 shows a Trendelenburg pad 102 having an impression or residual compression 130 of a patient therein. The Trendelenburg pad 102 comprises viscoelastic foam which conforms to the patient's body and shapes itself to support the body. This shape minimizes pressure points on the body and helps to hold the body on the operating table.

The holding quality or ability between the Trendelenburg Pad and a patient will be a combination of the coefficient of friction between the patient and the viscoelastic foam and the holding ability of the impression made by the patient in the viscoelastic foam, because the Trendelenburg pad is made of a viscoelastic foam in which an impression is formed and held in the foam at least for a time after the patient is moved from one position to another or from the operating table (see FIG. 5). The equivalent total frictional characteristic, which includes the friction due to the coefficient of friction and the friction or friction-like force due to the viscoelastic foam deformation, may be equal to or in excess of one. That is to say, the normal, perpendicular force onto the viscoelastic pad from the body of the patient and component of the force of gravity on the patient parallel to the surface of the operating table at forty-five degrees are equal, which means that the equivalent total frictional characteristic will be equal to or greater than the normal, perpendicular force. For an angle of forty-five degrees, the equivalent total frictional characteristic is one, which is [sin 45°/cos 45°]. Stated more generally, the equivalent total frictional characteristic would be greater than the sine of the angle of inclination from the horizontal over the cosine of the angle of inclination from the horizontal. Since the angle can vary in one degree or smaller increments, the range of values of the equivalent total frictional characteristic could vary between any of those angles so as to be at least or greater than the sine over cosine values of each of these angles of inclination at least within the range of greater than zero degrees to forty-five degrees and also somewhat over forty-five degrees and even greater. The holding capabilities are a combination of the holding ability of the pad, the body straps, and the coefficient of friction of the pad with respect to the patient and the table, between the pad and the sheet between the pad and the patient, and between the sheet and the patient, for all described angles.

FIG. 6 shows a Trendelenburg pad 102 of the present application secured to a surgical operating table 120 with fasteners 104. A patient 128 is positioned on the Trendelenburg pad 102, and the surgical operating table 120 is in the Trendelenburg position. Also shown in FIG. 6 is an aspect of the Trendelenburg pad 102 wherein body straps 130 are not needed to hold a patient 128 on the surgical operating table 120 in the Trendelenburg position.

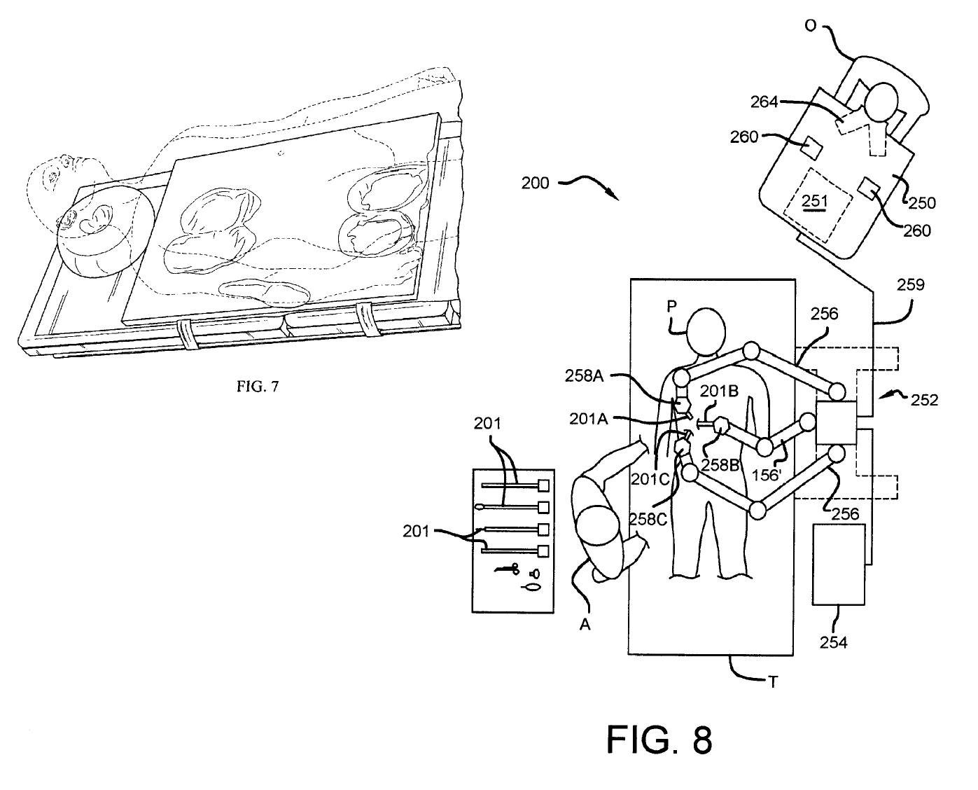

FIG. 7 is a computer illustration of a person disposed on a Trendelenburg pad of one possible embodiment of the present application. The figure of the person is transparent or see-through, permitting a view of the impression on the Trendelenburg pad made by the figure of the person. The impression on the Trendelenburg pad may provide the primary holding for holding a person on the operating table depending on the angle of the patient on the table.

Referring now to FIG. 8, a block diagram of a robotic surgery system 200 is illustrated to perform minimally invasive robotic surgical procedures using one or more robotic arms with strap drive. Robotic surgery generally involves the use of a robot manipulator that has multiple robotic manipulator arms. One or more of the robotic manipulator arms often support a surgical tool which may be articulated (such as jaws, scissors, graspers, needle holders, micro dissectors, staple appliers, tackers, suction/irrigation tools, clip appliers, or the like) or non-articulated (such as cutting blades, cautery probes, irrigators, catheters, suction orifices, or the like). At least one of the robotic manipulator arms (e.g., the center robotic manipulator arm 258B) is used to support a stereo or three dimensional surgical image capture device 210 such as a stereo endoscope (which may be any of a variety of structures such as a stereo laparoscope, arthroscope, hysteroscope, or the like), or, optionally, some other stereo imaging modality (such as ultrasound, fluoroscopy, magnetic resonance imaging, or the like). Robotic surgery may be used to perform a wide variety of surgical procedures, including but not limited to open surgery, neurosurgical procedures (such as stereotaxy), endoscopic procedures (such as laparoscopy, arthroscopy, thoracoscopy), and the like.

A user or operator O (generally a surgeon) performs a minimally invasive surgical procedure on patient P by manipulating control input devices 260 at a master control console 250. A computer 251 of the console 250 directs movement of robotically controlled endoscopic surgical instruments 201A-201C by means of one or more control cables 259, effecting movement of the instruments using a robotic patient-side system 252 (also referred to as a patient-side cart). The robotic patient-side system 252 has one or more robotic arms 258. In one possible embodiment of the Da Vinci Surgical System, the one or more robotic arms 258 have a strap drive system. Typically, the robotic patient-side system 252 includes at least three robotic manipulator arms 258A-258C supported by linkages 256, 256′, with a central robotic arm 258B supporting an endoscopic camera 201 B and the robotic arms 258A, 258C to left and right of center supporting tissue manipulation tools 201A and 201C.

Generally, the robotic patient-side system 252 includes a positioning portion and a driven portion. The positioning portion of the robotic patient-side system 252 remains in a fixed configuration during surgery while manipulating tissue. The driven portion of the robotic patient-side system 252 is actively articulated under the direction of the operator O generating control signals at the surgeon's console 250 during surgery. The actively driven portion of the robotic patient-side system 252 is generally referred to herein as the robotic arms or alternatively to robotic surgical manipulators. The positioning portion of the robotic patient-side system 252 that is in a fixed configuration during surgery may be referred to as “set up arms” 256, 256′ with positioning linkage and/or “set-up joints” (SUJ). In an alternate embodiment of the Da Vinci Surgical System, the robotic patient-side system 252 may be replaced by set up arms that couple at one end to left and right sides of the operating table T. The three robotic manipulator arms 258A-258C may then be coupled to the opposite end of the set-up arms to ground to the table T.

For convenience in terminology, manipulators such as robotic surgical arms 258A and 258C actuating the tissue affecting surgical tools 201A and 201C are generally referred to herein as a PSM (patient-side manipulators), and a robotic surgical arm 258B controlling an image capture or data acquisition device, such as the endoscopic camera 201 B, is generally referred to herein as a ECM (endoscope-camera manipulator), it being noted that such telesurgical robotic manipulators may optionally actuate, maneuver and control a wide variety of instruments, tools and devices useful in surgery. The surgical tools 201A, 201C and endoscopic camera 201 B may be generally referred to herein as tools or instruments 201.

An assistant A may assist in pre-positioning of the robotic patient-side system 252 relative to patient P as well as swapping tools or instruments 201 for alternative tool structures, and the like, while viewing the internal surgical site via an assistant's display 254. With the embodiments of the Da Vinci Surgical System, the assistant A may also swap in and out the robotic surgical arms 258A and 258C, as well as the robotic surgical arm 258B, in case one is defective or failing. In other cases, a robotic surgical arm may be swapped out for maintenance, adjustments, or cleaning and then swapped back in by one or more service persons.

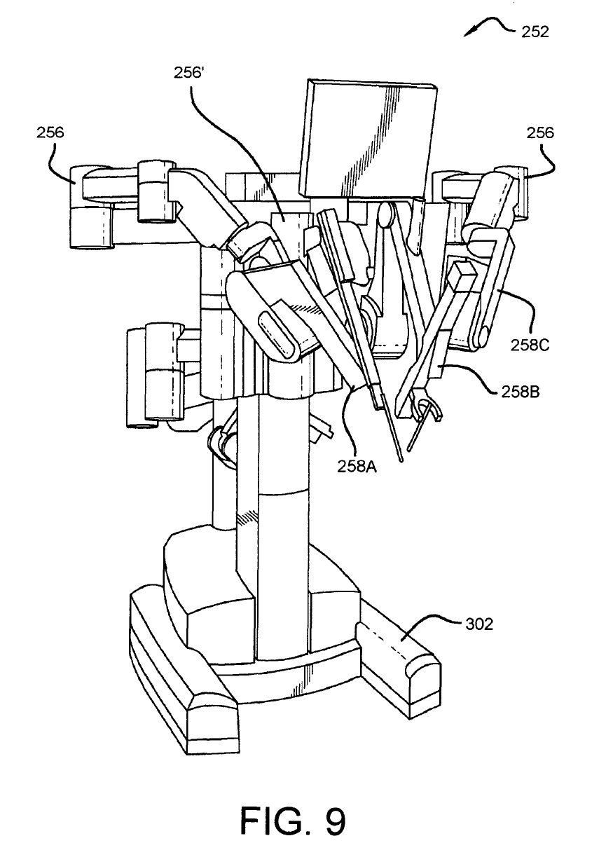

Referring now to FIG. 9, a perspective view of the robotic patient-side system 252 is illustrated. The robotic patient-side system 252 may have one or more robotic surgical arms (a.k.a., robotic surgical manipulators) 258A-258C with a strap drive system. The robotic arms 258A, 258C are for coupling to robotic surgical tools 201A, 201C. The robotic arm 258B is for coupling to an endoscopic camera 201 B. Generally, the surgical robotic arms 258A-258C may be referred to as a surgical robotic arm or a robotic surgical arm 258.

The robotic patient-side system 252 further includes a base 302 from which the robotic surgical instruments 201 may be supported. In at least one possible embodiment of the Da Vinci Surgical System, the robotic surgical instruments 201 are each supported by the positioning linkage 256 and the surgical robotic arms 258. The linkage structures may optionally be covered by protective covers or not to minimize the inertia that is manipulated by the servomechanism and the overall weight of robotic patient-side system 252.

The robotic patient-side system 252 generally has dimensions suitable for transporting between operating rooms. It typically can fit through standard operating room doors and onto standard hospital elevators. The robotic patient-side system 252 may have a weight and a wheel (or other transportation) system that allows the cart to be positioned adjacent to an operating table by a single attendant. The robotic patient-side system 252 may be sufficiently stable during transport to avoid tipping and to easily withstand overturning moments that may be imposed at the ends of the robotic arms during use.

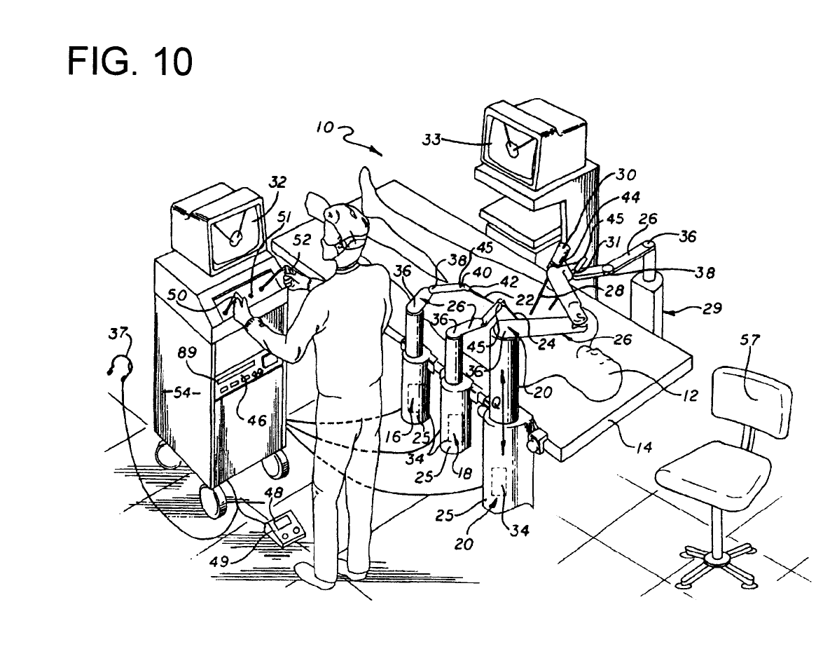

FIG. 10 shows an example of a robotic surgery system 10, in which at least one possible embodiment of the Trendelenburg pad could be utilized. In robotic surgery, the consistency of location of the patient is paramount because a shift in position may interfere with the relative positioning of the instruments used in surgery. Due to the ability of the Trendelenburg pad to hold or assist in holding a patient in a desired position, the Trendelenburg pad could be used in robotic surgery. The system 10 may be used to perform a procedure on a patient 12 that is typically lying on an operating table 14. Mounted to the operating table 14 is a first articulate arm 16, a second articulate arm 18 and a third articulate arm 20. The articulate arms 16-20 are mounted to the table so that the arms are in a plane proximate the patient.

The articulate arms 16, 18 and 20 may each comprise a base housing 25 and a robotic arm assembly 26 extending from the base housing 25. Surgical instruments 22 and 24 may be removably coupled at the end of each robotic arm assembly 26 of the first and second articulate arms 16, 18. Each of the instruments 22, 24 may be coupled to a corresponding robotic arm assembly 26.

The third articulate arm 20 may additionally comprise a base housing 25 and a robotic arm assembly 26, and has a first endoscope 28 that is attached to the robotic arm assembly 26. The base housing 25 and robotic arm assemblies 26 of each of the articulate arms 16, 18, and 20 are substantially similar. Additionally, a fourth robotic arm 29 may be included in the system 10. The fourth arm 29 may hold a second endoscope 31.

The instruments 22, 24 and endoscope 28 may be inserted through incisions cut into and through the skin of the patient 12. The first endoscope 28 may comprise a camera 30 that may be coupled to a monitor 32. The monitor 32 may be configured to display images of the internal organs of the patient 12. Additionally, the second endoscope 31 may be inserted through a corresponding incision made in the patients skin. The second endoscope 31 may be used to provide a wide field of view as depicted in FIG. 10. The second endoscope 31 may be mounted to the fourth robotic arm 29 and may be coupled to a second monitor 33.

Each robotic arm assembly 26 may comprise a base motor 34 which moves the arm assembly 26 in a linear fashion, relative to the base housing 25, as indicated by arrow Q. Each robotic arm assembly 26 may also comprise a first rotary motor 36 and a second rotary motor 38. Each of the robotic arm assemblies 26 may also comprise a pair of passive joints 40 and 42. The passive joints 40, 42 may be disposed orthogonal to each other to provide pivotal movement of the instruments 22, 24 or the endoscopes 28, 31 attached to a corresponding robotic arm assembly 26. The joint angle may be controlled to a particular value using a feedback control loop. The robotic arm assemblies 26 may also comprise a coupling mechanism 45 to couple the instruments 22 and 24, or endoscope 28, 31 thereto. Additionally, each of the robotic arm assemblies 26 may comprise a motor driven worm gear 44 being configured to rotate the instrument 22, 24 or endoscope 28, 31 attached thereto about its longitudinal axis.

The first, second, and third articulate arms 16, 18, 20, as well as the fourth arm 29, may be coupled to a controller 46 which may control the movement of the arms. The arms may be coupled to the controller 46 via wiring, cabling, or via a transmitter/receiver system such that control signals may be passed from the controller 46 to each of the articulate. Each arm 16, 18, 20 and 29 may be electrically connected to the controller 46 via electrical cabling 47.

The controller 46 may be connected to an input device 48 such as a foot pedal, hand controller, or voice recognition unit, to control the position of the endoscope 28 or the second endoscope. To effectuate voice recognition, a microphone 37 is included in the system 10. The controller 46 receives the input signals from the input device 48 and moves the endoscope 28 and robotic arm assembly 26 of the third articulate arm 20 in accordance with the input commands of the surgeon.

The movement and positioning of instruments 22, 24 attached to the first and second articulate arms 16 and 18 is controlled by a surgeon at a pair of master handles 50 and 52. Additionally, a switch 51 may be included in the system 10. The switch 51 may be used by the surgeon to allow positioning of the fourth arm 29. The handles 50 and 52 may be mounted to a portable cabinet 54. A television monitor 56 may be placed onto the cabinet 54 and coupled to the endoscope 28 so that the surgeon can readily view the internal organs of the patient 12. To accommodate a seated position, a chair 57 may be provided with the system.

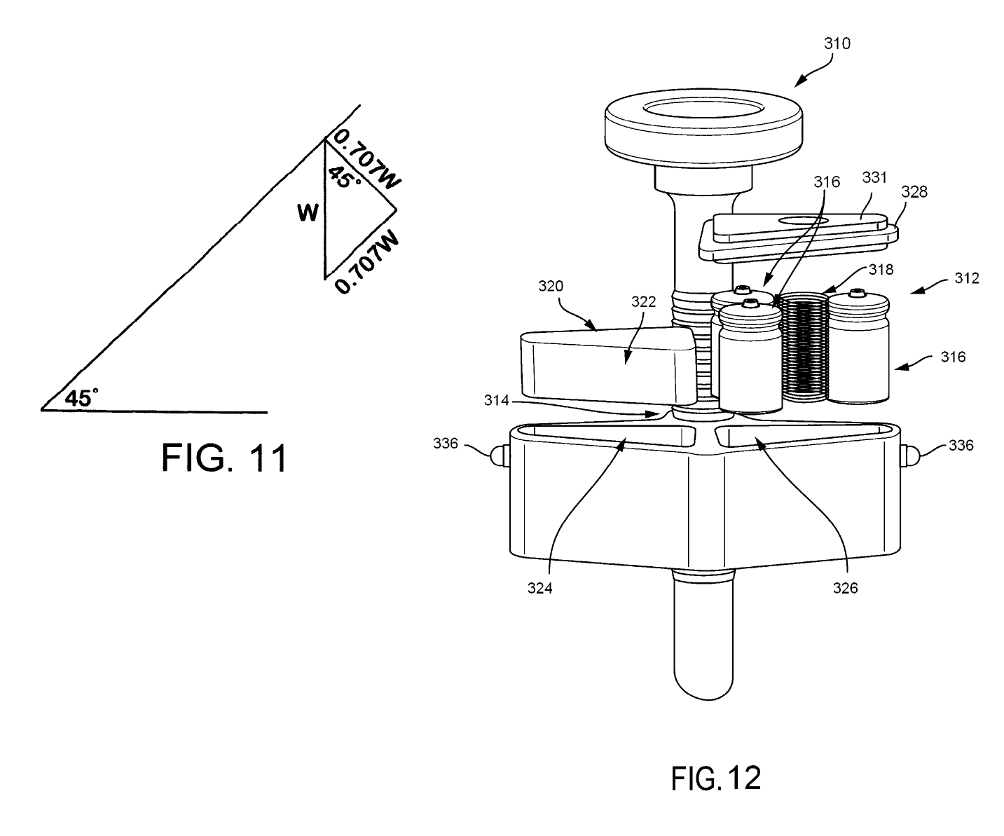

FIG. 11 is a representation of a table inclined at a forty-five degree angle. W is the weight of a patient. The normal force perpendicular to the surface of the operating table is 0.707 W. The force along the table is 0.707 W. The equivalent coefficient of friction including the actual coefficient of friction and holding ability of the Trendelenburg pad and the fasteners 130 of the present application to hold a patient is 1.00.

The positioning arrangement and arrangements in the present application could be used in gynecological procedures, colorectal procedures, urological procedures, laparoscopic procedures, and robotic procedures to name some procedures to use this pad in the Trendelenburg or the reverse Trendelenburg position. The positioning arrangement and arrangements, and embodiments of the Trendelenburg pad and kit, in the present application could also be used in all types of medical procedures in which it may be desirable to place a patient in a secure and/or reduced pressure point and/or comfortable position, which medical procedures include surgical procedures and non-surgical procedures, such as non-surgical examinations and/or treatments.

The viscoelastic foam of the present application may be a polyurethane foam made by mixing polyhydroxy polyol with toluene di-isocyanate or other and different methods as are known in the art. For example, Toluene di-isocyanate may be used in combination with polyester polyols and polyether to make viscoelastic foam.

In at least one other aspect of the present disclosure, the Trendelenburg pad 102 is about twenty inches wide, about thirty inches long, and about one inch thick. In one possible embodiment of the present application, the thickness of the pad may be in the range of approximately three-quarters of an inch to approximately one and one-half inches. In that range, the thickness may increase or decrease in increments of 1/32 of an inch. In one possible embodiment of the present application, the width of the pad may be in range of approximately twenty inches to approximately twenty-eight inches. In that range, the width may increase or decrease in increments of one-fourth of an inch or less. In one possible embodiment of the present application, the length of the pad may be in the range of approximately thirty inches to approximately forty inches. In that range, the length may increase or decrease in increments of one-fourth of an inch or less. The fasteners 104 may be Velcro® straps and may be about two inches wide. The fasteners 104 may be secured to the Trendelenburg pad 102 by welding, adhesive, hook and loop fastening, or by other means as is known in the art.

The present application relates to a cleaning device for cleaning a scope, laparoscope, or microscope used in surgery or other medical procedures, and a method of using the device during surgical or other medical procedures. The present application further relates to a cleaning kit for cleaning a trocar and a scope, laparoscope, or microscope, used in surgery or other medical procedures, and a method of using the kit during surgical or other medical procedures.

During minimally invasive surgeries, such as laparoscopic surgery, trocars are used to permit access into a patient's body. Trocars are medical devices or instruments that comprise hollow tubes or hollow portions. During surgery, such as laparoscopic surgery, a trocar may be inserted through an incision into a patient's body, such as into the abdominal or pelvic cavity of a patient. The trocar acts as a passageway for other surgical tools or devices, such as scopes, laparoscopes, microscopes, knives, graspers, scissors, staplers, and more, to enter the body for use during surgery.

During such surgical procedures, the lenses of viewing devices inserted into the body through the trocar, such as scopes, laparoscopes, and/or microscopes, may become clouded or the view therethrough may become partially or completely obscured. The view can be obscured, either partially or completely, if the lenses are smeared with bodily fluids or the like, or if the lenses become clouded or fogged over due to the humidity within the body of the patient. In addition, the interior of the trocars should also be kept clean.

An object of the present application may be to provide a cleaning device for a trocar and surgical scopes, which cleaning device may be utilized during surgery. The cleaning device also may be used to defog lenses or optics of a surgical scope.

When minimally invasive surgeries are performed using trocars, the scopes and/or microscopes used during surgery, which often are inserted into the body of the patient, may become smeared with bodily fluids or the like. This may cause the lens of the scope or microscope to become obscured or clouded because of smearing and/or the humidity or moisture within the body of the patient. Embodiments of the cleaning device and method described herein address these problems. There may be other applications for the scope or microscope which create the same problems as with minimally invasive surgery, and embodiments of the device and method described below may be appropriate to address those problems as well.

An exemplification of the cleaning device according to the present application may be configured for cleaning a surgical scope during minimally invasive surgery. The cleaning device may comprise a casing which houses a sponge and a heating element, and an attachment element connected to an outer surface of the casing and configured to removably attach the casing to the trocar, wherein the casing is configured to allow a lens of the surgical scope to access the sponge and the heating element. In an embodiment, the casing may comprise a first depression formed in an upper surface of the casing and configured to receive the sponge therein, and a second depression formed in an upper surface of the casing and configured to receive the heating element therein. In an embodiment, the sponge may be impregnated with a cleaning medium. In an embodiment, the cleaning device may include a snap member that is configured to removably attach the casing to the trocar.

In an embodiment, the heating element may be comprised of a heating coil and at least one battery operatively connected to the heating coil, wherein the heating coil is configured to warm the lens of the surgical scope when the surgical scope is positioned in proximity to the heating coil. In an embodiment, the cleaning device may further comprise at least one light operatively connected to the at least one battery.

In an embodiment, the heating element may be comprised of a chemical pack.

An embodiment of the cleaning device described herein may comprise a cover and a wiping element, wherein the cover is disposed over the second depression, wherein the cover comprises a first hole, wherein a wiping element comprises a second hole, and wherein the wiping element is disposed over the cover so that the first hole and the second hole are oriented to allow the surgical scope to access the heating element. In an embodiment, the wiping element may be comprised of a microfiber material.

Another embodiment disclosed herein is a cleaning kit for use during minimally invasive surgery, comprising a container housing a sponge and configured to removably attach to the trocar; a cover comprising a scope access hole configured to permit a surgical scope to access the sponge; the cover positioned on an upper surface of the container; and a cannula cleaner comprising an elongated body having a first end having a cleaning tip disposed thereon and a second end, the cannula cleaner configured to be removably supported by at least one of the container and cover when not in use, wherein the cannula cleaner is configured for insertion in a cannula of a trocar and the cleaning tip is configured to contact the cannula when the cannula cleaner is inserted into the cannula. An embodiment of the cleaning kit may include a cannula cleaner wherein the elongated body further comprises a solution container configured to receive a cleaning medium, and a valve disposed on the second end of the elongated body, wherein the valve is configured to dispense the cleaning medium during surgery. In an embodiment, the cleaning kit may comprise at least one wiping element disposed on the cover, wherein the at least one wiping element is configured to allow the surgical scope to access the sponge through the scope access hole. In an embodiment, the cleaning kit may further comprise a snap member configured to attach the container to the trocar.

In an embodiment, the cleaning kit may further comprise a heater disposed in the container, wherein the heater is configured to warm the surgical scope inserted through the scope access hole. In an embodiment, the heater is configured to border a substantial portion of a perimeter of the sponge. In an embodiment, the heater comprises a heating coil and at least one battery operatively connected to the heating coil, and the heating coil is configured to warm the lens of the surgical scope during surgery. In an embodiment, the heater may comprise a chemical pack.

In an embodiment, the cleaning kit may further comprise one or more lights operatively connected to at least one battery.

Another embodiment is directed to a method of cleaning a surgical scope during minimally invasive surgery comprising inserting a trocar into a patient, attaching a cleaning device to the trocar, heating a heating element of the cleaning device to a desired temperature, inserting the surgical scope into the trocar, removing the surgical scope from the trocar, wiping the lens of the surgical scope on the wiping element, and heating the lens of the surgical scope with the heating element. In an embodiment, this method further may comprise wiping the lens on the sponge.

One feature or aspect of an embodiment is using a cleaning device for cleaning laparoscopes used in a medical procedure, the cleaning device comprising a body with a snap device attached to the body, which snap device is configured to attach to a trocar. The body comprises an isosceles triangular shape with a longer side connecting the equal sides of the isosceles triangle with the snap device being attached to the middle of said longer side of the isosceles triangle, and the body comprising two hollowed out portions, one of said hollowed out portions being disposed on each side of said snap arrangement. A first hollowed out portion being configured to house a sponge impregnated with a cleaning solution, which cleaning solution is configured to clean off the tip portion of a laparoscope, where a lens is disposed, which tip portion is configured to be inserted into the trocar and into the body of the patient. A second hollowed out portion being configured to house a heating arrangement, which heating arrangement comprises a chemical heat pack and/or a battery-operated electric heating arrangement and a cover for the heating arrangement being disposed to form a closing side of said second hollowed out portion and being configured to provide an enclosure for the heating arrangement disposed in said second hollowed out portion. The cover comprising at least one of: a heat chamber cover; a microfiber holder; and a microfiber covering layer. The cover further comprises a hole therein being configured to receive a tip of a laparoscope. The procedure comprising: wetting said sponge by impregnating said sponge with cleaning solution, and disposing said impregnated sponge in said first hollowed out portion of said cleaning device; inserting said heating arrangement in said second hollowed out portion of said cleaning device; snapping said cleaning device onto the trocar and orienting said cleaning device toward a surgeon such that the apex of said isosceles triangle is disposed towards the surgeon or in some other direction than the apex being disposed towards said surgeon; heating the heating arrangement to a predetermined temperature; initially inserting a laparoscope into the trocar and into the body of a patient; proceeding with surgery until the lens portion of the laparoscope at its tip becomes smeared with the patient's bodily components or until the lens of the laparoscope is unusable because of condensation on the lens; removing the laparoscope from the trocar and wiping the lens on said impregnated sponge until the bodily components or condensate has been removed; inserting the tip of the laparoscope with its lens through said hole in said heat chamber cover; raising the temperature of the laparoscope tip sufficiently to minimize condensation on the laparoscope lens during a next phase of the surgical procedure; removing the tip of the laparoscope from said heating chamber and inserting the tip of the laparoscope into the trocar; continuing the surgical procedure with a cleaned laparoscope; and repeating the above cleaning operation when required.

Another feature or aspect of an embodiment is a cleaning device for cleaning laparoscopes during a medical procedure for performing laparoscopic surgery, said cleaning device comprising a body with a snap device attached to said body, which snap device is configured to attach to a trocar. The body comprises an isosceles triangular shape with a longer side connecting the equal sides of the isosceles triangle with the snap device being attached to the middle of said longer side of the isosceles triangle and the body comprising two hollowed out portions, one of said hollowed out portions being disposed on each side of said snap arrangement. A first hollowed out portion being configured to house a sponge impregnated with a cleaning solution, which cleaning solution is configured to clean off the tip portion of a laparoscope, where a lens is disposed, which tip portion is configured to be inserted into the trocar and into the body of the patient. A second hollowed out portion being configured to house a heating arrangement, which heating arrangement comprises a chemical heat pack and/or a battery-operated electric heating arrangement and a cover for the heating arrangement being disposed to form a closing side of said second hollowed out portion and being configured to provide an enclosure for the heating arrangement disposed in said second hollowed out portion. The cover comprising at least one of: a heat chamber cover; a microfiber holder; and a microfiber covering layer. The cover further comprises a hole therein being configured to receive a tip of a laparoscope. According to another embodiment, a cleaning device configured for cleaning a surgical tool prior to insertion of the tool into a body of a patient during minimally invasive surgery includes a housing having at least one opening for accessing an interior of the housing; a sponge and a heater assembly positioned in the interior of the housing; and a connector. The connector includes a first arm and a second arm extending from an outer surface of the housing. An inner surface of the first arm and an inner surface of the second arm define at least a first recess sized to receive a sidewall of a first tubular body having a first diameter, and a second recess sized to receive a sidewall of a second tubular body having a second diameter, different than the first diameter. The connector is configured to removably attach the housing to the sidewall of the first tubular body or to the sidewall of the second tubular body, thereby supporting the housing relative to the first tubular body or the second tubular body.

According to another embodiment, a trocar assembly for introducing a surgical scope to a body during a minimally invasive surgery includes: a trocar having a tubular body with a sidewall having a first maximum outer diameter; and a cleaning device configured to removably mount to the sidewall of the tubular body of the trocar. The cleaning device includes: a housing having at least one opening for accessing an interior of the housing; a sponge and a heater assembly positioned in the interior of the housing; and a connector. The connector includes a first arm and a second arm extending from an outer surface of the housing. An inner surface of the first arm and an inner surface of the second arm define at least a first recess sized to receive the sidewall of the tubular body of the trocar and a second recess sized to receive a sidewall of a tubular body of a trocar having a second diameter which is smaller than the first diameter. The connector is configured to removably attach the housing to the trocar to support the housing relative to the trocar.

Examples of the present application will now be described in the following numbered clauses:

Clause 1: A cleaning device configured for cleaning a surgical tool prior to insertion of the tool into a body of a patient during minimally invasive surgery, the cleaning device comprising: a housing comprising at least one opening for accessing an interior of the housing; a sponge and a heater assembly positioned in the interior of the housing; and a connector comprising a first arm and a second arm extending from an outer surface of the housing, wherein an inner surface of the first arm and an inner surface of the second arm define at least a first recess sized to receive a sidewall of a first tubular body having a first diameter, and a second recess sized to receive a sidewall of a second tubular body having a second diameter, different than the first diameter, and wherein the connector is configured to removably attach the housing to the sidewall of the first tubular body or to the sidewall of the second tubular body, thereby supporting the housing relative to the first tubular body or the second tubular body.

Clause 2: The cleaning device of clause 1, wherein the tubular body comprises a tubular portion of a surgical trocar.

Clause 3: The cleaning device of clause 1 or clause 2, wherein the connector supports the housing, such that a central longitudinal axis of a portion of the first tubular body received within the first recess or of the second tubular body received within the second recess of the connector is spaced apart from the interior of the housing.

Clause 4: The cleaning device of any of clauses 1 to 3, wherein the connector supports the housing, such that a line normal to a bottom surface and passing through the opening of the housing is parallel to and a fixed distance from a central longitudinal axis of the portion of the first tubular body received within the first recess or of the second tubular body received within the second recess of the connector.

Clause 5: The cleaning device of any of clauses 1 to 4, wherein the at least one opening is sized such that a lens of the surgical tool can be inserted through the at least one opening to access the sponge and/or the heater assembly.

Clause 6: The cleaning device of any of clauses 1 to 5, wherein the first arm and the second arm of the connector are configured to deflect radially outwardly from the first recess and/or the second recess to receive the first or second tubular body and to move radially inwardly to engage a portion of a sidewall of the first or second tubular body upon insertion of the tubular body into the first recess and/or the second recess.

Clause 7: The cleaning device of any of clauses 1 to 6, wherein the first arm and the second arm comprise a first end mounted to a portion of an outer surface of the housing and a free second end opposite the first end, the free second end comprising a protrusion comprising an inwardly angled outer surface configured to direct the sidewall of the first or second tubular body into the first recess and/or the second recess.

Clause 8: The cleaning device of clause 7, wherein an inwardly facing surface of the protrusion is configured to engage the sidewall of the first tubular body of the first diameter to maintain the first tubular body within the first recess.

Clause 9: The cleaning device of clause 7 or clause 8, wherein the first recess is accessible through a space between the protrusion of the first arm and the protrusion of the second arm, and wherein the second recess is accessible from the first recess through a space between portions of the first arm and the second arm other than the protrusions.

Clause 10: The cleaning device of any of clauses 1 to 9, wherein the first diameter is from about 6.0 mm to about 18.0 mm and the second diameter is from about 1.0 mm to about 6.0 mm.

Clause 11: The cleaning device of any of clauses 1 to 10, wherein an inner surface of the first arm and an inner surface of the second arm each comprise a first curved portion having a first radius sized such that the first curved portion engages a sidewall of the first tubular body, and a second curved portion having a radius sized such that the second curved portion engages a sidewall of the second tubular body.

Clause 12: The cleaning device of any of clauses 1 to 11, wherein the first arm and/or the second arm further define at least one third recess sized to receive and engage a sidewall of a third tubular body having a third diameter, which is less than the first diameter or the second diameter, and wherein the third recess is accessible through a space between the inner surface of the first arm and the inner surface of the second arm which forms a portion of the second recess.

Clause 13: The cleaning device of any of clauses 1 to 12, wherein portions of the first arm and/or the second arm configured to contact a sidewall of the first tubular body and/or the second tubular body comprise textured regions configured to enhance a frictional engagement between the sidewall of the tubular body and the inner surface of the first arm and/or the second arm.

Clause 14: The cleaning device of clause 13, wherein the textured regions comprise a plurality of longitudinally extending ribs extending radially inwardly from inner surfaces of the first arm and/or the second arm.

Clause 15: The cleaning device of any of clauses 1 to 14, further comprising a cloth wiping element adhered to a portion of an outer surface of the housing for wiping fluid from a lens of the surgical tool.

Clause 16: The cleaning device of any of clauses 1 to 15, wherein the housing comprises: a base integrally formed with the connector; a cover comprising an open bottom connected to the base, a partially closed top, and an annular sidewall extending therebetween, wherein the at least one opening of the housing extends through the top of the cover; and a tubular fluid reservoir comprising an open top accessible through the at least one opening of the cover and a closed bottom mounted to the base.

Clause 17: The cleaning device of clause 16, wherein the at least one sponge is held in an interior of the fluid reservoir by a frictional engagement between an inner sidewall of the fluid reservoir and an outer annular surface of the at least one sponge.

Clause 18: The cleaning device of clause 16, wherein the heater assembly comprises a conductive film wrapped around at least a portion of a sidewall of the fluid reservoir; an insulating film wrapped around at least a portion of the conductive film of the conductive film; and a thermostat electrically connected between the conductive film and a power source, configured to disconnect the conductive film from the power source when the thermostat measures a temperature above a target value.

Clause 19: The cleaning device of any of clauses 16 to 18, wherein the base further comprises at least one battery terminal, configured to receive at least one battery for powering the heater assembly, and wherein the battery terminal holds the battery in a position, in which a longitudinal axis of the battery is parallel to a longitudinal axis of a portion of the tubular body received by the connector.

Clause 20: A trocar assembly for introducing a surgical scope to a body during a minimally invasive surgery, the assembly comprising: a trocar comprising a tubular body comprising a sidewall having a first maximum outer diameter; and a cleaning device configured to removably mount to the sidewall of the tubular body of the trocar, the cleaning device comprising: a housing comprising at least one opening for accessing an interior of the housing; a sponge and a heater assembly positioned in the interior of the housing; and a connector comprising a first arm and a second arm extending from an outer surface of the housing, wherein an inner surface of the first arm and an inner surface of the second arm define at least a first recess sized to receive the sidewall of the tubular body of the trocar, and a second recess sized to receive a sidewall of a tubular body of a trocar having a second diameter which is smaller than the first diameter, and wherein the connector is configured to removably attach the housing to the trocar to support the housing relative to the trocar.

For purposes of the description hereinafter, the spatial orientation terms and derivatives thereof shall relate to the exemplification as it is oriented in the drawing figures. However, it is to be understood that the present application may assume various alternative variations, except where expressly specified to the contrary. It is also to be understood that the specific devices illustrated in the attached drawings, and described in the following specification, are simply exemplary exemplifications. Hence, specific dimensions and other physical characteristics related to the exemplifications disclosed herein are not to be considered as limiting.

These and other features and characteristics of the present application, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. As used in the specification and the claims, the singular form of “a”, “an”, and “the” include plural referents unless the context clearly dictates otherwise.

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation an object, the term “proximal” refers to portions of the device nearest to a center or center of mass of the object. The term “distal” refers to a portion of the object farthest away from the center or center of mass of the device. For example, for a scope cleaning device, portions of the device located in the interior of the device housing are “proximal” relative to portions of the device connected to and extending from an outer surface of the device housing. When used in connection with a tool, such as a surgical or medical device, such as a surgical scope or trocar, the term “proximal” refers to the portion of the device configured to be handled by a user. The term “distal” refers to portions of the device opposite the proximal side of the device (e.g., portions of the device farthest away from the portions of the device handled by the user). It is also to be understood, however, that the specific devices and processes illustrated in the attached drawings, and described in the following specification, are simply exemplary exemplifications of the disclosure. Hence, specific dimensions and other physical characteristics related to the exemplifications disclosed herein are not to be considered as limiting.

FIG. 12 shows a trocar 310 used in minimally invasive surgery. The trocar 310 has a cleaning device 312 removably connected thereto. The cleaning device 312, as shown, is snapped onto the trocar 310 using a snap member 314. However, this is not to be construed as limiting the present disclosure as any suitable attachment element, such as a hook-and-loop fabric, buckles, clips, tape, etc., configured to securely and removably attach the cleaning device 312 to the trocar 310 may be utilized.

An exemplification of cleaning device 312 is depicted in FIG. 12 in an exploded view. The cleaning device 312 comprises a casing 315 having the snap member 314 attached to an outside rear surface thereof. A first depression 324 may be formed in an upper surface of the casing 315 of cleaning device 312 to receive a sponge 320. In one example, the sponge 320 may be impregnated with a cleaning medium 322. The casing 315 may further include a second depression 326 formed in an upper surface thereof and disposed opposite the first depression 324. The second depression 326 may be configured to receive a heating element therein. In one example, the heating element can comprise batteries 316 and a heating coil 318 operatively connected to the batteries 316. The heating coil 318 may be configured to warm a microscope, laparoscope, or other surgical scope (not shown) so that the scope is ready for reinsertion in the trocar 310 and ready for reinsertion into the body of the patient being operated upon. Instead of batteries 316 and heating coil 318, a chemical pack housing chemicals that combine in an exothermic reaction, such as is used by skiers in their gloves to keep their hands warm while skiing, may be used as the heating element.

In one exemplary use of the cleaning device 312, the scope being utilized in a surgical procedure is removed from the trocar 310. It is first wiped off on the sponge 320, thereby to remove matter from the patient which has built up during the surgical procedure or which has condensed upon the scope during the surgical procedure. The heating element is then used to heat the front of the scope, thereby heating the front of the scope above the temperature of the patient to discourage the formation of condensate on the front of the scope when the scope is within the body of the patient. One or more lights 336, such as light emitting diode (LED), may be positioned on casing 315 and used to provide illumination for the trocar 310 and the site where the trocar 310 is inserted into a patient. The lights may be operatively connected to batteries 316.

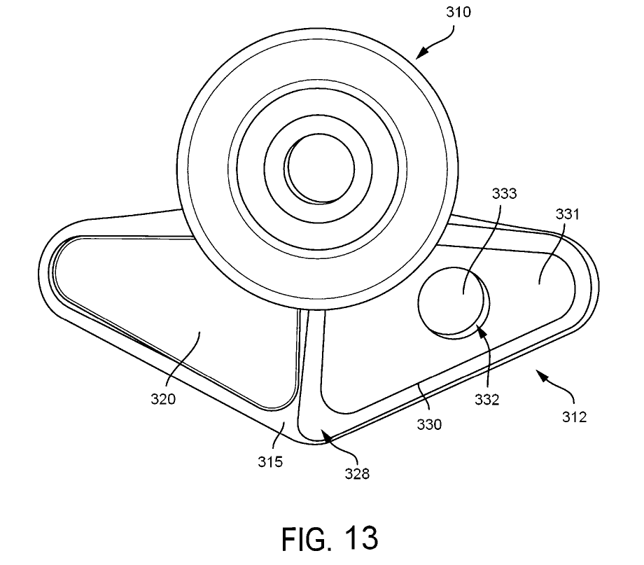

FIG. 13 shows a top view of cleaning device 312 attached to trocar 310. The second depression 326 may further include a heating chamber cover 328 positioned over the top thereof to provide protection for the heating element. The heating chamber cover 328 may further include a microfiber holder 330 formed therein configured to hold a microfiber cover 331. The heating chamber cover 328 and the microfiber cover 331 may be configured with at least one scope access hole 332 which permit the microscope or other scope to be inserted into heating chamber 333 to be warmed by heating coil 318, a heating pack, or some other manner known in the art. Microfiber cover 331 may be used by surgical staff to wipe debris and/or fluids from a scope in order to clean the optics thereof prior to inserting the scope into the scope access hole 332 to be heated. In an exemplification, microfiber cover 331 may be disposed on heating chamber cover 328 within the holder 330 in order to provide easy access to surgical staff.

With further reference to FIG. 13, in one example, casing 315 may have a substantially isosceles triangular shape when viewed from above, with the two sides of the triangle that are not of equal length not adjacent to the trocar 310. Such a configuration may have the benefit of optimizing placement of the heating coil 318 and batteries 316 in casing 315 next to sponge 320. This configuration also may have the benefit of providing easy access to cleaning device 312 while minimizing interference of the device with access to the surgical site while the cleaning device 312 is attached to the trocar 310 during a surgical procedure.

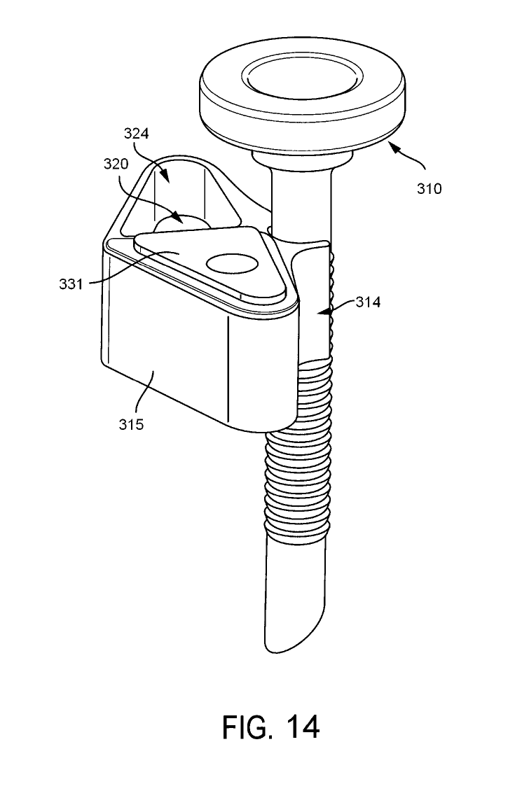

In FIG. 14, the trocar 310 is shown again with the cleaning device 312 attached thereto by snap member 314. A possible advantage of such an exemplification is that it places cleaning device 312 in close proximity to trocar 310, and thereby provides easy access to surgical staff. Such an exemplification also has the possible advantage that cleaning device 312 does not need to be set on a separate tray.

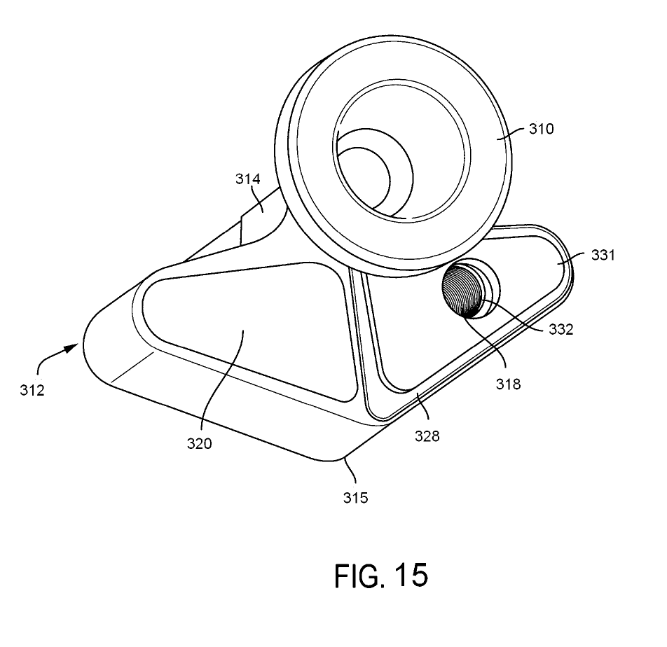

With reference to FIGS. 14 and 15, sponge 320 is shown in its installed position in first depression 324 in casing 315. Sponge 320 may be comprised of any material known in the art. Sponge 320 may be used for cleaning debris and/or fluid from a scope. Sponge 320 also may be impregnated with cleaning medium 322, such as a cleaning and/or defogging fluid. In an exemplification, cleaning medium 322 and/or sponge 320 may be sterile.

FIG. 15 shows the trocar 310 with an exemplification of cleaning device 312 from a different angle than the previous figures. Holes in microfiber cover 331 and heating chamber cover 328 create the scope access hole 332, which allows a scope to access heating chamber 333 in second depression 326, where heating coil 318 may warm and aid in defogging a scope. Debris may be wiped from a scope's optical components by surgical staff on microfiber cover 331 and/or sponge 320, which may be impregnated with cleaning medium 322.

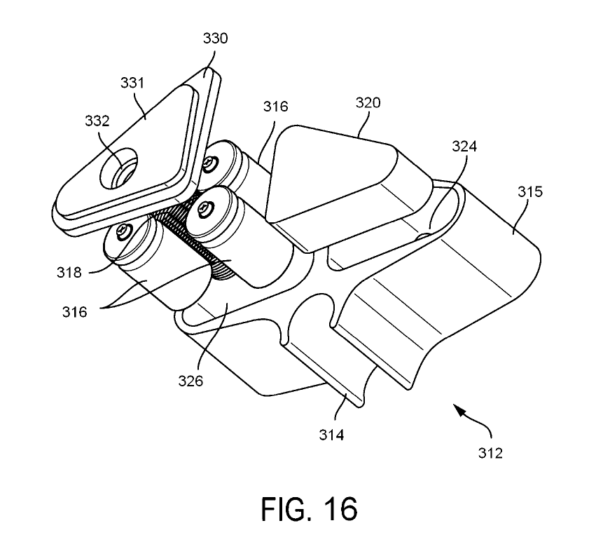

FIG. 16 is another exploded view of the cleaning device 312 having the casing 315. The casing 315 comprises an attaching mechanism, such as snap member 314, which is configured to attach to a trocar 310. Snap member 314 is configured to removably attach to trocar 310. Other exemplifications of casing 315 may be configured to attach to trocar 310 with tape, a friction fit, or through another mechanism otherwise known in the art.

The casing 315 also comprises a first depression 324, which is configured to hold a sponge 320. The sponge 320 may be at least partially moistened with cleaning medium 322. The casing 315 also comprises second depression 326 which is configured to hold batteries 316 and heating coil 318 or a defogger, and forms heating chamber 333. Second depression 326 may hold one or more batteries 316, which in an exemplification includes three lithium batteries. In other exemplifications, other types of batteries 316 may be used or adapted for use, and other numbers of batteries may be used. In another exemplification, another heating source other than an electrical or battery-powered heating source could be utilized, such as a chemical heat source or chemical heat pack, which would avoid the use and ultimate disposal of batteries, which can present challenges due to the potential environmental impact of battery waste.

The second depression 326 and heating chamber 333 of the casing 315 may be at least partially covered by heating chamber cover 328. The heating chamber cover 328 may comprise the microfiber holder 330 and scope access hole 332. A scope or microscope may be inserted into the scope access hole 332 and through the heating coil 318 or defogger. The scope or microscope may be wiped on the microfiber cover 331 disposed in the holder 330 before or after insertion into the scope access hole 332.

In at least one possible exemplification, a structure or material, such as double-sided adhesive tape, could be connected or attached to the casing 315, such as the side or underside, to allow the casing 315 to be connected or attached to a support structure.

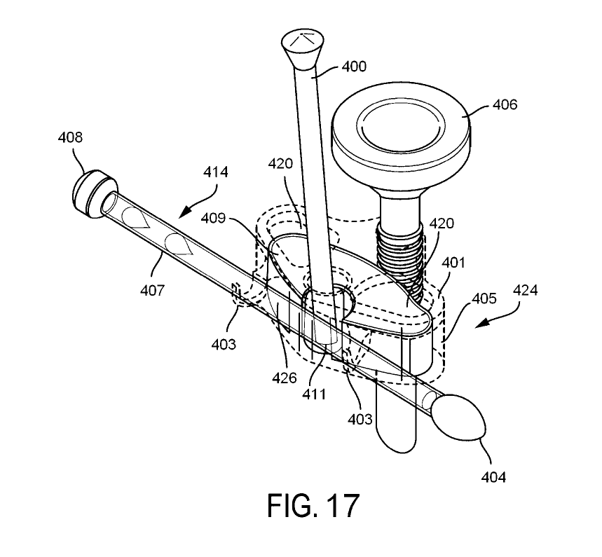

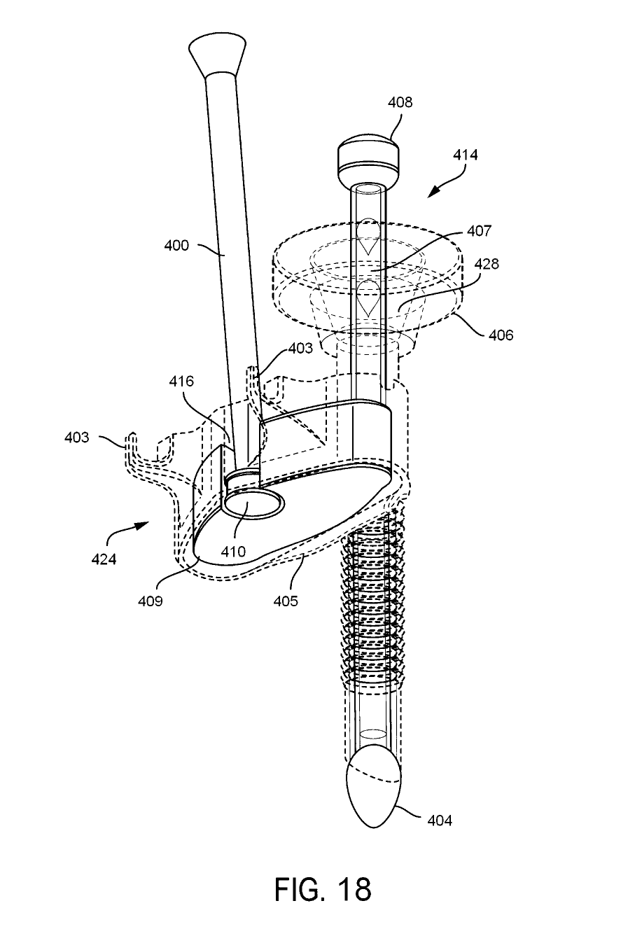

FIG. 17 shows a partially transparent view of an exemplification of a cleaning kit 424 attached to a trocar 406. The kit 424 includes a container 105 that may house a sponge 410, which may be impregnated with a cleaning solution, and a heater 409. A cover 401 is located on top of the container 405. The cover 401 has an opening therein into which can be inserted a surgical viewing device 400, such as a scope, laparoscope, or microscope, such that the scope lens 411 is inside the container 405. In an exemplification, cover 401 may include one or more recesses 420, at least one of which contains a microfiber wipe or cleaner 402. Also shown is a cannula cleaner 414, which is held by two cannula cleaner tube holders 403. Holders 403 may removably secure the cannula cleaner 414 to the container 405 when it is not in use during a surgical procedure. Cannula cleaner 414 may be secured by a friction fit, or in some other manner otherwise known in the art. In addition, the cover 401 may have one or more projections 426 that project out above the cannula cleaner 414 to further secure the cannula cleaner 414 in place in a secured position on container 405.

An exemplification of cannula cleaner 414 may include an elongated body with a cleaning tip 404 disposed at one end. In an exemplification, the body of the cannula cleaner 414 may comprise a tube that also functions as a solution container 407. On the end opposite the cleaning tip 404 is located a solution dispensing valve 408. Either the valve 408 or the solution container 407 may be squeezed, pinched, or pressed in to cause an amount of a cleaning or defogging solution, or some other liquid known in the art, to be dispensed. The solution may be dispensed into the container 405. By containing the solution in the cannula cleaner 414, a supply of cleaning solution is readily available to be dispensed into the container 405, and is in easy reach for a user of the trocar 406 and cleaning kit 424, such as by a surgeon or other medical professional. By mounting the cannula cleaner 414 on the container 405, the cannula cleaner 414 is easy to reach for use in cleaning or clearing out the trocar 406. The design of the cannula cleaner 414, therefore, combines a cleaning device with a cleaning solution supply, so as to facilitate keeping the trocar 406 and viewing instruments clean during a surgical procedure. However, it should be understood that other exemplifications of cannula cleaner 414 may exist, such as an exemplification that does not hold solution and lacks a solution dispensing valve 408. Another exemplification may include cleaning tips 404 at both ends of the cleaning device 414. In such an exemplification, the cleaning tips 404 may be of the same size or of different sizes. Cannula cleaning tip 404 may be comprised of a sponge, foam, microfiber, or some material otherwise known in the art.

FIG. 18 shows another view of the cleaning kit 424 shown in FIG. 17. In FIG. 18, container 405 is shown as transparent in order to demonstrate how scope 400 may be inserted through scope access hole 416, so that the optics thereof may contact sponge 410. However, it should be understood that not all exemplifications of container 405 need be transparent. FIG. 18 also depicts how scope 400 may interact with an exemplification of heater 409. In an exemplification, heater 409 may be configured to surround or partially surround scope 400, and may substantially border the perimeter or part of the perimeter of sponge 410. Heater 409 and sponge 410 may be configured to create a substantially cylindrical space, where the lens 411 of a scope 400 may be inserted for cleaning and/or defogging, which may be possible to engage exemplifications of scopes 400 that are substantially cylindrically-shaped. In an exemplification, sponge 410 may be configured to maximize efficient contact with lens 411 of scope 400. In an exemplification, sponge 410 may be substantially circular in shape. Exemplifications of heater 409 may comprise other shapes. Exemplifications of heater 409 may use electrical or chemical processes to generate heat, or may do so using another manner known in the art.

With further reference to FIG. 18, cannula cleaner 414 may be inserted into a cannula 428 of trocar 406 to clean the cannula 428 of fluids, condensates, and/or debris. In an exemplification, cannula cleaning tip 404 may be inserted into cannula 428. In an exemplification, cannula cleaning tip 404 may have a diameter that is substantially the same or larger than the diameter of the cannula 428. Cannula cleaning tip 404 may be comprised of a deformable material, and deform to fit into cannula 428 while contacting the walls of the cannula 428. Cannula cleaning tip 404 may be sufficiently absorptive to absorb fluids and/or condensate present in trocar 406. Trocar 406 is shown as transparent in order to better illustrate the insertion of cannula cleaner 414 therein; however, it should be understood that exemplifications of trocar 406 need not be transparent.

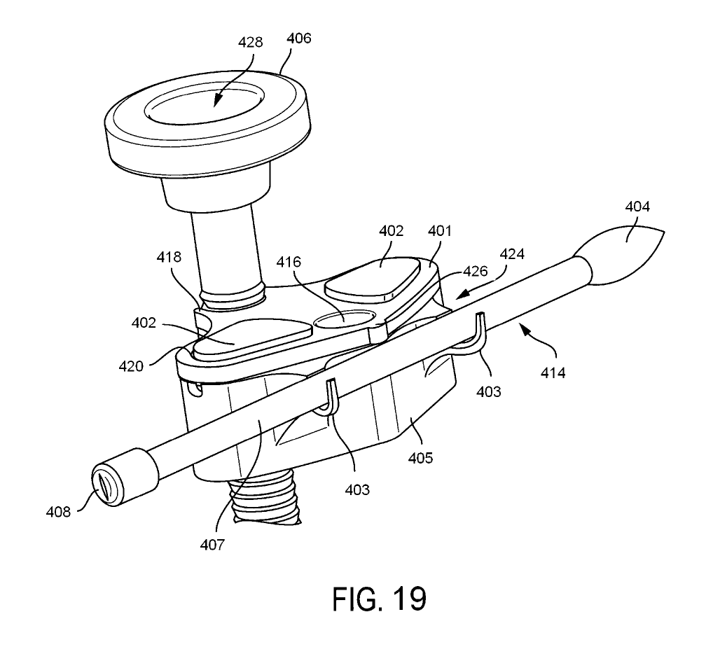

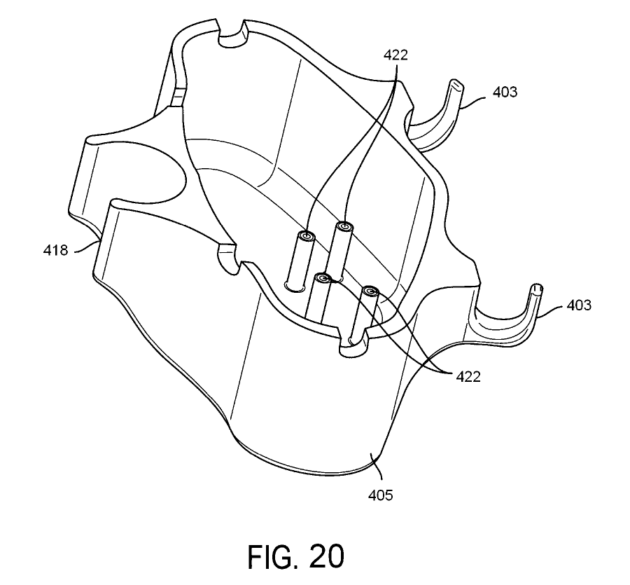

FIG. 19 shows a perspective view of the cleaning kit 424 depicted in FIG. 17. Cannula cleaner 414 is shown disposed on cannula holders 403, which are configured to contain cannula cleaning device 414 against container 405 during a surgical procedure when cannula cleaner 414 is not in use. In an exemplification, cover 401 of cleaning kit 424 may include projection 426 that is configured to further constrain cannula cleaner 414 when not in use. Cover 401 may include one or more microfiber wipes 402 disposed thereon to further aid in cleaning debris or condensate from a scope 400. While the exemplification shown in FIG. 19 shows a configuration wherein two microfiber wipes 402 are positioned on cover 401 roughly on either side of scope access hole 416, it should be understood that other configurations or numbers of wipes 402 may be used. It also should be understood that exemplifications of cover 401 need not include microfiber wipes 402, and that other materials or wiping elements otherwise known in the art may be used to wipe debris from the lens 411 of a scope 400. Wipes 402 may be disposed in one or more recesses 420 in cover 401. Cleaning kit 424 may removably attach to trocar 406 by a snap member 418, or another manner known in the art.

FIG. 20 shows a perspective view of a portion of the container 405 of the cleaning kit 424 shown in FIG. 17. An exemplification of container 405 may be integrally molded with snap member 418 and/or one or more cannula cleaning device holder 403. The interior of container 405 may comprise a single chamber, or multiple chambers. The at least one chamber may include one or more support posts 422. Support posts 422 may provide support for cover 401, and may provide support for configurations of heater 409. In exemplifications of heater 409, posts 422 may support configurations of batteries, a heating coil, chemical packs, or other components of a heater 409 otherwise known in the art.

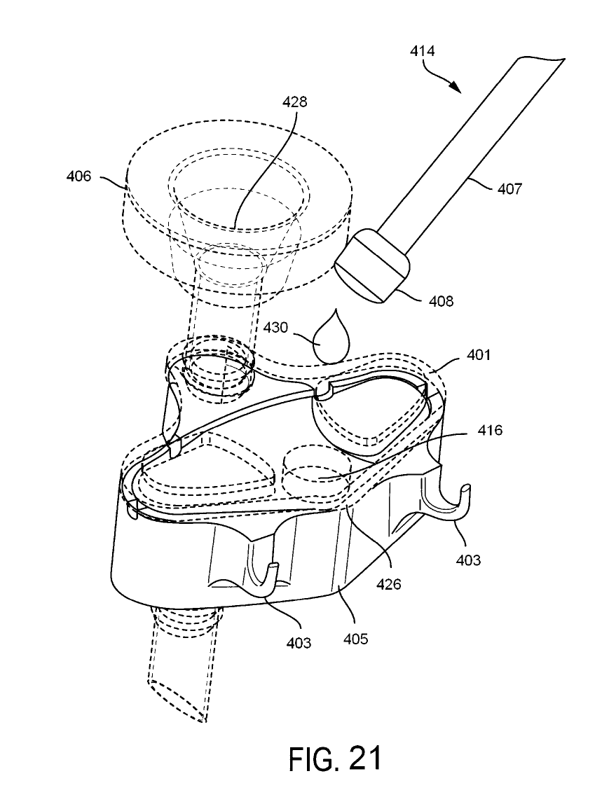

FIG. 21 shows another view of the cleaning kit 424. In an exemplification, cannula cleaner 414 may be used to dispense cleaning and/or defogging solution into container 405 through scope access hole 416. In an exemplification, solution may exit solution container 407 of cannula cleaner 414 through solution dispensing valve 408. When solution—represented by a droplet of cleaning solution 430—enters through scope access hole 416, it may contact and be absorbed by sponge 410. A scope may then be inserted into container 405 through scope access hole 416 and contact impregnated sponge 410, which may aid in cleaning and/or defogging the scope 400.

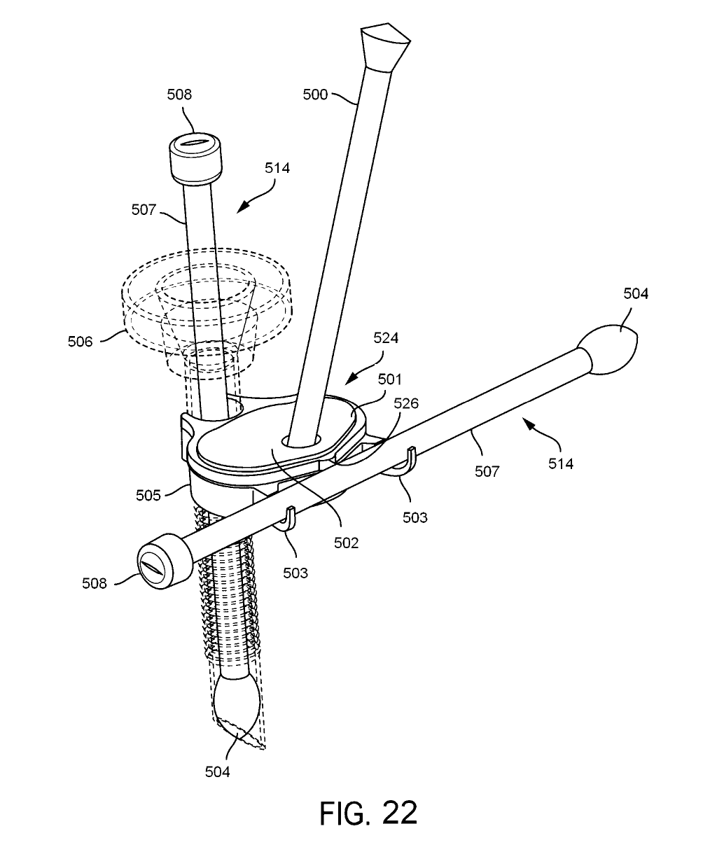

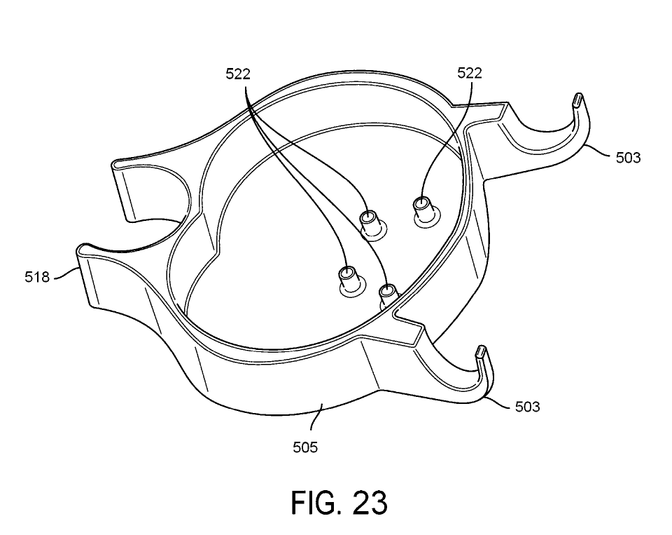

With reference to FIGS. 22 and 23, an alternative exemplification of a cleaning kit 524 is illustrated. Cleaning kit 524 is configured to clean lens 511 of a scope 500 and may include a container 505 that is shorter or thinner than the exemplification of the container 405 shown in FIG. 17. In the example in FIG. 22, no batteries are utilized, so a shorter or thinner container 505 can be utilized. In an exemplification, the kit may use no heating device or heating medium. In another exemplification, a chemical heating medium or chemical heating pack may be used which takes up less space than batteries. Cover 501 also may include a microfiber wipe or cleaner 502 that spans most or all of the surface area of cover 501. FIG. 22 depicts two cannula cleaners 514 in order to demonstrate both insertion into trocar 506, and placement in cleaner tube holders 503. However, it should be understood that not all exemplifications of cleaning kit 524 require more than one cannula cleaner 514. Each of the cannula cleaners may include a cannula cleaning tip 504 at a first end thereof, a solution container 507 extending between the first end and second end thereof, and a solution dispensing valve 508 positioned at the second end.

FIG. 23 shows a view of container 505 of the exemplification of the cleaning kit 524 shown in FIG. 22. In an exemplification, snap member 518 and one or more cannula cleaner holders 503 may be integrally molded with container 505. The container 505 may comprise a chamber, and may include one or more internal supports 522. In one example, internal supports 522 may be used to support cover 501, or constrain a heater and/or sponge.

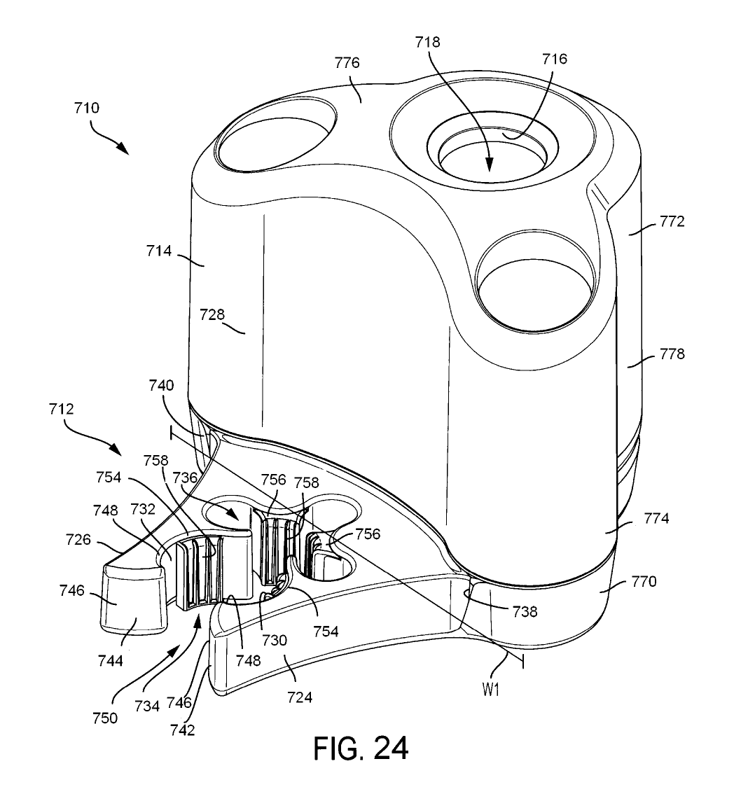

With reference to FIGS. 24-30, another exemplification of cleaning device 710 configured for cleaning a surgical tool, such as a surgical scope, prior to insertion of the tool into a body of a patient during minimally invasive surgery, is illustrated. As described herein, the cleaning device 710 includes a connector 712, such as a snap fit connector, which is configured to removably mount the cleaning device 710 to tubular or cylindrical objects having different maximum outer diameters without needing to adjust or reposition the connector 712 to accept and engage the different sized structures. As used herein, the cleaning device 710 is described as being connected to trocars used in surgical procedures. However, it is understood that the connector 712 can also be used for mounting the cleaning device 710 to other tubular bodies and structures including, for example, rods, posts, railings, pieces of furniture, as well as other medical devices and tools located at a surgical scene.

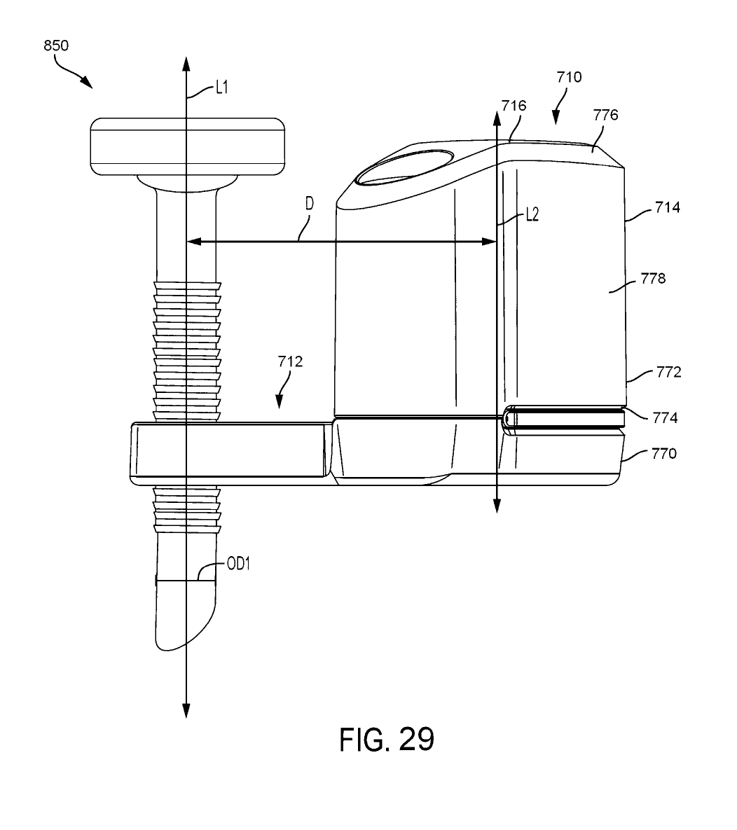

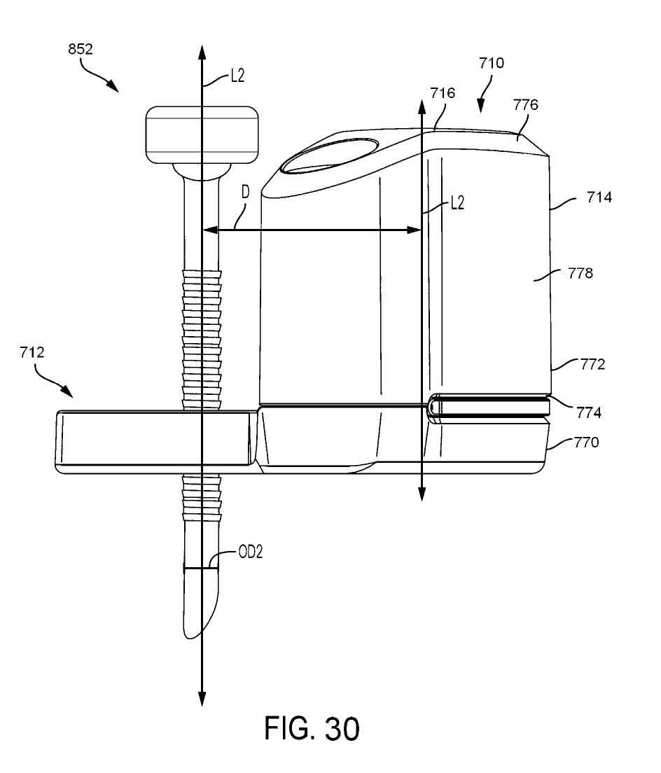

In some examples, the connector 712 can be configured to removably attach the cleaning device 710 to two different standard sized surgical trocars 850, 852 (shown in FIGS. 29 and 30). For example, the connector 712 can be configured to receive a large diameter trocar 850 (shown in FIG. 29), such as a trocar having a maximum outer diameter OD1 of from 6.0 mm to 18.0 mm, in one possible exemplification having a maximum outer diameter of about 8.0 mm. The connector 712 can also be configured to receive a smaller diameter trocar 852 (shown in FIG. 30), such as trocar having an outer maximum diameter OD2 of from 1.0 mm to 6.0 mm, in one possible exemplification about 4.0 mm. The connector 712 is configured to connect to one trocar at a time (either the single small (e.g., 4.0 mm) diameter trocar or the single large diameter (e.g., 8.0 mm) trocar). While the connector 712 may connect to other similar sized trocars (e.g., trocars having a maximum outer diameter within 5% or 10% of the designated size), the connector 712 is not designed to connect to a wide range of different trocar sizes. For example, the connector 712 is not designed to be adjusted or reconfigured for use with a range of trocar diameters. Desirably, portions of the connector 712 are naturally biased or sized to frictionally engage the trocar to hold the connector 712 and cleaning device 710 in place relative to the trocars 850, 852. Therefore, as described herein, the user does not need to, for example, tighten the connector 712 to secure the connector 712 to the trocar, cinch down a portion of the connector against the trocar, or perform some other action for adjusting the connector 712 to receive different sized trocars. Instead, the user need only insert the tubular portion of the trocar of either size (e.g., either 4.0 mm trocar or the 8.0 mm trocar) into the connector 712 to removably attach the cleaning device 712 to the trocar.

In some examples, the cleaning device 710 includes a protective outer structure such as a housing 714, casing, or other enclosure having at least one opening 716 for accessing an interior 718 of the housing 714. The housing 714 can be formed from any suitable rigid and substantially fluid tight material, such as plastic, rubber, ceramics, glass, or metal. Desirably, the housing 714 is lightweight and can be supported by a surgical tool, such as the trocar, without damaging the housing 714 or cleaning device 710. Accordingly, such housings are often formed from rigid lightweight plastics (e.g., polyesters, copolyesters, polyethylene terephthalate (PET), polystyrene, high-density polyethylene, polycarbonate, or similar materials).

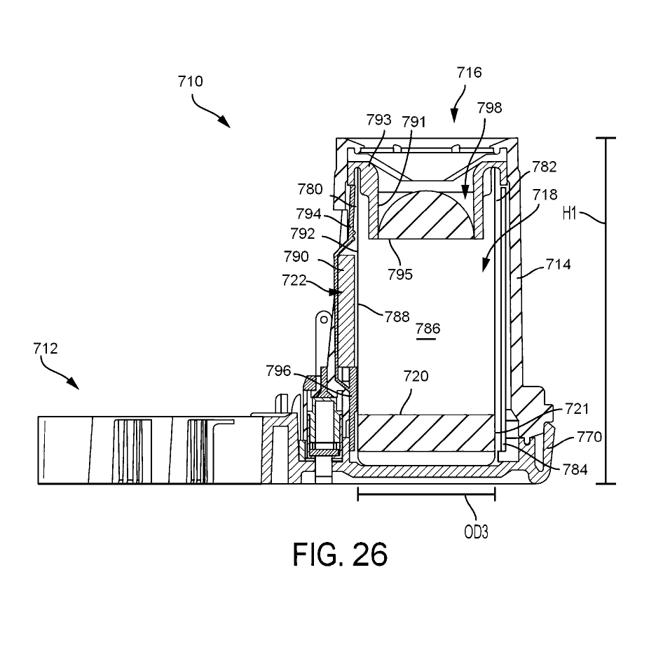

As in previous exemplifications, the cleaning device 710 also includes a sponge 720 (shown in FIG. 26) immersed in a defogging solution and a heater assembly 722 (shown in FIG. 26) in an interior 718 of the housing 714. For example, the sponge 720 can be a circular or cylindrical sponge having an outer diameter OD3 (shown in FIG. 26) of from about 15 mm to about 25 mm. The housing 714 and housing interior 718 are generally sized to receive a lens of a surgical tool, such as a laparoscope, endoscope, or cytoscope. Accordingly, dimensions of the housing 714 and interior 718 are selected so that the lens of the scope can be easily inserted into the interior 718 through the opening 716 to contact the sponge 720 and/or to be brought into proximity to the heater assembly 722. For example, the housing 714 can be from about 40 mm to 60 mm in height H1 and about 50 mm to about 70 mm in width W1.

The cleaning device 710 further includes the connector 712, which is configured to removably attach the housing 714 to the trocar, thereby supporting the housing 714 relative to the trocar. For example, the connector 712 can be configured to support the housing 714, such that a central longitudinal axis L1 (shown in FIGS. 29 and 30) of a portion of the trocar 850, 852 received by the connector 712 is spaced apart from the interior 718 of the housing 714. Similarly, the connector 712 can be configured to support the housing 714, such that a line L2 (shown in FIGS. 29 and 30) normal to a bottom surface and passing through the opening 716 of the housing 714 is parallel to and a fixed distance D1 (shown in FIGS. 29 and 30) from the central longitudinal axis L1 of the trocar 850, 852.

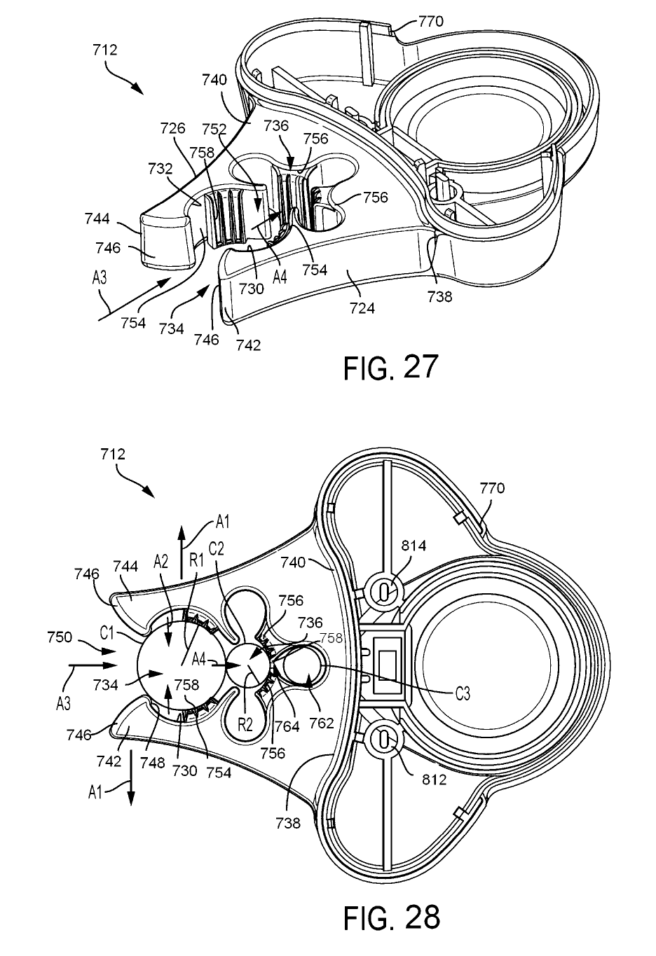

With specific reference to FIGS. 24-28, features of an exemplary connector 712 configured to receive the different sized trocars will now be discussed in detail. The connector 712 can include a first arm 724 and a second arm 726 extending from an outer surface 728 of the housing. The arms 724, 726 can be integrally formed with other portions of the housing 714. For example, the housing 714 and arms 724, 726 can be formed together by a suitable plastic molding process, such as injection molding. In other examples, the arms 724, 726 can be formed separately from other portions of the housing 714 and mounted to the outer surface 728 of the housing 714 by a suitable adhesive, fastener, or combination thereof. As described herein, the arms 724, 726 are sized to receive the different sized trocars and to hold the cleaning device 710 in a fixed position relative to the trocar. In order to receive the trocar, an inner surface 730 of the first arm 724 and an inner surface 732 of the second arm 726 may define at least a first recess 734, shown by circle C1 in FIGS. 27-28, sized to receive a trocar having a first diameter. The arms 724, 726 may also define at least one second recess 736, shown by circle C2 in FIGS. 27 and 28, sized to receive a trocar having a smaller diameter.

In order to permit mounting the cleaning device 710 to the trocar, the first arm 724 and the second arm 726 of the connector 712 can be configured to deflect radially outwardly, in a direction of arrow A1 (shown in FIGS. 27 and 28) from the first recess 734 and/or the second recess 336 to receive the trocar. The arms 724, 726 are also configured to move radially inwardly, in a direction of arrow A2 (shown in FIGS. 27 and 28) to engage the trocar.

In some examples, the first arm 724 and the second arm 726 include a first end 738, 740 mounted to a portion of the outer surface 728 of the housing 714 and a free second end opposite the first end 738, 740. In order to secure the trocar within the recess 734, 736, the arms 724, 726 can include a protrusion 742, 744 positioned at the free end of each arm 724, 726. The protrusions 742, 744 can include an inwardly angled outer surface 746 configured to direct the trocar into the first recess 734 and/or into the second recess 736. The protrusions 742, 744 can also include an inner surface 748 configured to engage the trocar to retain the trocar within the first recess 734. For example, the inner surface 748 can have a curvature which matches the curvature of the trocar.

The trocar enters the first recess 734 through a space 750 between the opposing protrusion 742 of the first arm 724 and the protrusion 744 of the second arm 726. For example, the user may press the trocar in a direction of arrow A3 through the space 750 and into the first recess 734. If the trocar is small enough (e.g., has a maximum outer diameter of less than about 6.0 mm) to pass from the first recess 734 into the second recess 736, the user can advance the trocar in a direction of arrow A4 into the second recess 736 through a second space 752 between portions of the first arm 724 and the second arm 726.

In some examples, the recesses 734, 736 are formed by curved portions or regions of the first arm 724 and the second arm 726. For example, the inner surface 730 of the first arm 724 and the inner surface 732 of the second arm 726 may each include a first curved portion 754 having a first radius R1 sized such that the first curved portion 754 engages a trocar of the first diameter OD1 (shown in FIG. 29). The arms 724, 726 can also include a second curved portion 756 having a radius R2, sized such that the second curved portion 756 engages a trocar with the second diameter OD2 (shown in FIG. 30).

In some examples, portions of the first arm 724 and/or the second arm 726 configured to contact the trocar can include textured or high friction surfaces 758 configured to enhance the frictional engagement between the trocar and the inner surface 730, 732 of the first arm 724 and/or the second arm 726. For example, the textured surface 758 can include a plurality of longitudinally extending ribs 760 extending radially inwardly from inner surfaces 730, 732 of the first arm 724 and/or the second arm 726.

In some examples, the connector 712 can also include a third recess 762 (shown by the circle C3 in FIGS. 27 and 28) for connecting the device 710 to a trocar of a third diameter, which is smaller than the first diameter or the second diameter. For example, the third recess 762 can be sized to receive a trocar with a maximum outer diameter of about 2.0 mm or less. The third recess 762 can be accessible through a space 764 between the portions of the arms 724, 726 that form the second recess 736.

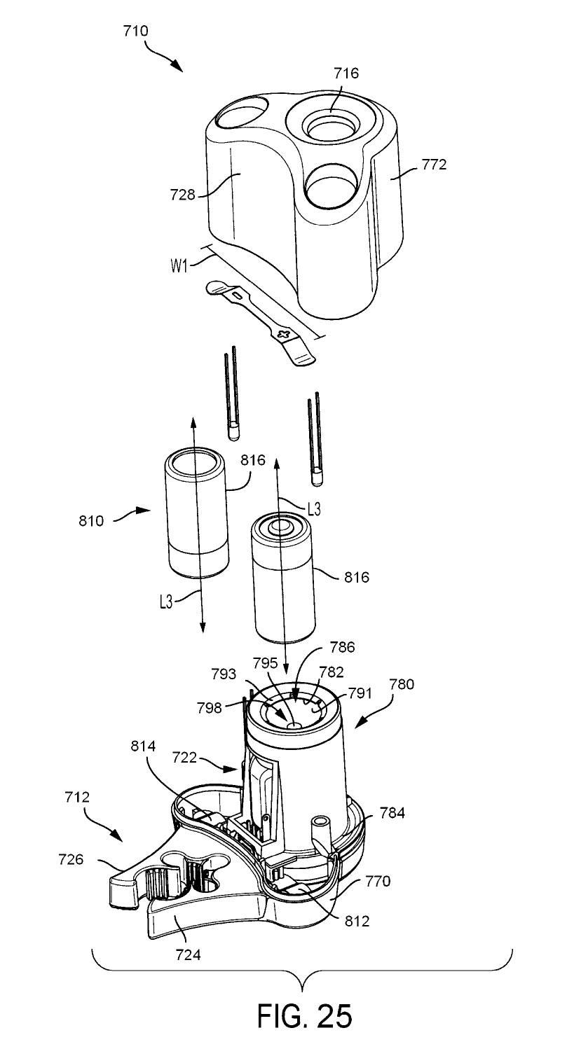

With reference again to FIGS. 24-26, in some examples, the housing 714 is formed from multiple individually formed or molded pieces joined together to form an enclosure. For example, the housing 714 can include a base 770 integrally formed with the connector 712. The housing 714 can also include a cover 772, such as a dome shaped cover, having an open bottom portion 774, a partially closed top portion 776, and an annular sidewall 778 extending therebetween. As shown in FIGS. 24 and 25, the at least one opening 716 for accessing the interior 718 of the housing 714 can be positioned on the top portion 776 of the cover 772. As described herein, additional structures formed from other materials can be attached to the base 770 and/or connector 712 using a suitable adhesive or molding process. For example, as described herein, the textured or high friction surface 758 of the connector 712 can be formed by overmolding a textured or high friction material to the connector 712 to enhance the frictional engagement between the connector 712 and trocar.

With reference to FIGS. 25 and 26, in some examples, the housing 714 further includes a tubular fluid reservoir 780 enclosed by the cover 772 and/or the base 770. The tubular reservoir 780 is sized to receive a portion of the scope for cleaning and defogging the scope. In some examples, the reservoir is about 35 mm to 45 mm tall and has an outer diameter of about 15 mm to 25 mm. The fluid reservoir 780 can include an open top 782 accessible through the at least one opening 716 of the cover 772 and a closed bottom 784 mounted to the base 770. The fluid reservoir 780 defines an interior 786, configured to receive cleaning solution to clean the scope. The at least one sponge 720 can be inserted into the interior 786 of the fluid reservoir 780. The sponge 720 can be a circular shape and sized such that a peripheral edge 721 of the sponge 720 engages an inner surface 788 of the fluid reservoir 780 by a frictional engagement to hold the sponge 720 in place in the fluid reservoir 780.

As in previous exemplifications, the heater assembly 722 is positioned in proximity to the fluid reservoir 780 and is configured to heat fluid and the sponge 720 contained therein. By heating the fluid and sponge to an appropriate temperature, the fluid effectively defogs a lens of the surgical scope, which improves a quality of images captured by the scope during a surgical procedure. Particularly, warming the lens of the scope to approximately body temperature reduces a likelihood that a lens of the scope will fog when it is inserted into the patient's body.

In some examples, the heater assembly 722 includes a conductive film 790 wrapped around at least a portion of an outer surface 792 of the fluid reservoir 780. The conductive film 790 can be any suitable conductive material, which increases in temperature when an electric current passes through the conductive film 790. Exemplary conductive materials include metallic films, such as films formed from copper, zinc, and similar materials. The conductive film 790 may also be a film formed from a conductive polymer material and/or a polymer film impregnated with conductive metallic particles. In other examples, the conductive film 790 can be replaced by other electrical circuitry for generating sufficient heat to warm the fluid reservoir 780 and surgical scope. For example, the heater assembly can include conductive wires, coils, foils, tape, or similar materials electrically connected to a power source for generating heat.

In some examples, the heater assembly 722 further includes an insulator 794 positioned around at least a portion of the conductive film 790 and the outer surface 792 of the fluid reservoir 780. For example, the insulator 794 can be an annular sleeve formed from an insulating material, such as silicone, neoprene, fiberglass, cotton, felt, or other insulating materials as are known in the art. In some examples, the insulator 794 can be molded or coated over the conductive film to provide protection for the film. In other examples, the insulator 794 is a separate sheet or sleeve wrapped around or positioned over the conductive film.

In some examples, the heater assembly 722 also includes a thermostat 796 electrically connected to a power source 810. The thermostat 796 can be configured to selectively apply power from the power source 810 to the conductive film 790, thereby causing the conductive film 790 to increase or decrease in temperature. The thermostat 796 can be mounted to other portions of the fluid reservoir 780 or at any other convenient location within the housing 714. In some examples, the thermostat 796 is configured to disconnect the power source 810 from the conductive film 790 when the thermostat 796 measures that the conductive film 790 and/or portions of the fluid reservoir 780 are above a target temperature value.

In some examples, the power source 810 includes battery terminals, such as a first battery terminal 812 and a second battery terminal 814, sized to receive one or more batteries 816. The batteries 816 can be conventional commercially available batteries, such as one or more of single A batteries, AA batteries, and/or a AAA batteries. For example, as shown in FIG. 25, the scope cleaner device 710 including two AA batteries 816. The battery terminals 812, 814 can be mounted to the base 770 of the housing 714 and configured to hold the batteries 816 in a position, in which a longitudinal axis L3 (shown in FIG. 25) of the battery 816 is parallel or is substantially parallel to a central longitudinal axis L1 (shown in FIG. 29) of a portion of the trocar received by the connector 712.

In some examples, the fluid reservoir 780 also includes an annular seal 798 connected to the open top 782 of the fluid reservoir 780. The annular seal 798 can be an elastomeric seal sized to receive the surgical device, such as the surgical scope, and to seal against a portion of the device to prevent fluid, such as defogging solution, from leaking from the interior 786 of the fluid reservoir 780. In some examples, the annular seal 798 includes a conical outer surface 791 extending radially inwardly from a peripheral edge 793 of the seal 798 to a narrow central opening 795. The annular seal 798 serves several purposes. First, it helps to maintain fluid in the fluid reservoir 780 by, for example, wiping excess fluid from the scope cleaner. The seal 798 also helps to prevent fluid from spilling out of the fluid reservoir 780 if the trocar, to which the cleaning device 710 is attached, is bumped, jostled, or moved. The seal 798 and central opening 795 can also be sized to facilitate filling the fluid reservoir 780 with defogging fluid. For example, a tip of a fluid bottle can be inserted into the interior 786 of the fluid reservoir 780 through the narrow opening 795. A top portion of the fluid bottle could rest against the conical surface 791 as the fluid reservoir 780 is being filled.

It is to be understood that the present application may assume various alternative variations, except where expressly specified to the contrary. It is also to be understood that the specific devices illustrated in the attached drawings, and described in the specification, are simply exemplary exemplifications of the present application. Although the present application has been described in detail for the purpose of illustration based on what is currently considered to be the most practical and possible exemplifications, it is to be understood that such detail is solely for that purpose and that the present application is not limited to the disclosed exemplifications, but, on the contrary, is intended to cover modifications and equivalent arrangements that are within the spirit and scope thereof. For example, it is to be understood that the present application contemplates that, to the extent possible, one or more features of any exemplification can be combined with one or more features of any other exemplification. The exemplifications of the present application described herein above in the context of the possible exemplifications are not to be taken as limiting the exemplifications of the present application to all of the provided details thereof, since modifications and variations thereof may be made without departing from the spirit and scope of the exemplifications of the present application.

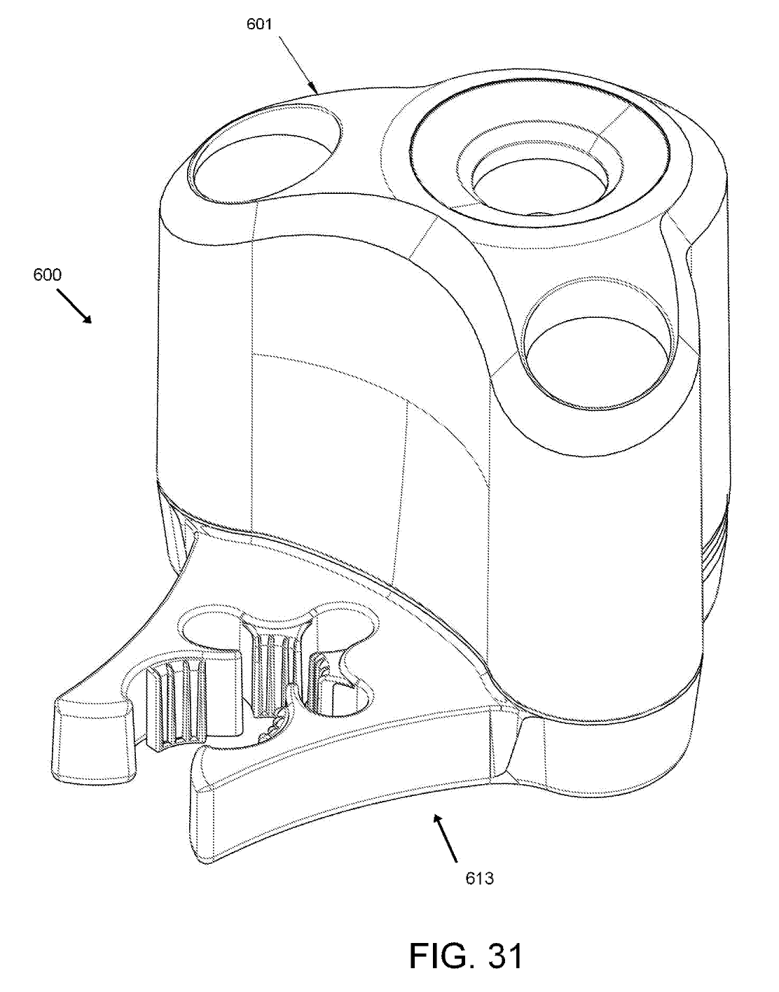

FIG. 31 is perspective view of a device 600, which comprises a top housing assembly 601 and an assembled bottom assembly 613.

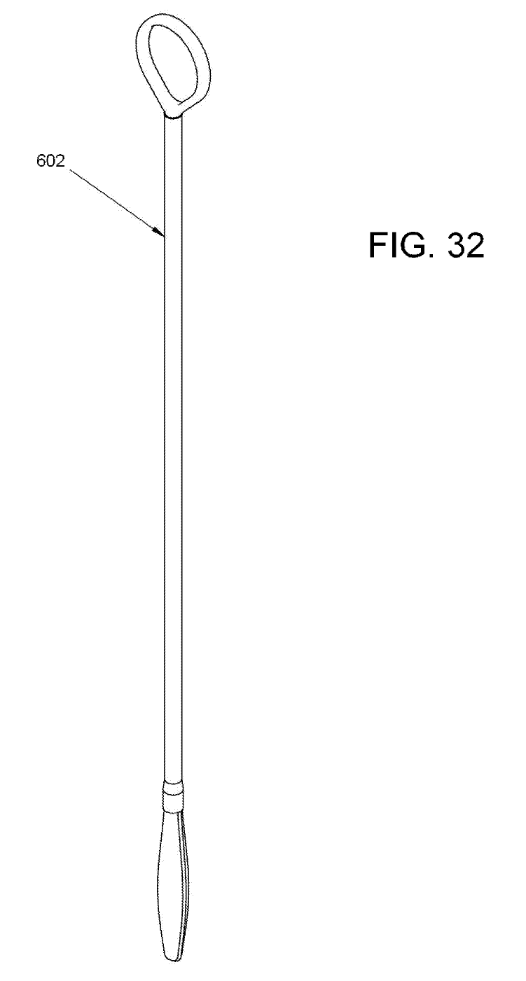

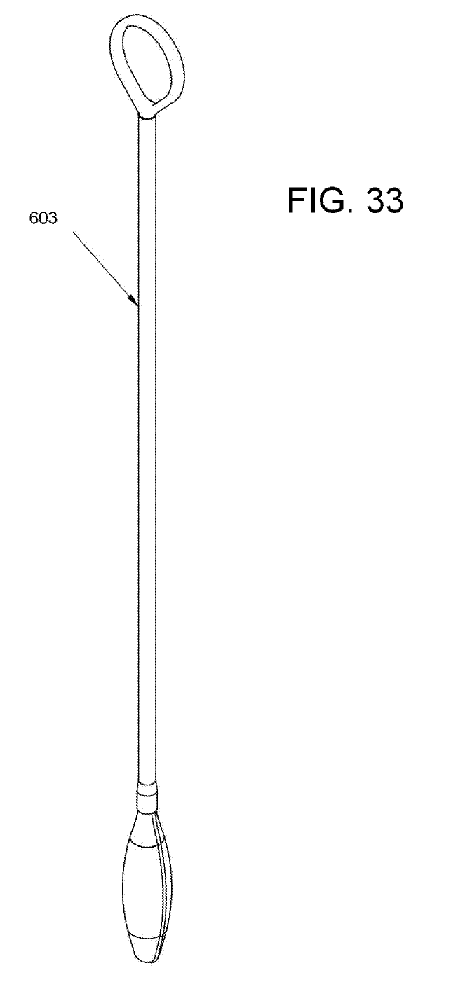

FIG. 32 shows a five millimeter wiping wand 602.

FIG. 33 shows a twelve millimeter wiping wand 603.

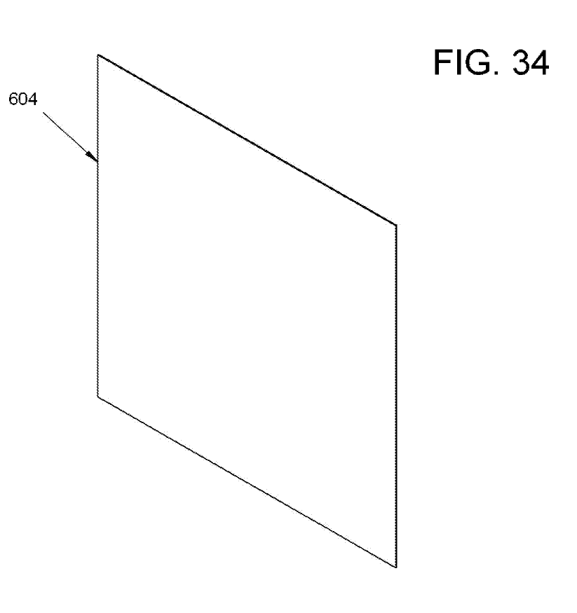

FIG. 34 shows a microfiber cloth 604.

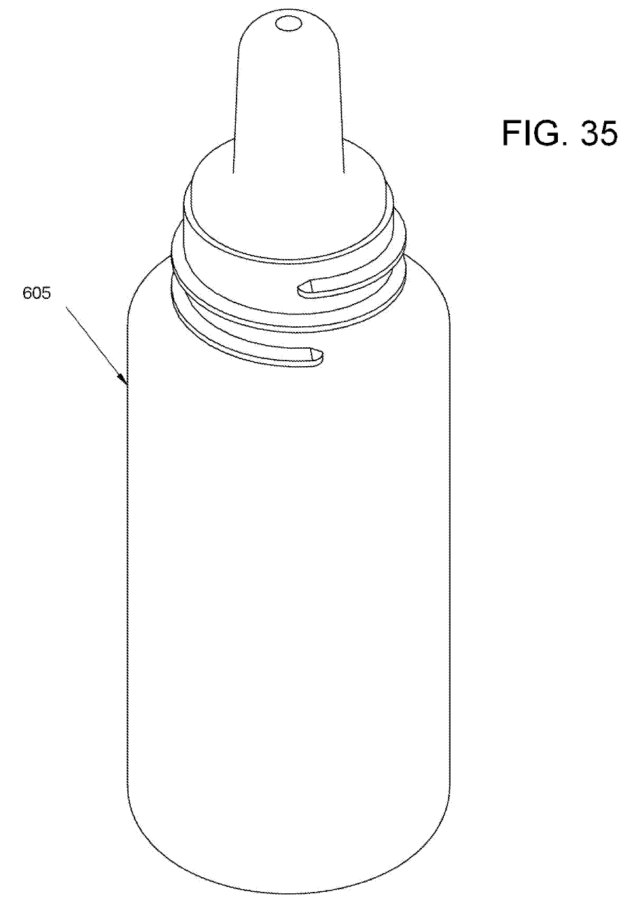

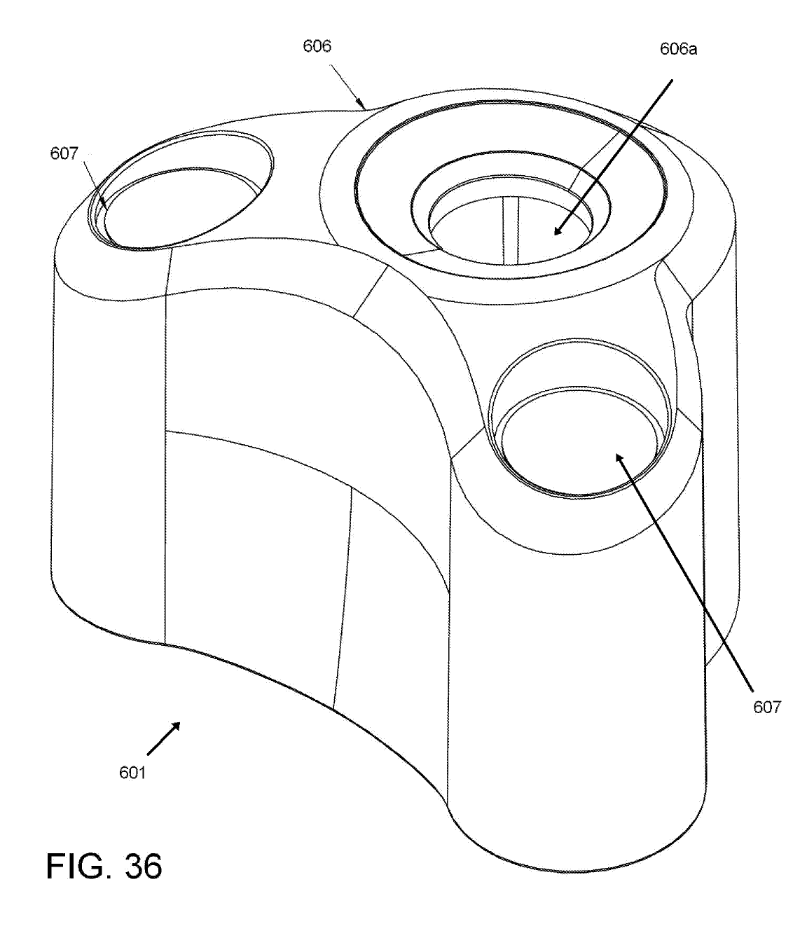

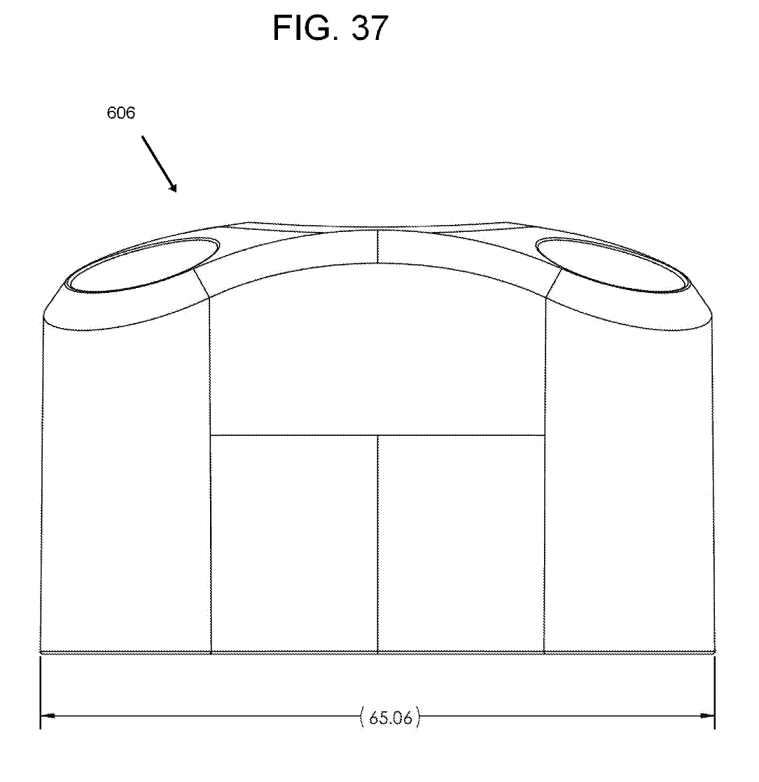

FIG. 35 shows a bottle of anti-fog solution 605.

FIG. 36 shows a housing top 606, a hole 606a, and two microfiber patches 607.

FIG. 37 shows a side view of the housing top 606. The housing top 606 has a possible width of 65.06 millimeters.

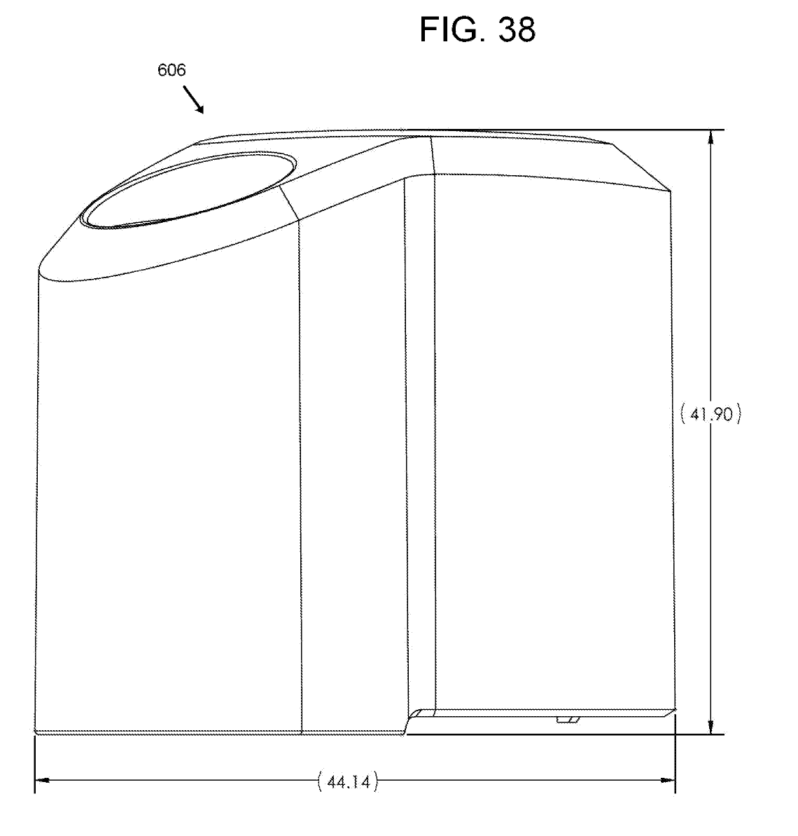

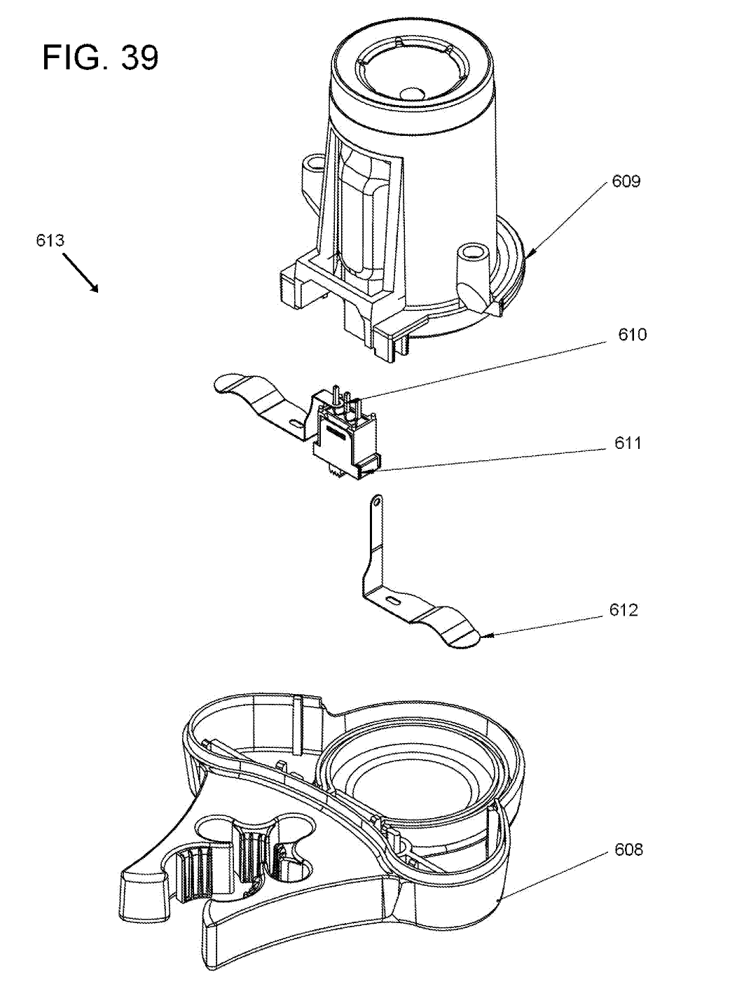

FIG. 38 shows an additional side view of the housing top 606. The housing assembly 606 has a possible length of 44.14 millimeters and a possible height of 41.9 millimeters.

FIG. 39 is a perspective view of an expanded subassembly of the bottom 613 of a housing assembly 601, showing individual components including a housing bottom 608, a reservoir subassembly 609, a battery contact B 610, an e-switch 500ASSPM2E 611, and a battery contact C 612. The e-switch 500ASSPM2E 611 and battery contact C 612 are configured to be ultrasonically welded together.

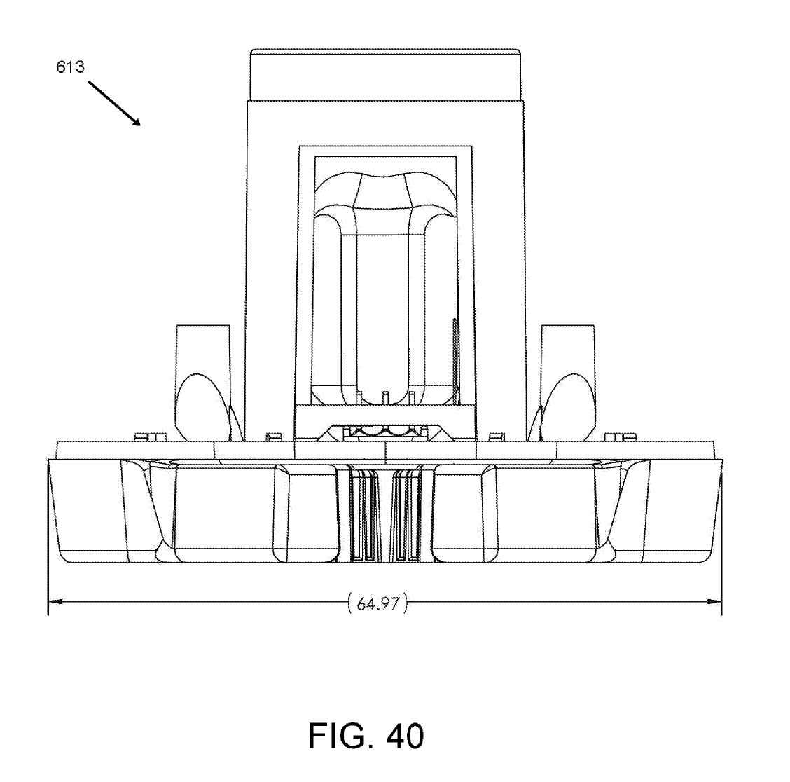

FIG. 40 shows a side view of the assembled bottom assembly 613. The housing bottom 613 has a possible width of 64.97 millimeters.

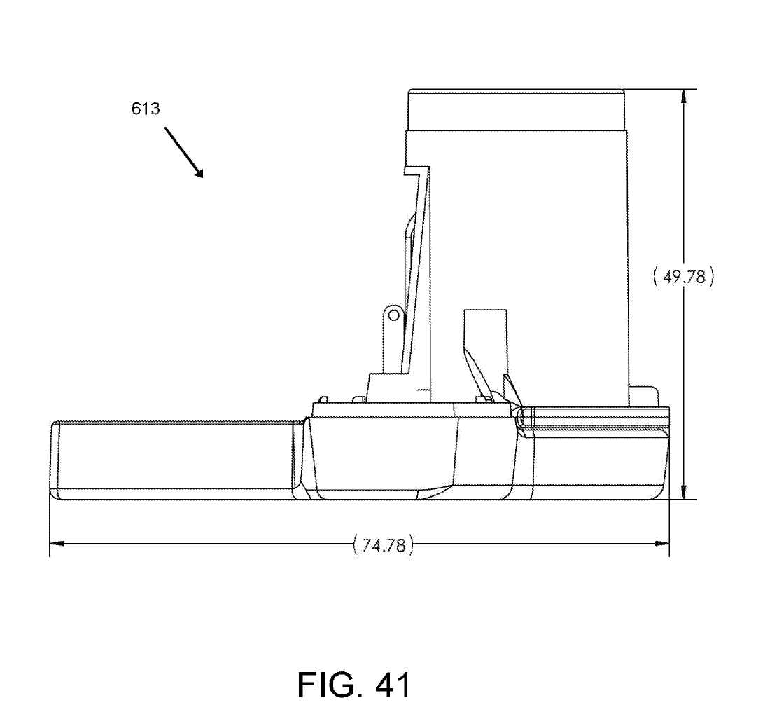

FIG. 41 shows an additional side view of the housing bottom assembly 613. The housing bottom 613 has a possible length of 74.78 millimeters and a possible height of 49.78 millimeters.

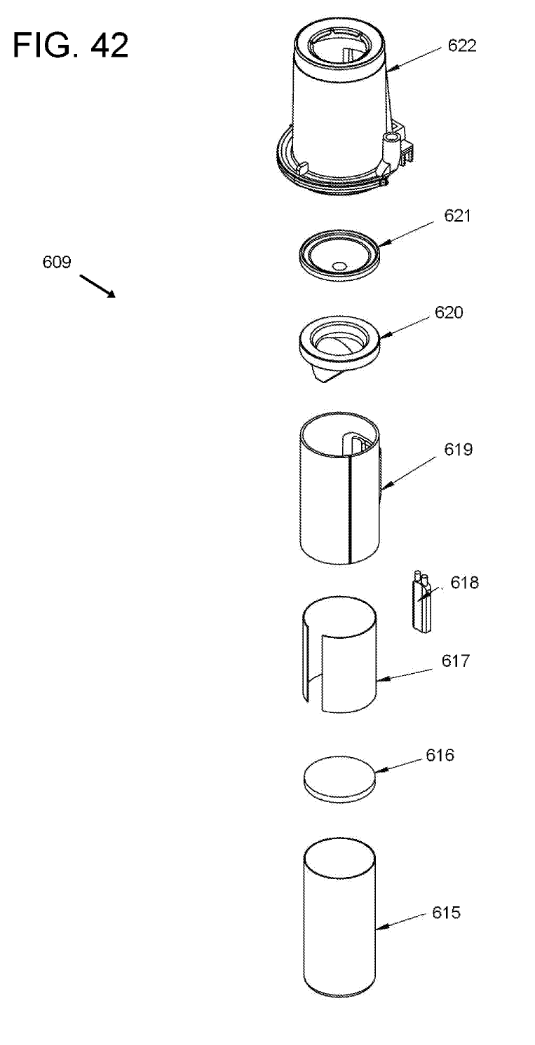

FIG. 42 is a perspective view of an expanded subassembly of a reservoir assembly 609, showing individual components including a reservoir 615, a reservoir sponge 616, a heater 617, a thermostat TB02-BB8D-055 618, a reservoir insulation 619, an inner seal 620, an outer seal 621, and a reservoir retainer 622. The reservoir sponge 616 is configured to be pressed into the bottom of the reservoir 615.

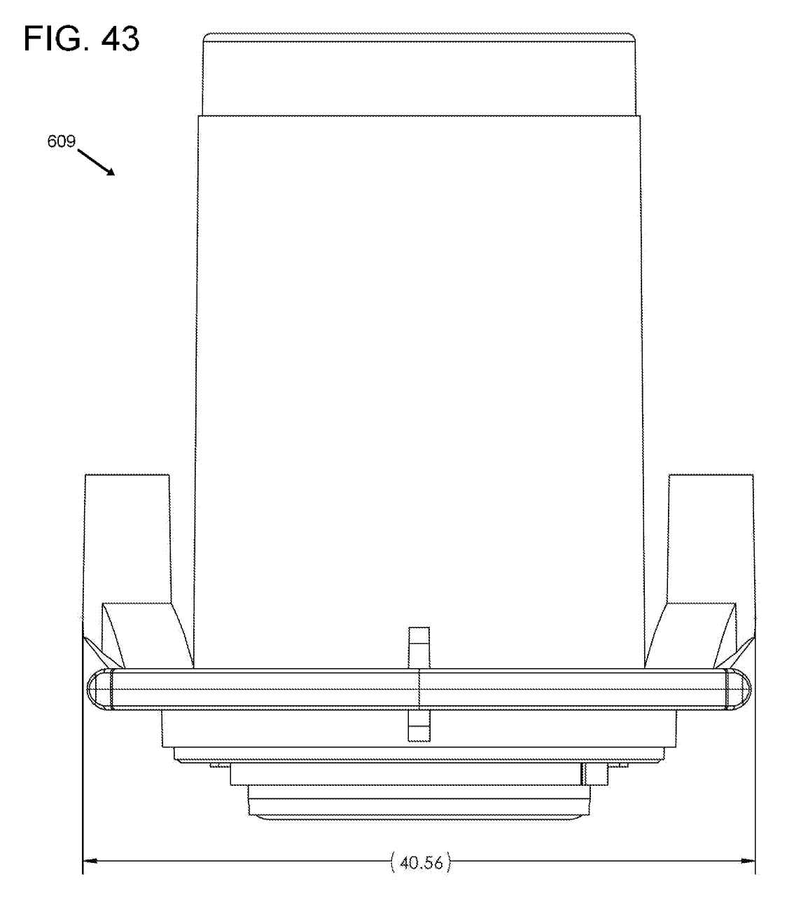

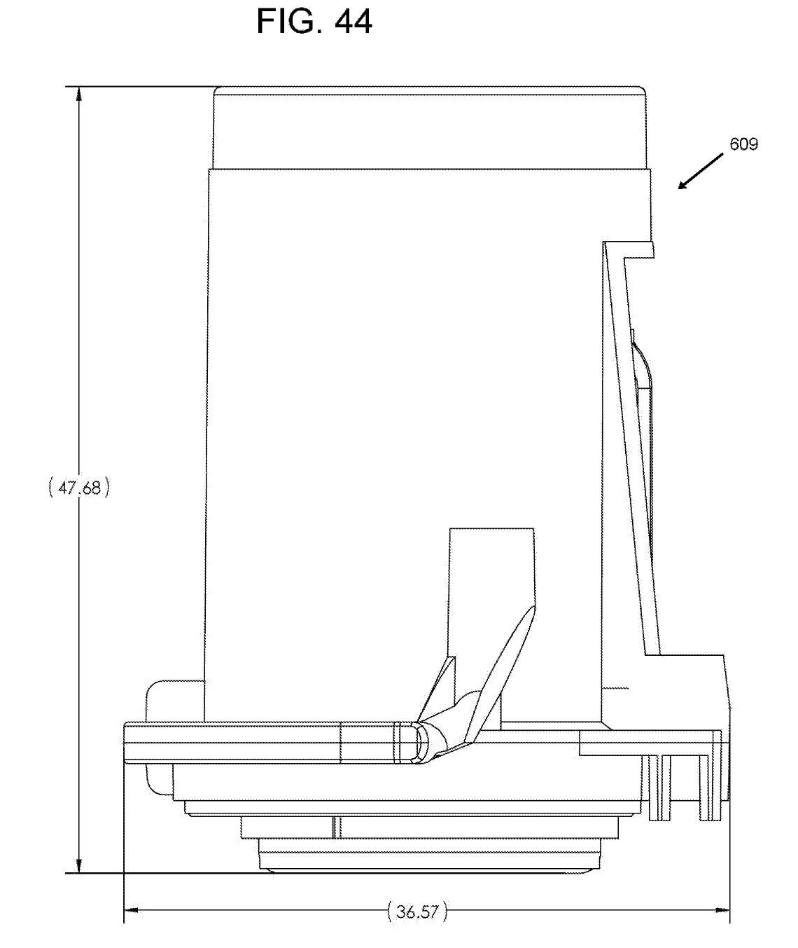

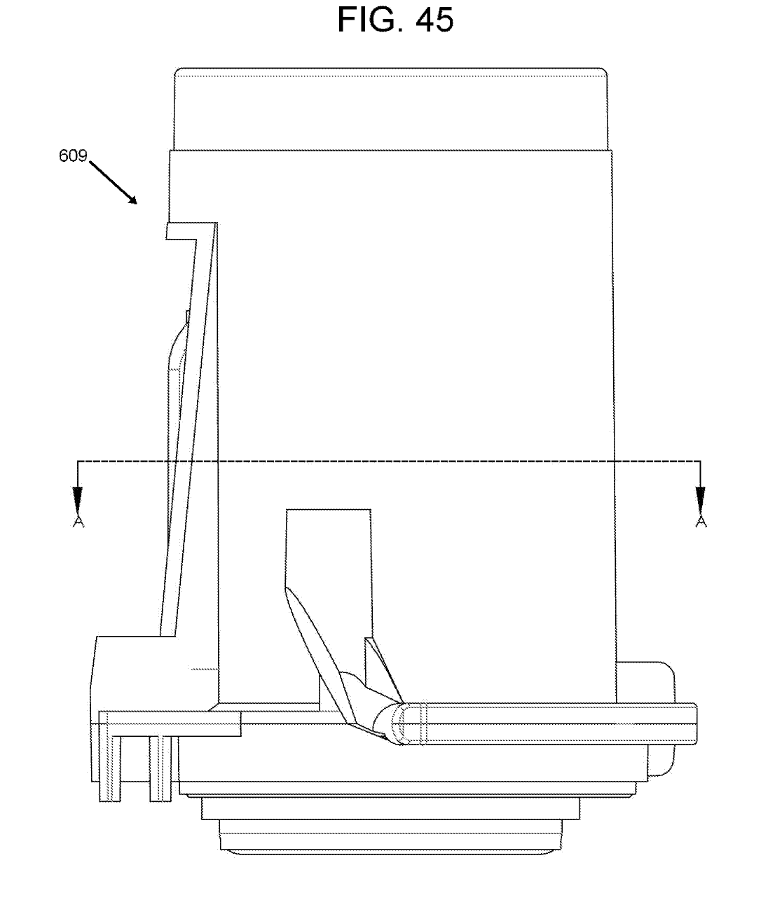

FIG. 43 shows a side view of the assembled reservoir subassembly 609. The reservoir assembly 609 has a possible width of 40.56 millimeters.

FIG. 44 shows an additional side view of the assembled reservoir subassembly 609. The reservoir assembly 609 has a possible length of 36.57 millimeters and a possible height of 47.68 millimeters.

FIG. 45 shows an assembled reservoir subassembly 609 with a line A-A to show where a cross-sectional cut is being taken.

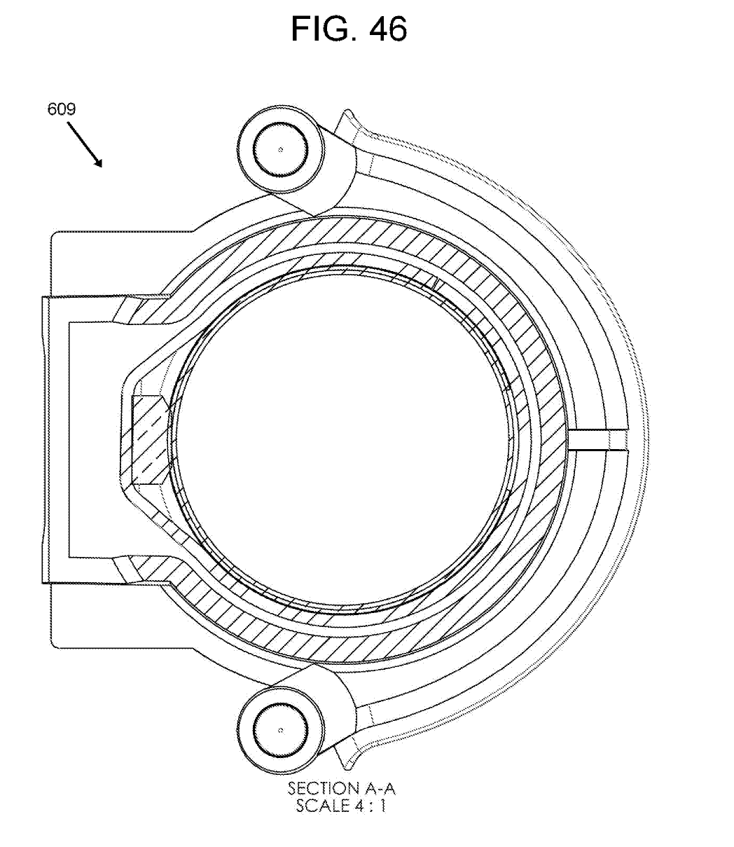

FIG. 46 shows the cross-section of the assembled reservoir subassembly 609 as shown in FIG. 45. The heater 617 should be attached to the reservoir 615 before the thermostat 618. The thermostat 618 should be attached to the outer surface of the heater 617, centered between the ends with the wire leads pointed toward the reservoir opening. The thermostat 618 should be adhered to the heater 617 with thermally conductive adhesive. The thermostat 618 should be wrapped with insulation 619, with the insulation split not located over the thermostat 618. The insulated reservoir should be aligned with the thermostat 618 centered in the vertical opening in the retainer housing.

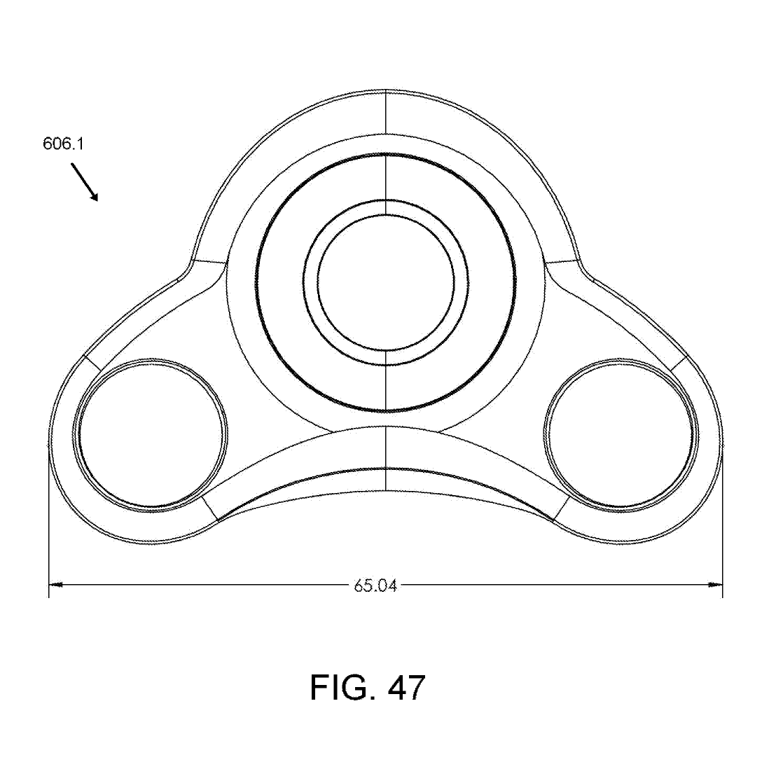

FIG. 47 shows a top view of a possible exemplification of a housing top 606.1. The housing top 606.1 has a possible width of 65.04 millimeters.

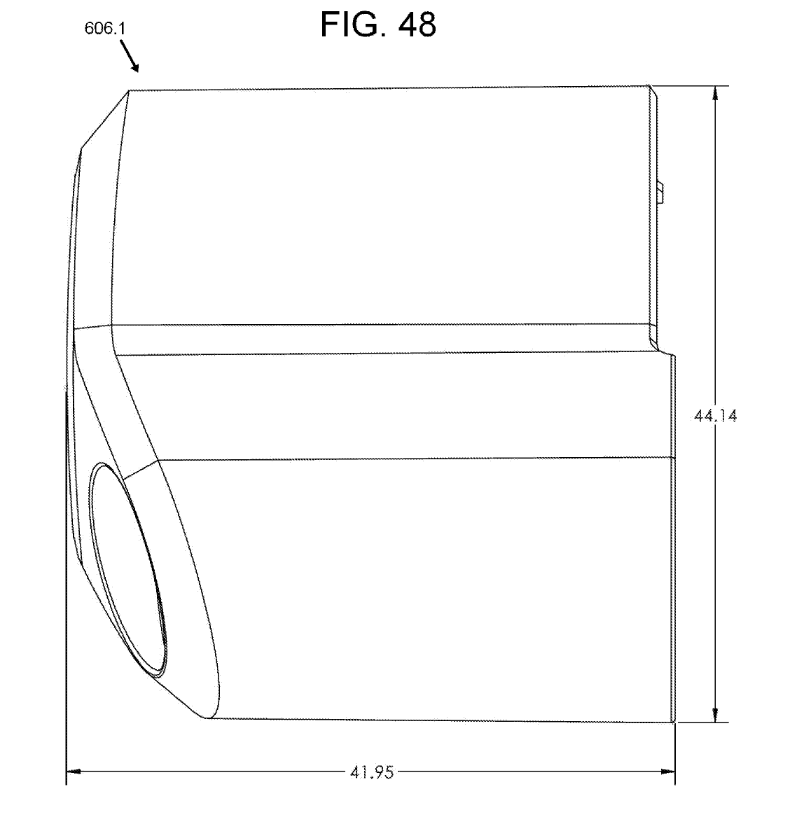

FIG. 48 shows a side view of the possible exemplification of the housing top 606.1, including a possible length of 41.95 millimeters and a possible height of 44.14 millimeters.

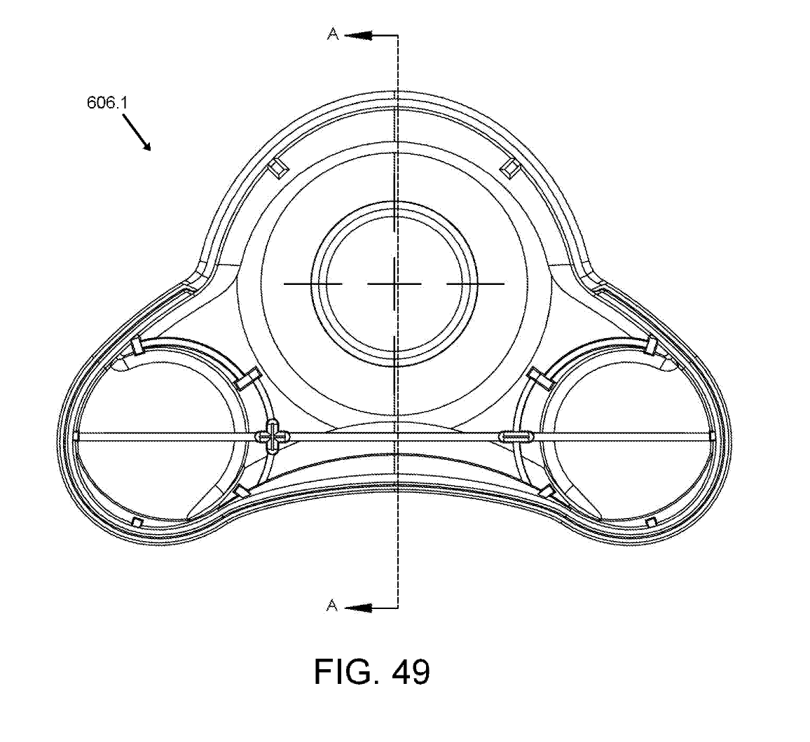

FIG. 49 shows a housing top 606.1 with a line A-A to show where a cross-sectional cut is being taken.

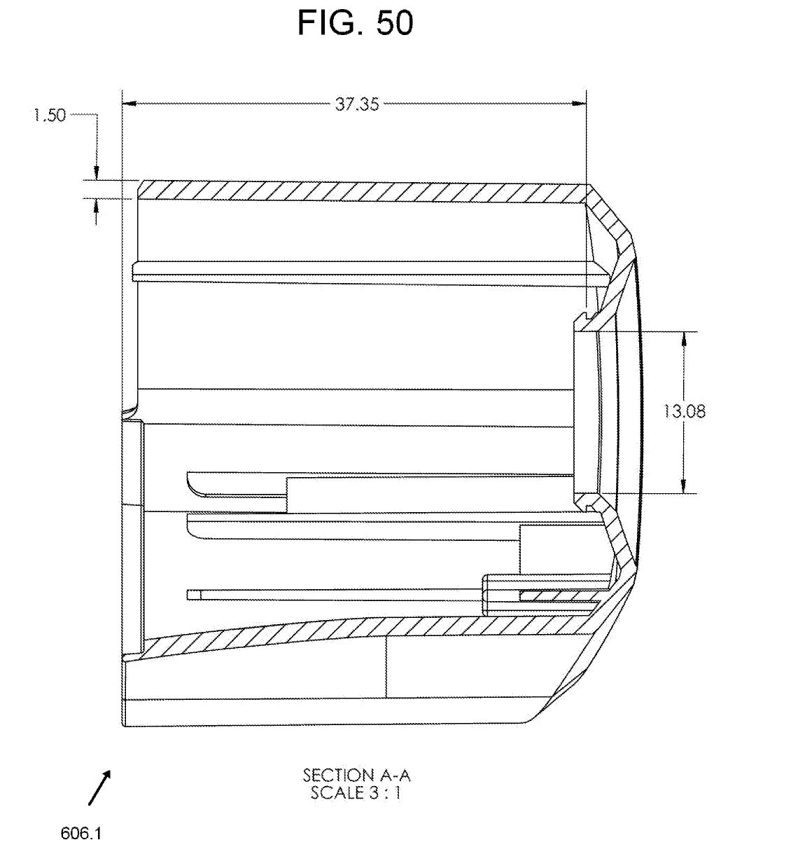

FIG. 50 shows the cross-section of the housing top 606.1 as shown in FIG. 49.

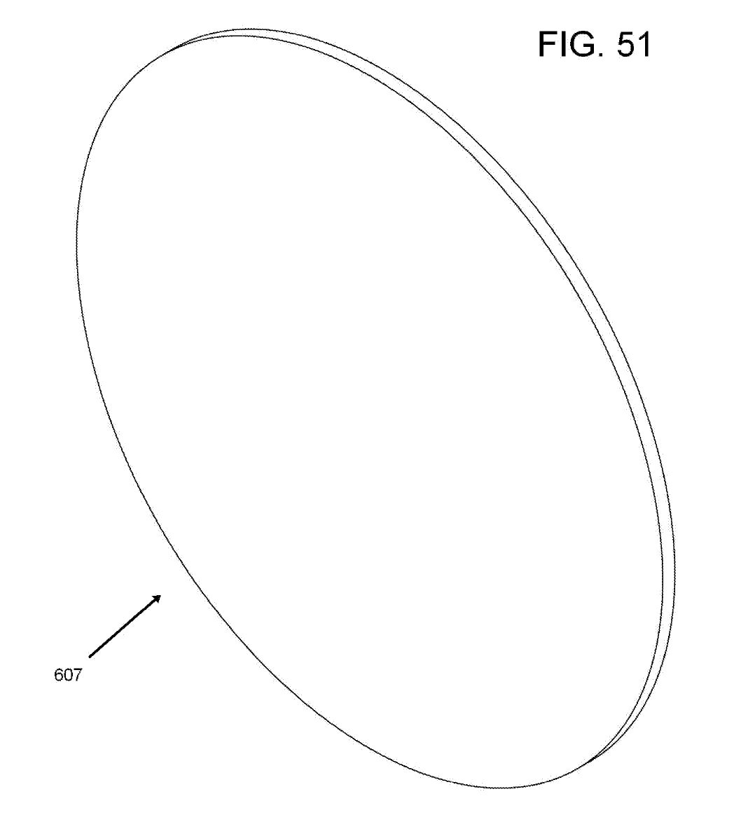

FIG. 51 is a perspective view of one possible exemplification of a microfiber patch 607 that may be adhered to a housing top of the present application.

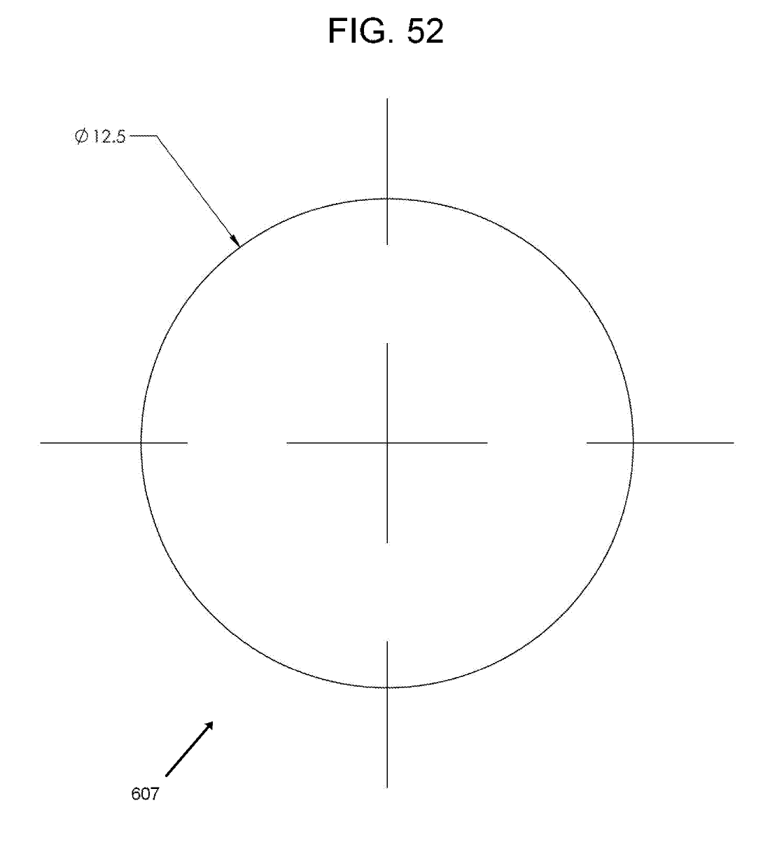

FIG. 52 shows a front view of the microfiber patch 607.

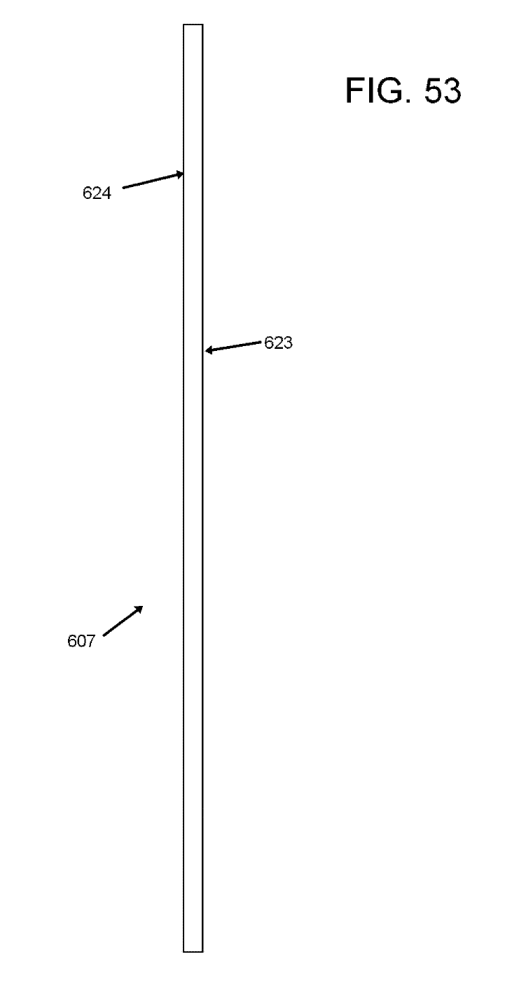

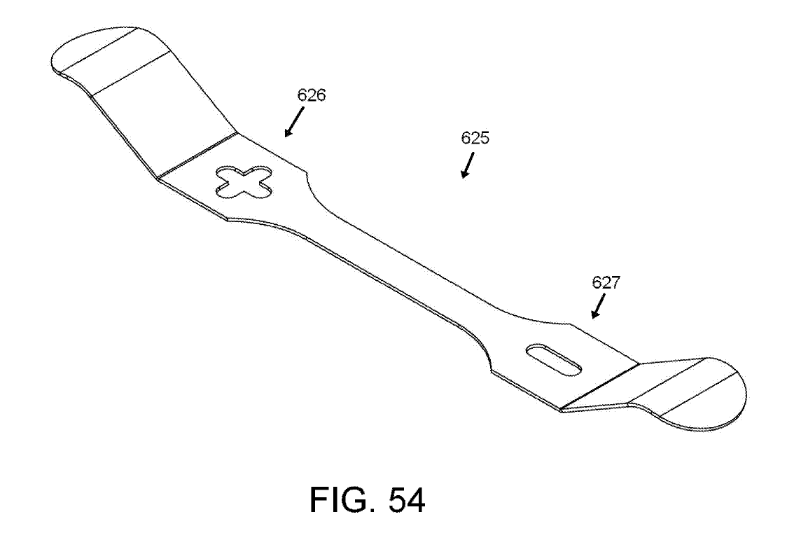

FIG. 53 shows a side view of the microfiber patch 607. The microfiber patch 607 comprises two sides. One side 623 is configured to be adhered to the housing top, and the opposite site 624 is configured to be exposed.

FIG. 54 shows a first battery contact 625. The battery contact 625 may be comprised of beryllium copper C172 alloy, 1/2-hard heat transfer after forming to 1/2 HT temper, with a finish of electroless nickel plate. The battery contact 625 has a positive contact point 626 and a negative contact point 627, so that it will contact two batteries during use.

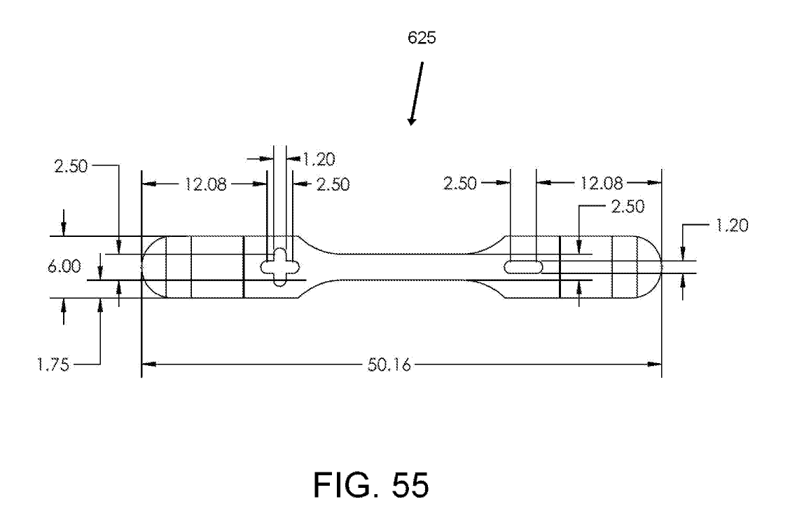

FIG. 55 is a top view of the battery contact 625.

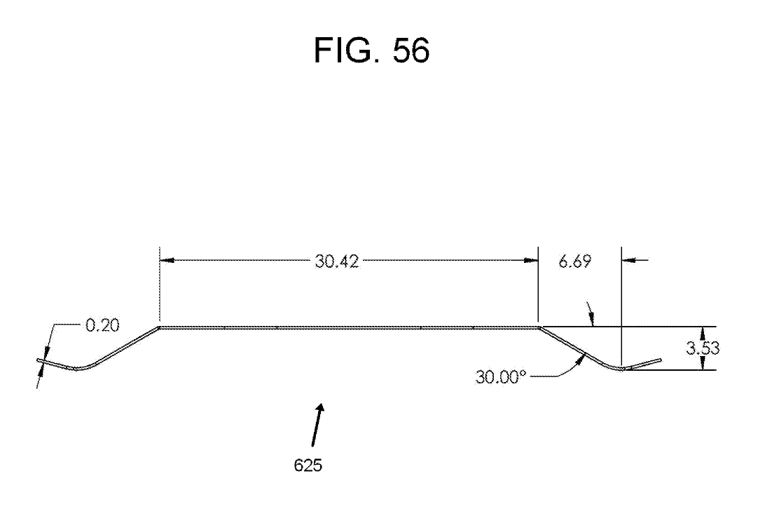

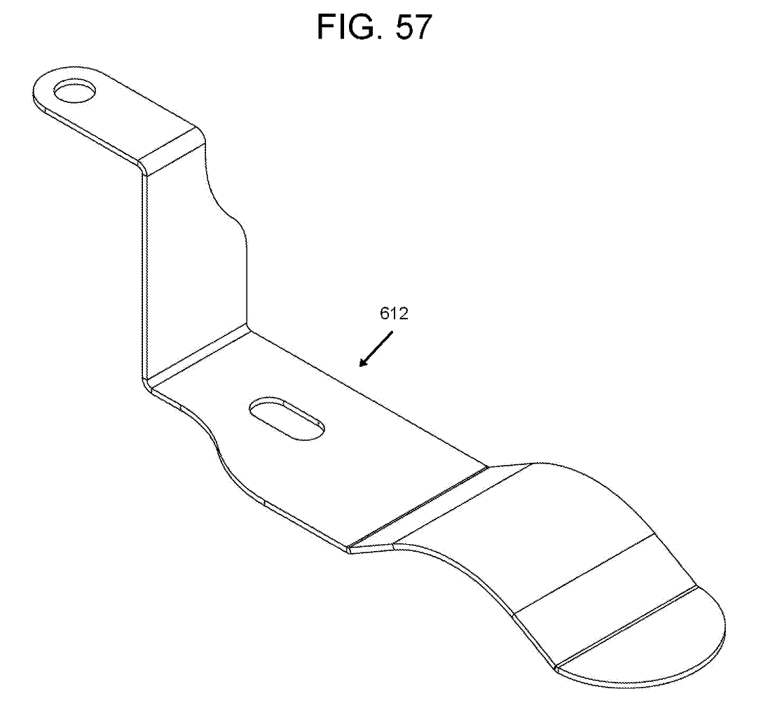

FIG. 56 is a side view of the battery contact 625.

FIG. 57 shows a perspective view of battery contact 612. The battery contact 612 may be comprised of beryllium copper C172 alloy, 1/2-hard heat transfer after forming to 1/2 HT temper, with a finish of electroless nickel plate.

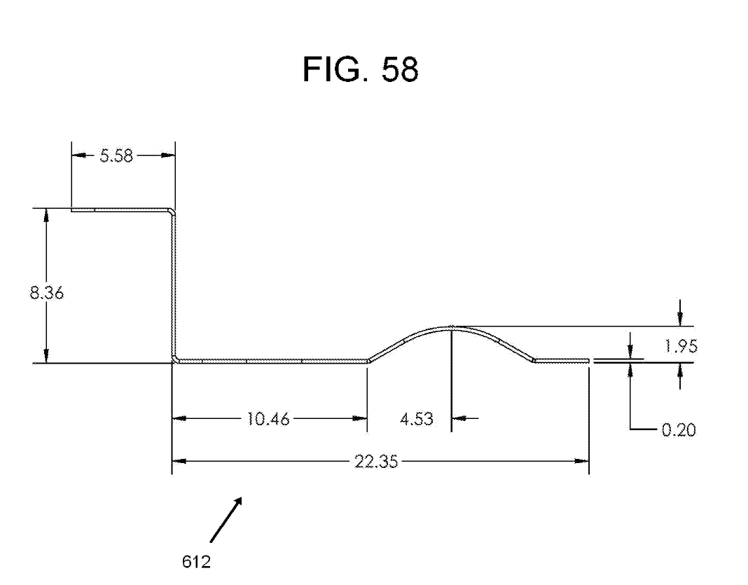

FIG. 58 shows a side view of the battery contact 612.

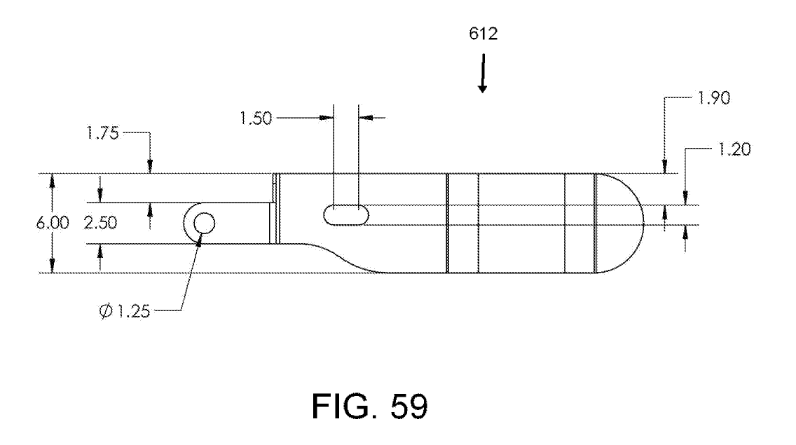

FIG. 59 shows a top view of the battery contact 612.

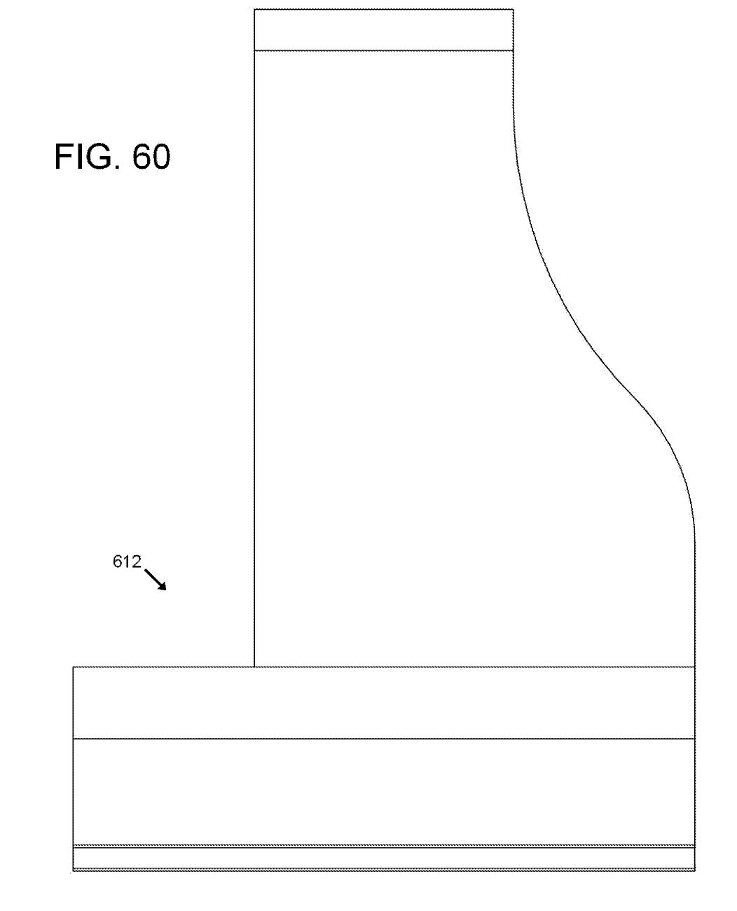

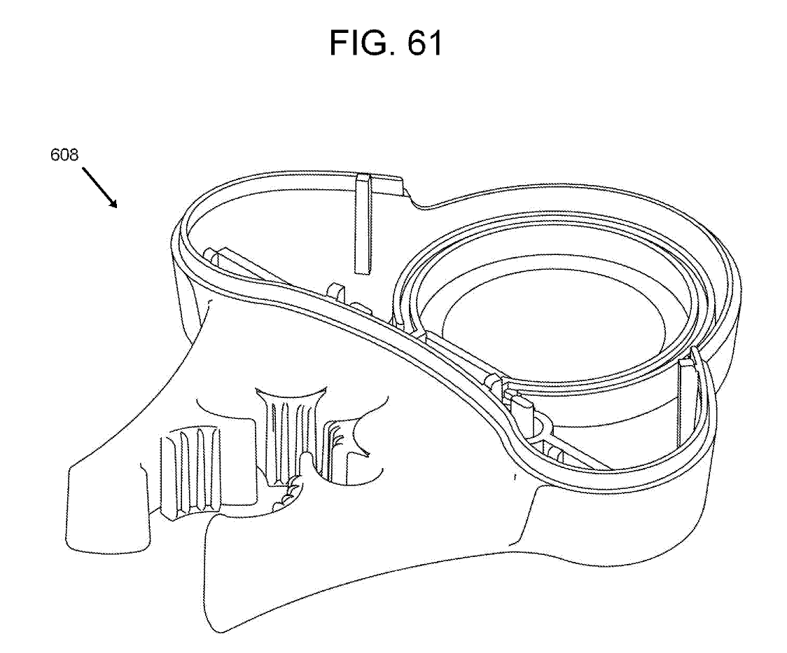

FIG. 60 shows a different side view of the battery contact 612.

FIG. 61 shows a perspective view of a housing bottom 608.

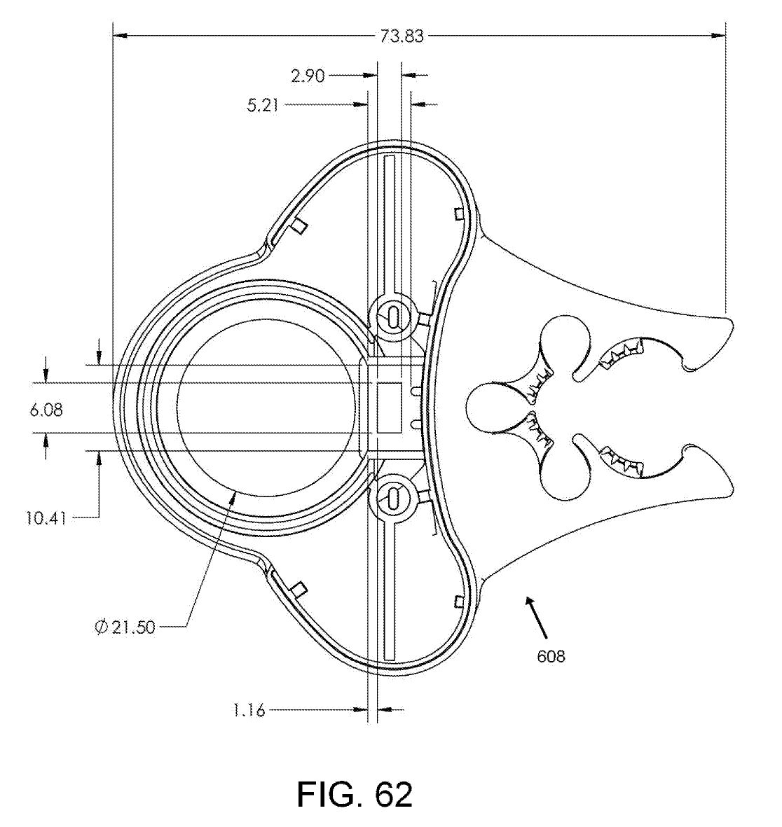

FIG. 62 shows a top view of the housing bottom 608. Some areas of the housing bottom 608 may be overmolded with textin Rx170A.

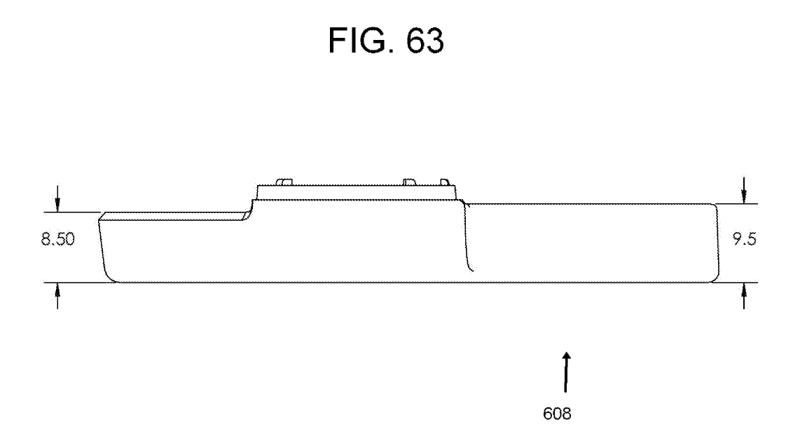

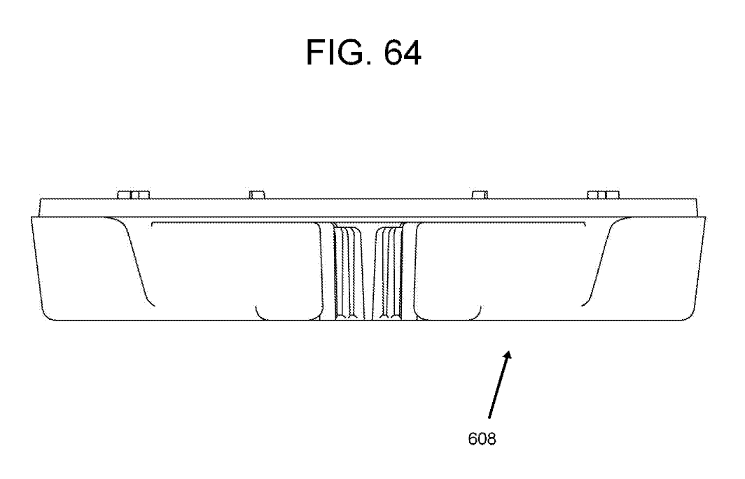

FIG. 63 shows a side view of the housing bottom 608.

FIG. 64 shows a different side view of the housing bottom 608.

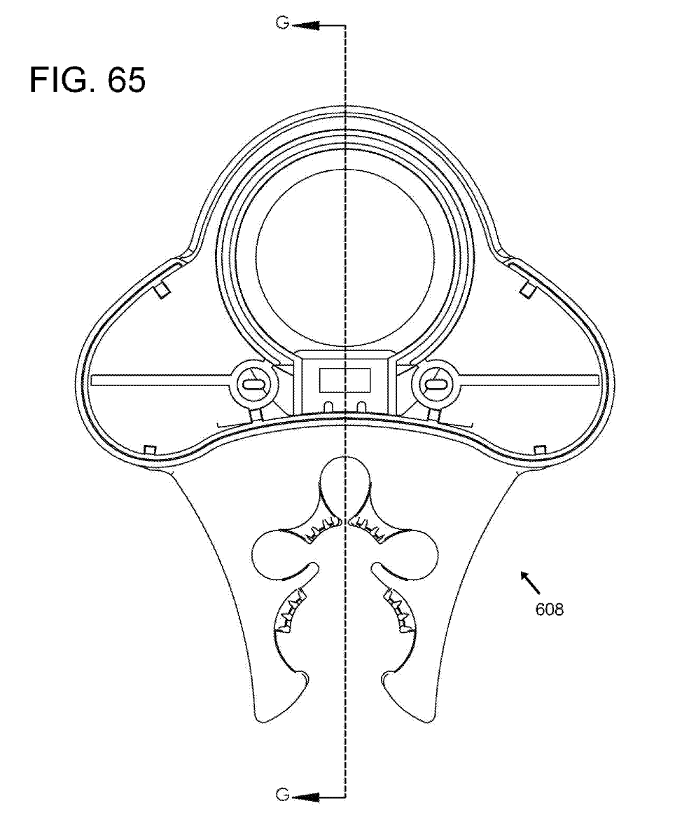

FIG. 65 shows a top view of the housing bottom 608 with a line G-G to show where a cross-sectional cut is being taken.

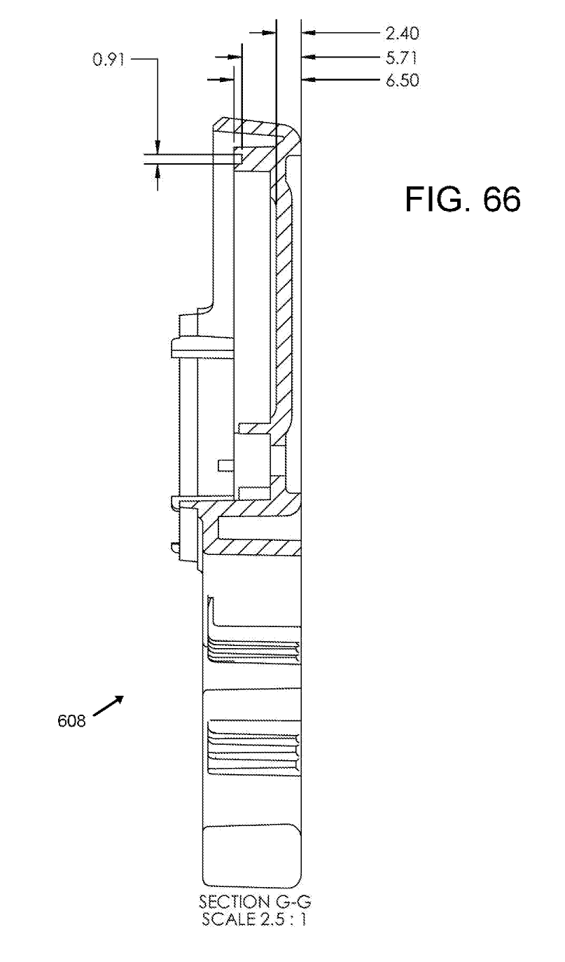

FIG. 66 shows the cross-section of the housing bottom 608 as shown in FIG. 65.

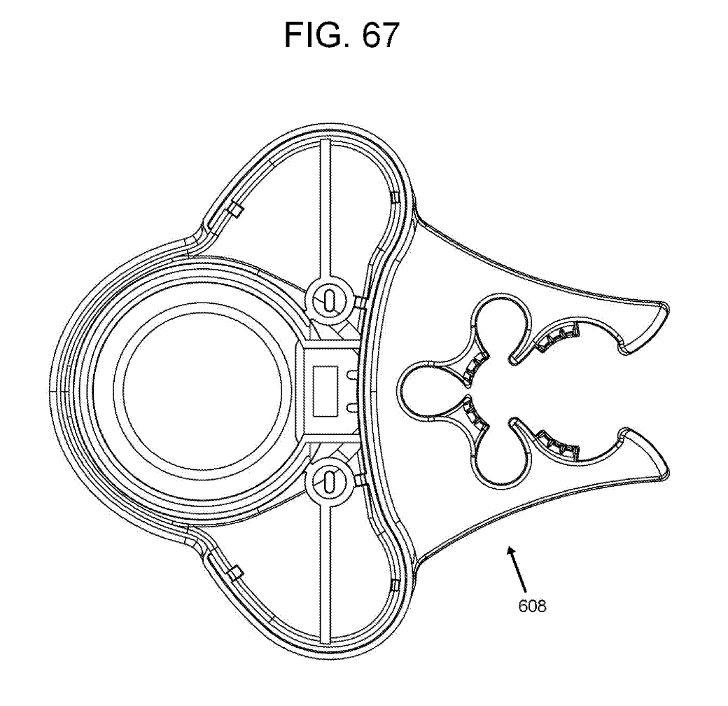

FIG. 67 shows another top view of the housing bottom 608.

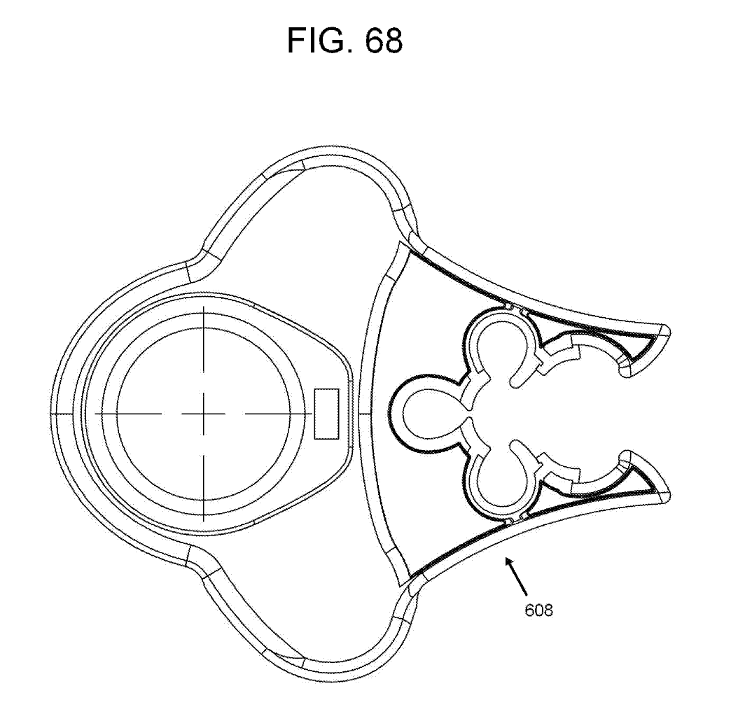

FIG. 68 shows the bottom view of the housing bottom 608.

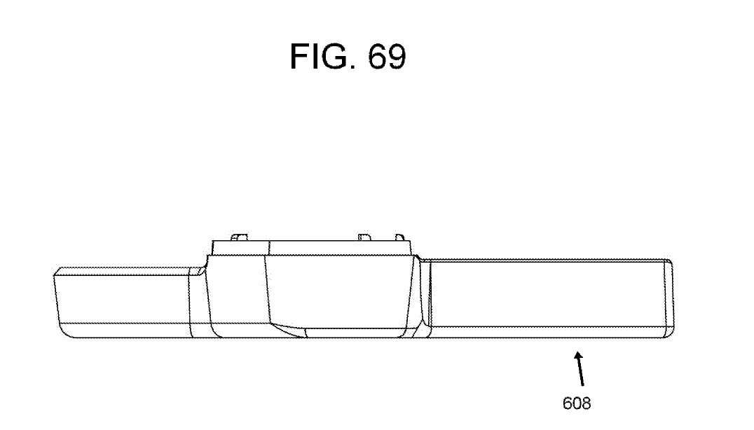

FIG. 69 shows a side view of the housing bottom 608.

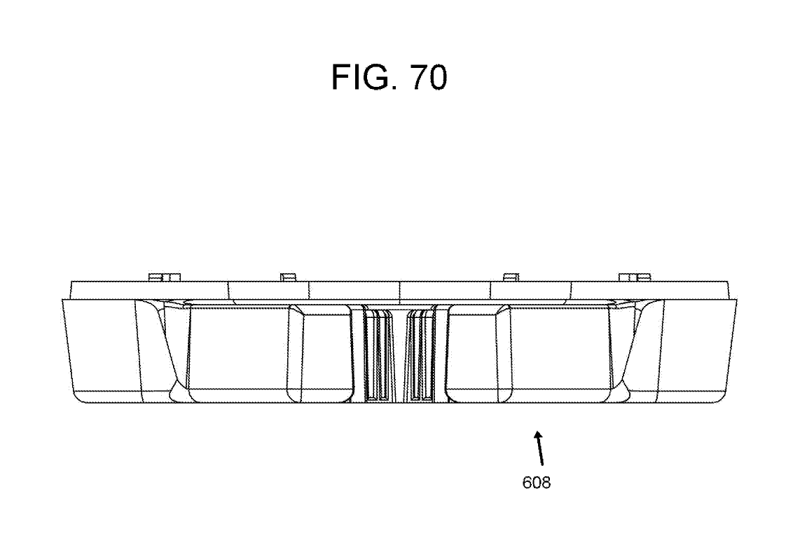

FIG. 70 shows a front view of the housing bottom 608.

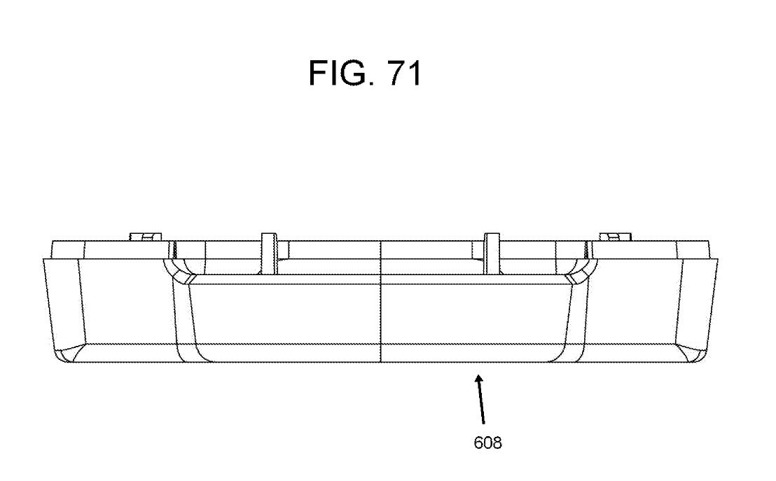

FIG. 71 shows a back view of the housing bottom 608.

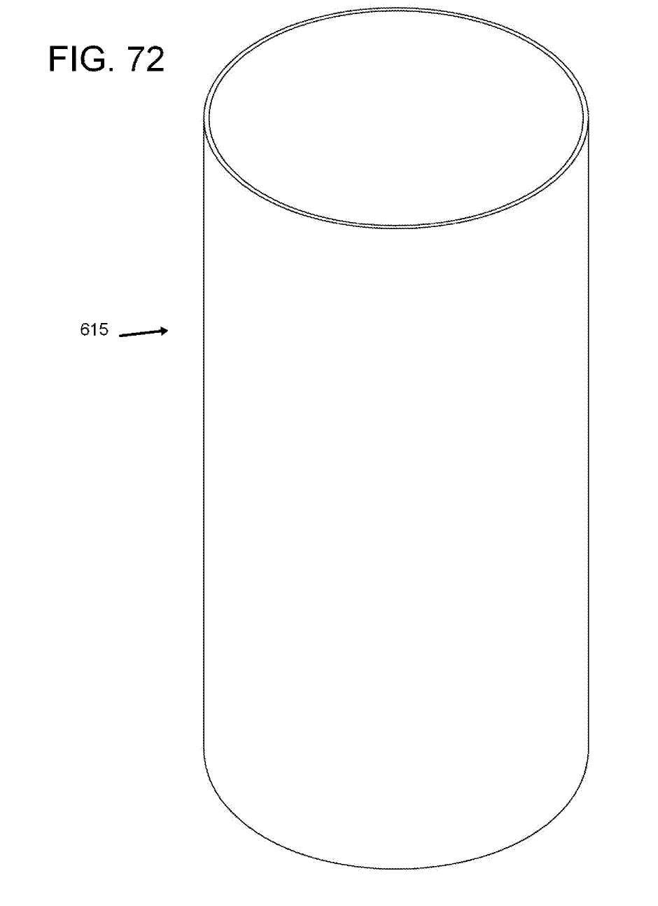

FIG. 72 shows a perspective view of a reservoir 615.

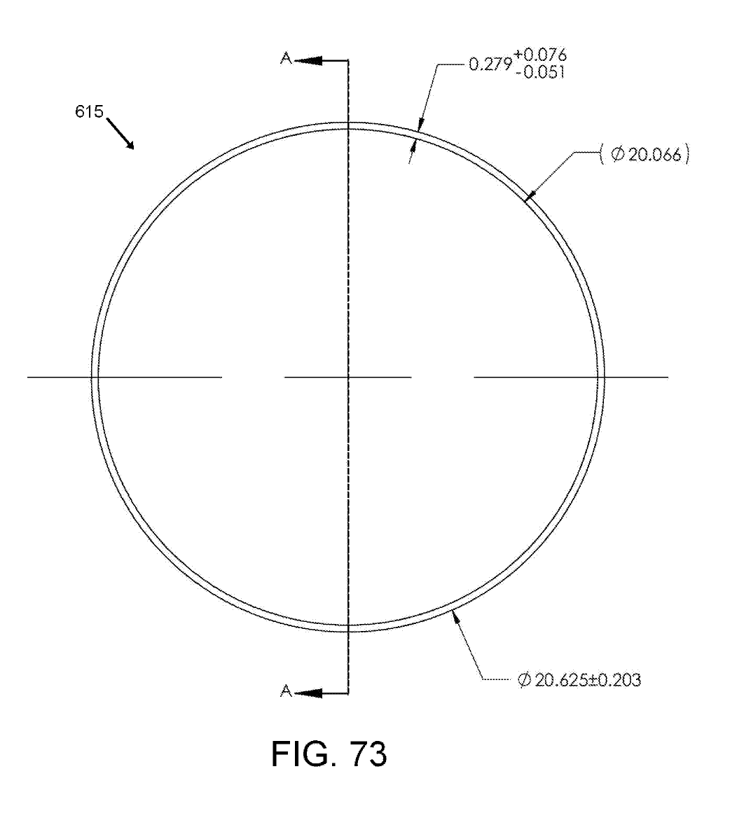

FIG. 73 shows a top view of the reservoir 615 with a line A-A to show where a cross-sectional cut is being taken.

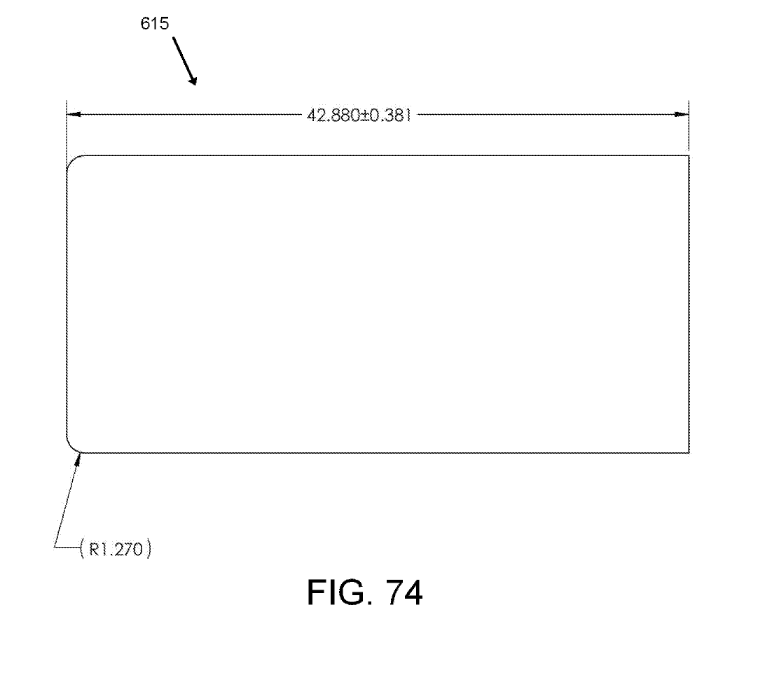

FIG. 74 shows a side view of the reservoir 615.

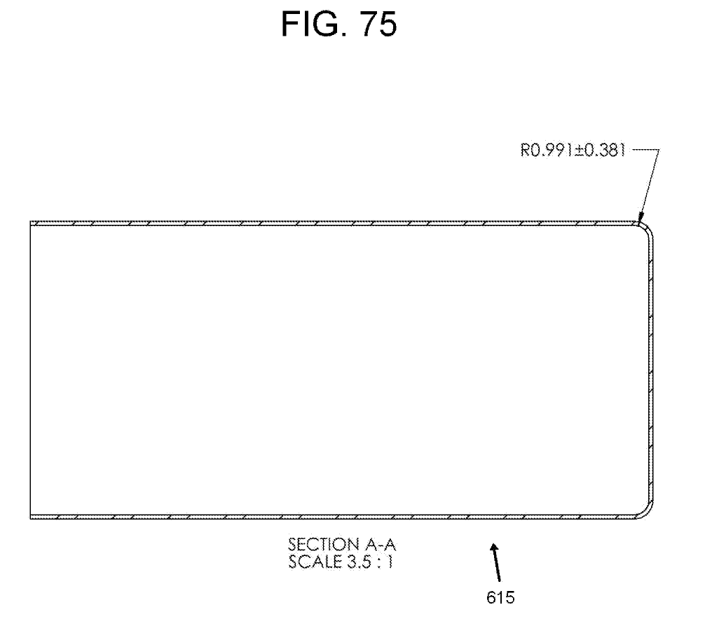

FIG. 75 shows a cross-section of the reservoir 615 as shown in FIG. 73.

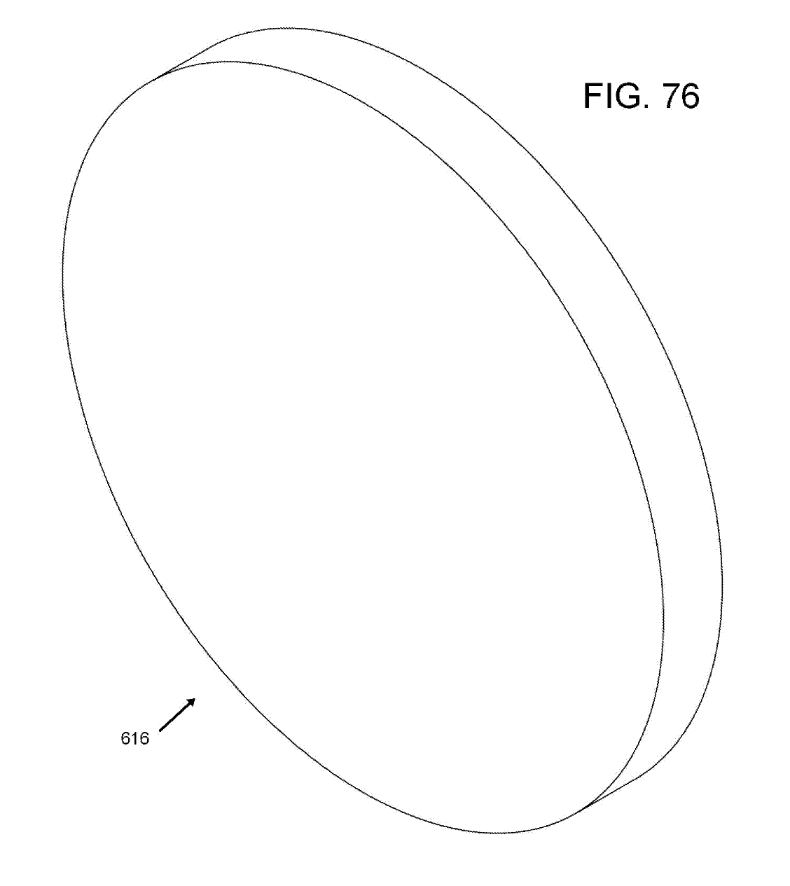

FIG. 76 is a perspective view of a reservoir sponge 616.

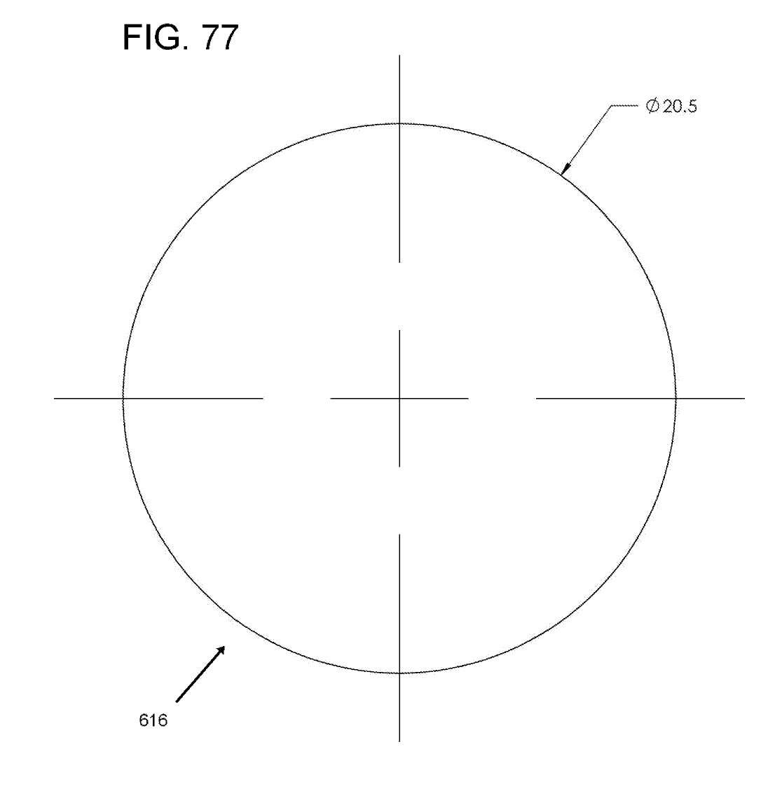

FIG. 77 shows a front view of the reservoir sponge 616.

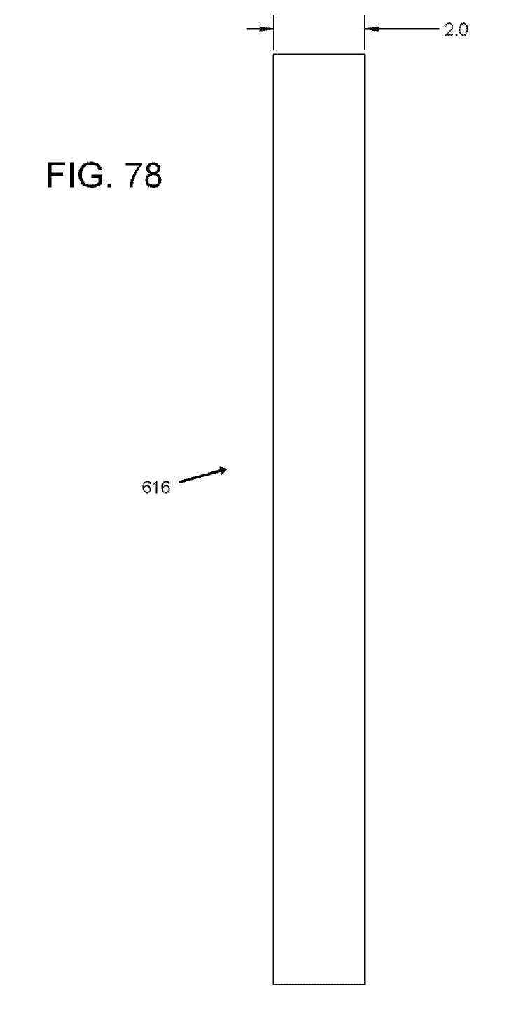

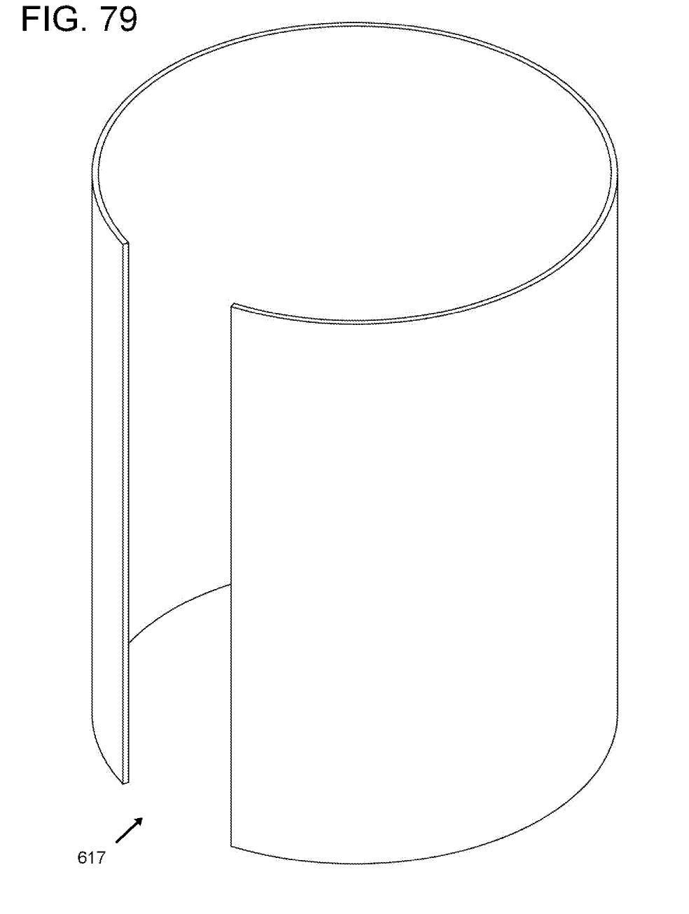

FIG. 78 shows a side view of the reservoir sponge 616.

FIG. 79 shows a perspective view of a heater 617.

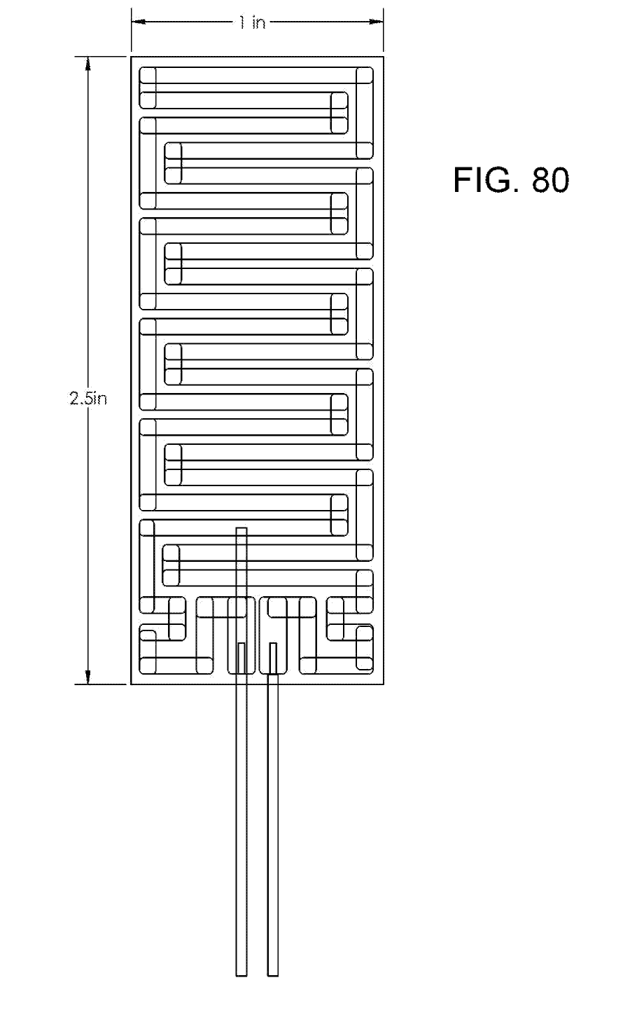

FIG. 80 shows the circuitry inside the heater 617.

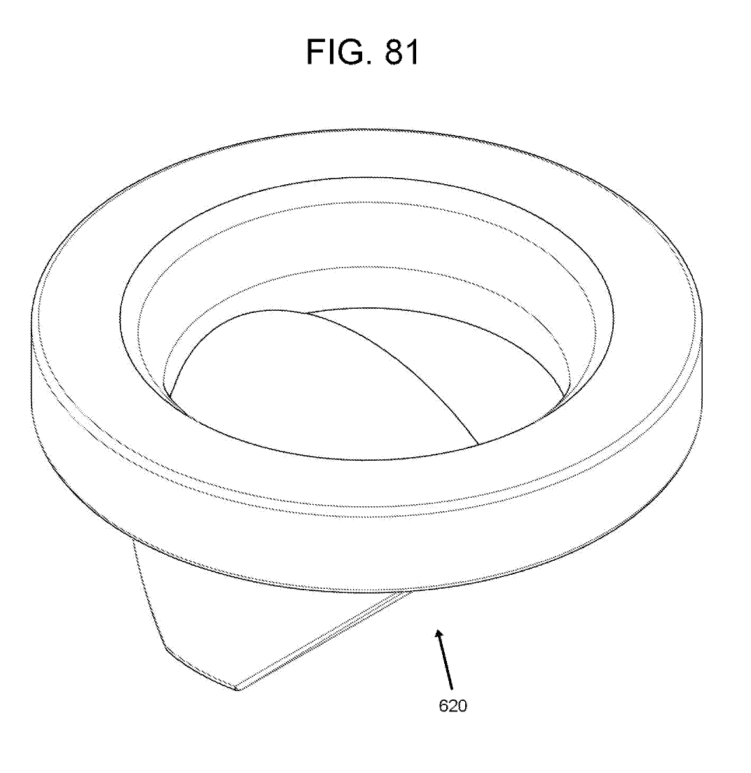

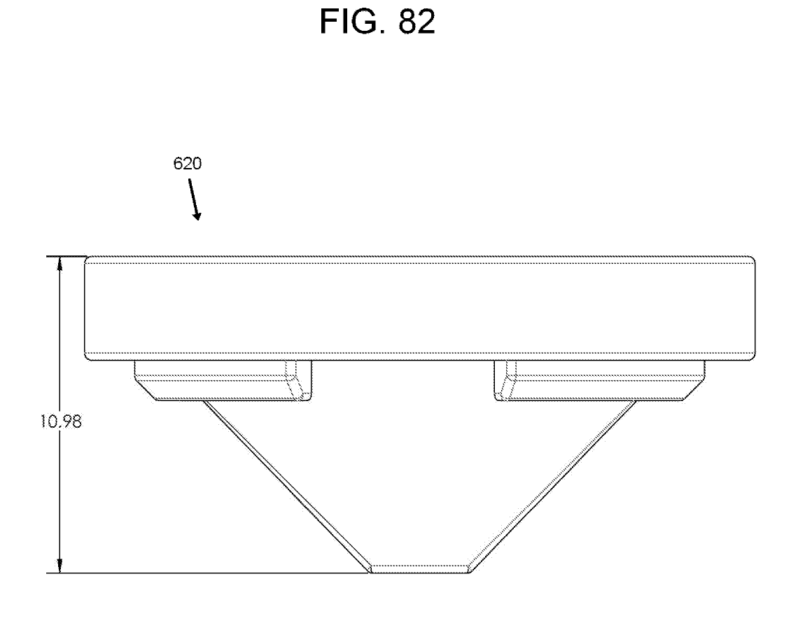

FIG. 81 is a perspective view of an inner seal 620.

FIG. 82 shows a side view of the inner seal 620.

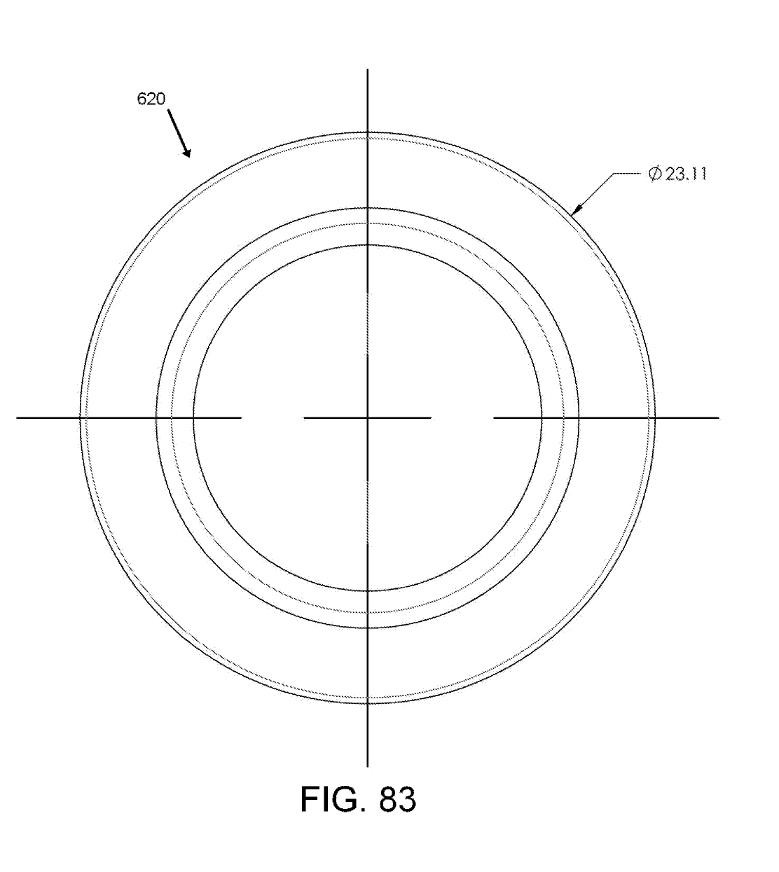

FIG. 83 shows a top view of the inner seal 620.

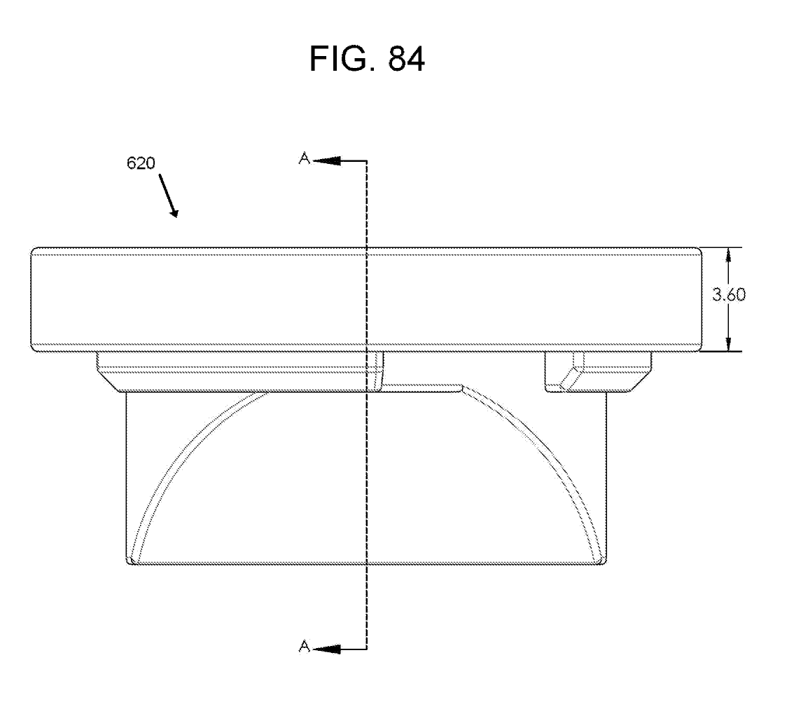

FIG. 84 shows another side view of the inner seal 620. With a line A-A to show where a cross-sectional cut is being taken.

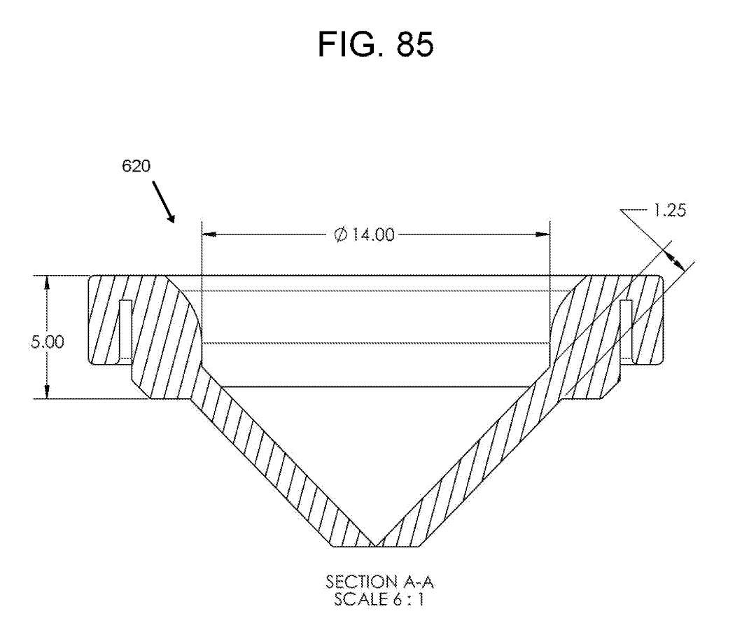

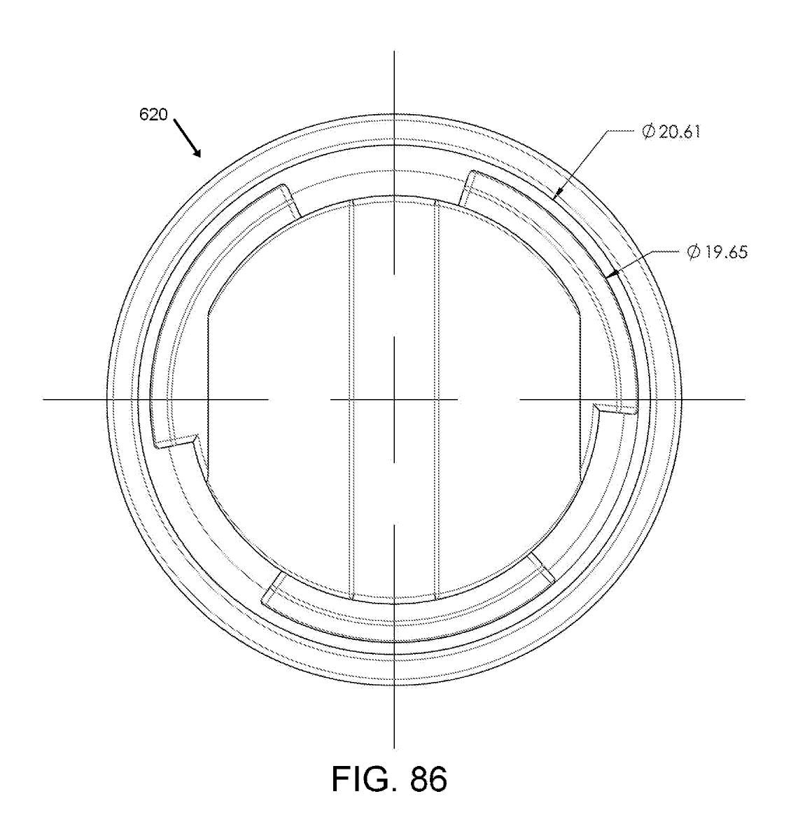

FIG. 85 shows a cross-section of the inner seal 620 as shown in FIG. 84.

FIG. 86 shows a bottom view of the inner seal 620.

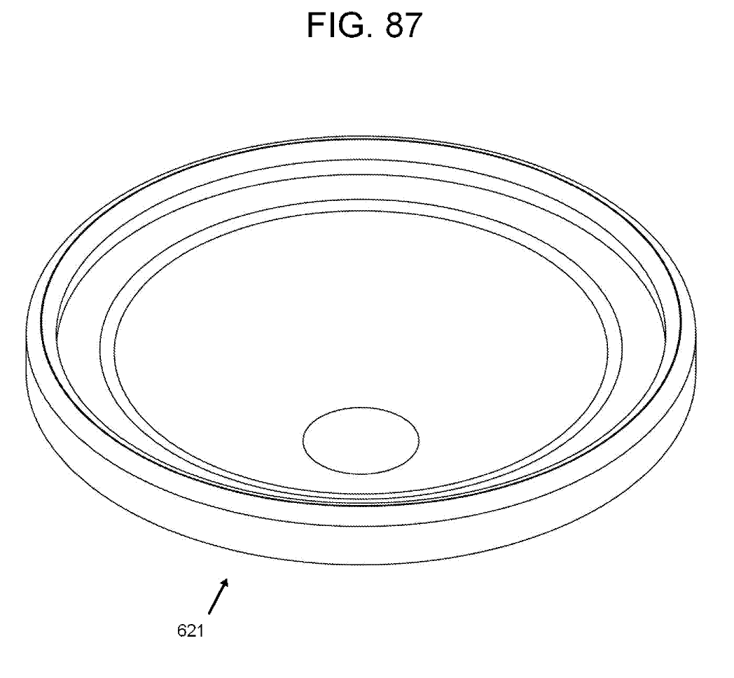

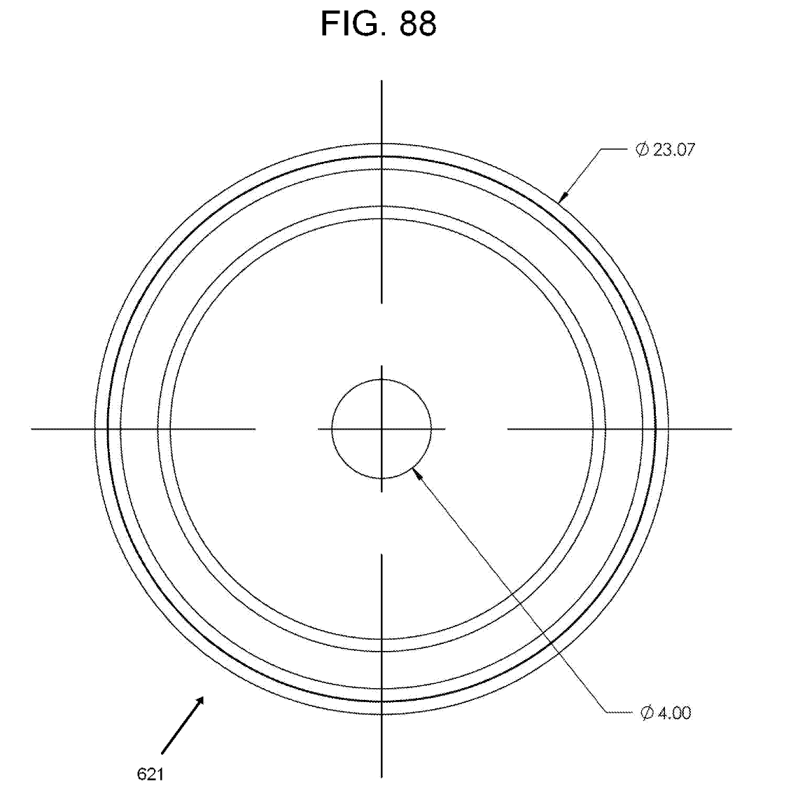

FIG. 87 is a perspective view of an outer seal 621.

FIG. 88 shows a top view of the outer seal 621.

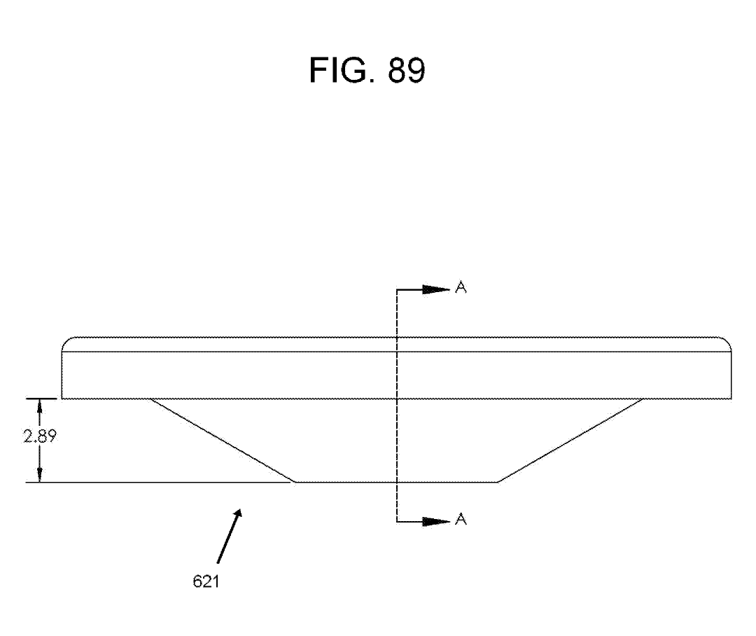

FIG. 89 shows a side view of the outer seal 621, with a line A-A to show where a cross section is taken.

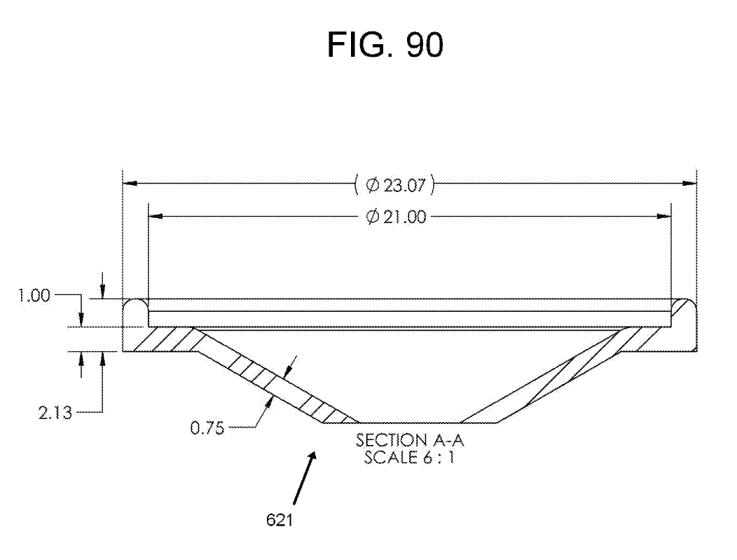

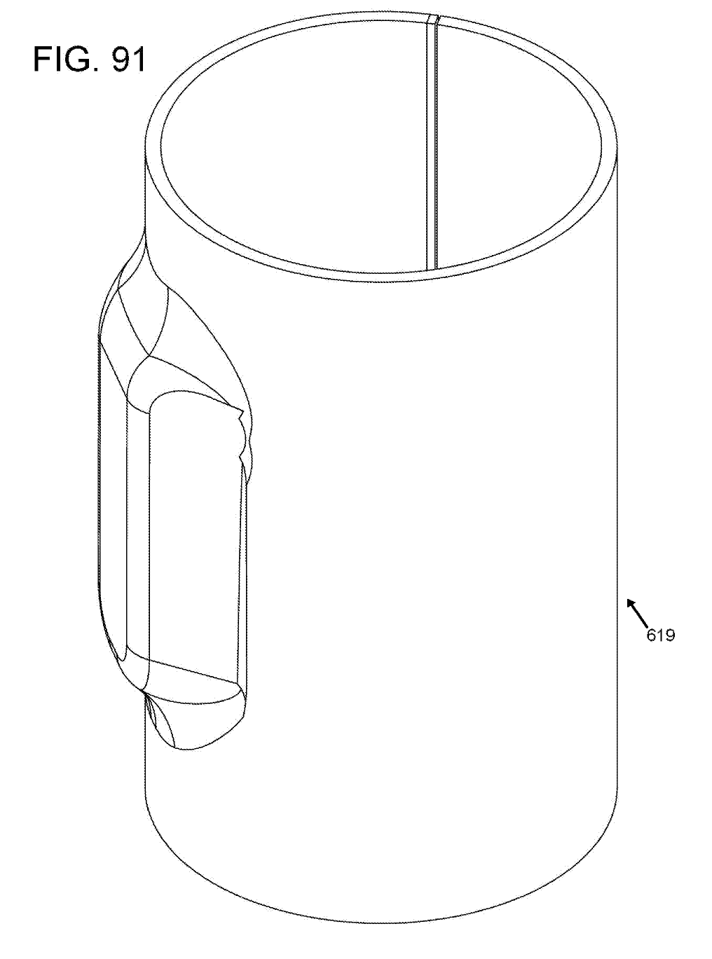

FIG. 90 shows the cross section of the outer seal 621, as shown in FIG. 89.

FIG. 91 is a perspective view of a reservoir installation 628, that is, an installed configuration 628. The material, in one possible exemplification a silicone tape, may lay over the thermostat 618.

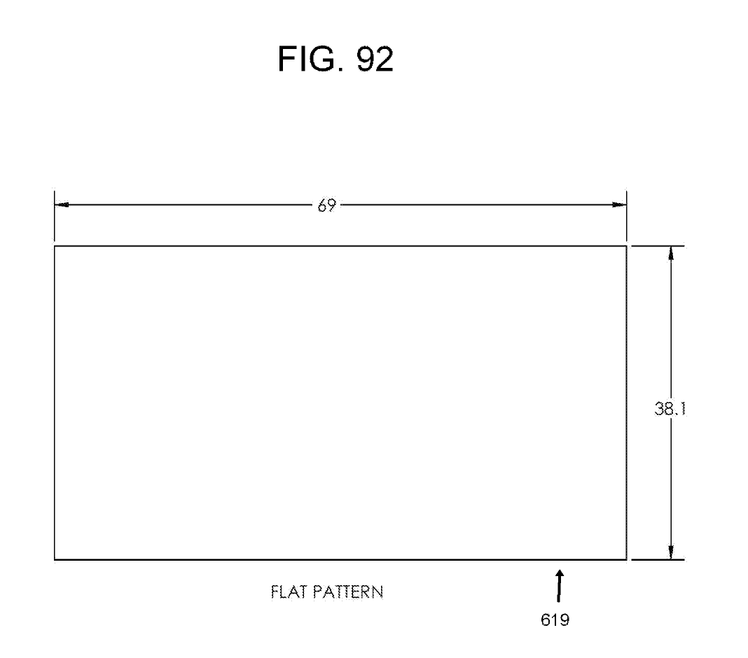

FIG. 92 shows a top view of reservoir insulation 619 in a flat pattern. Adhesive may be on one side of the reservoir insulation 619. The reservoir insulation 619 may have a width of 69 millimeters and a height of 38.1 millimeters.

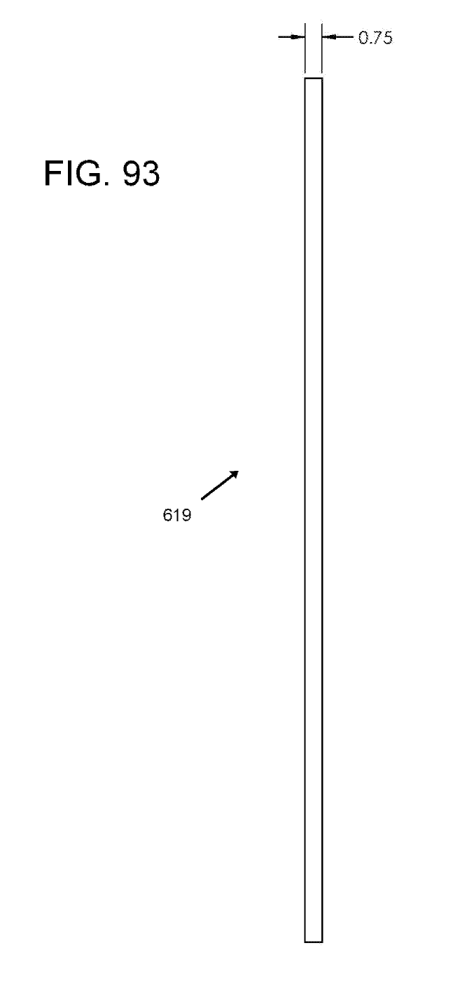

FIG. 93 shows a side view of the reservoir insulation 619. The reservoir insulation 619 may have a thickness of 0.75 millimeters.

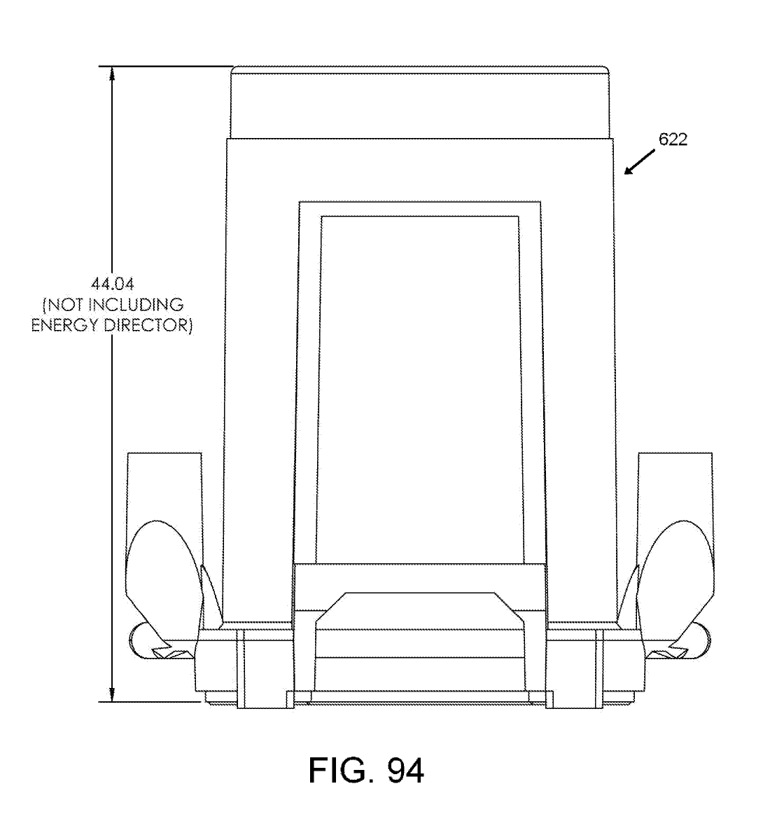

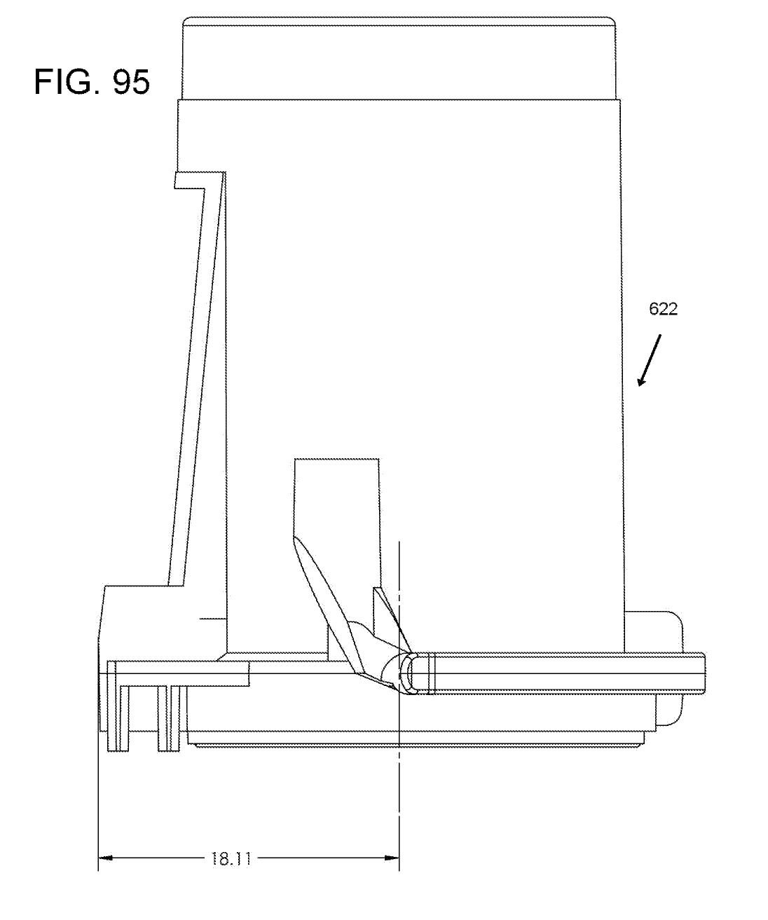

FIG. 94 shows one possible side view of a reservoir retainer 622.

FIG. 95 shows another possible side view of the reservoir retainer 622.

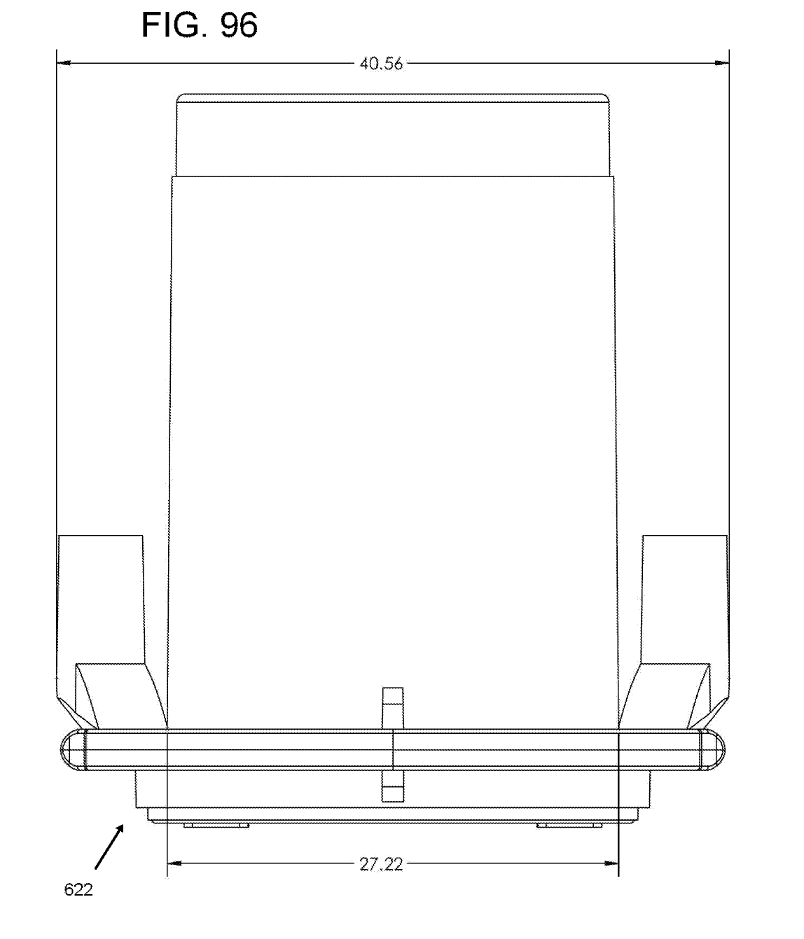

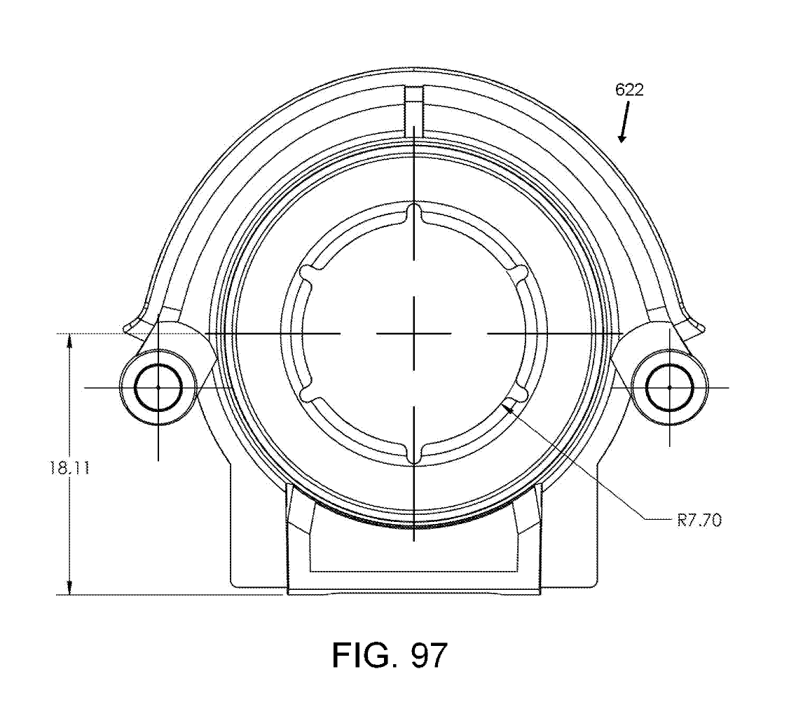

FIG. 96 shows another possible side view of the reservoir retainer 622.

FIG. 97 shows a top/bottom view of the reservoir retainer 622.

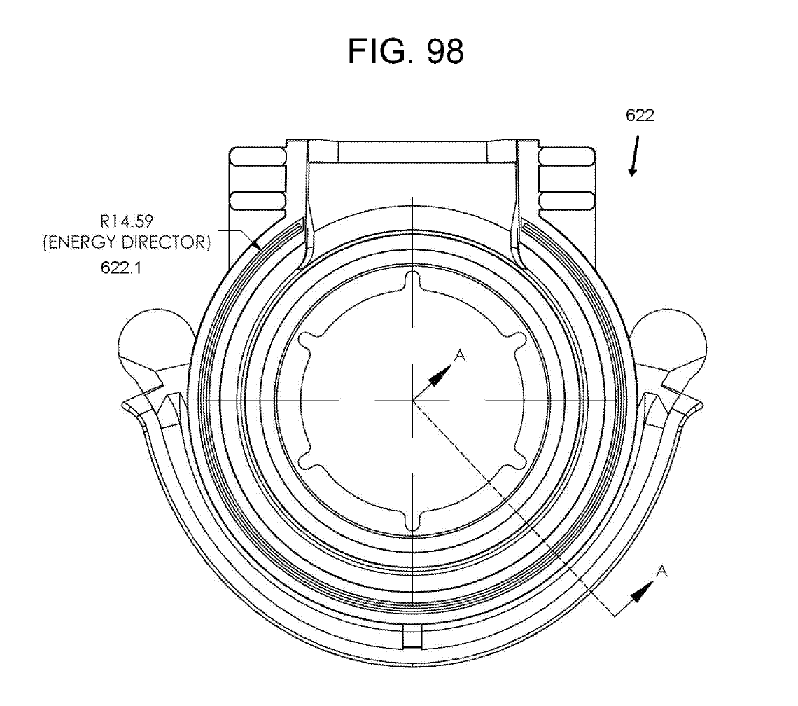

FIG. 98 shows a top/bottom view of the reservoir retainer 622 including an energy director 622.1, with a line A-A to show where a cross section is taken.

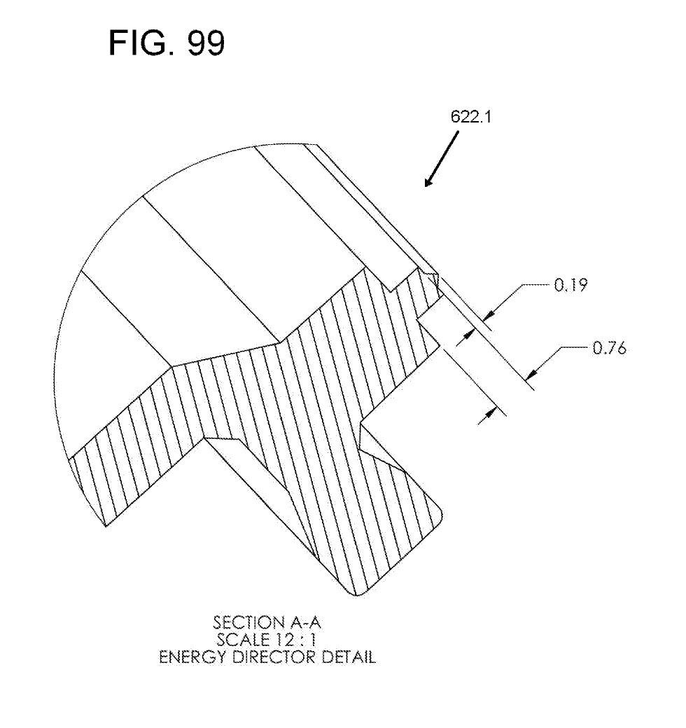

FIG. 99 shows the cross section of the energy director 622.1 of the reservoir retainer 622 as shown in FIG. 98.

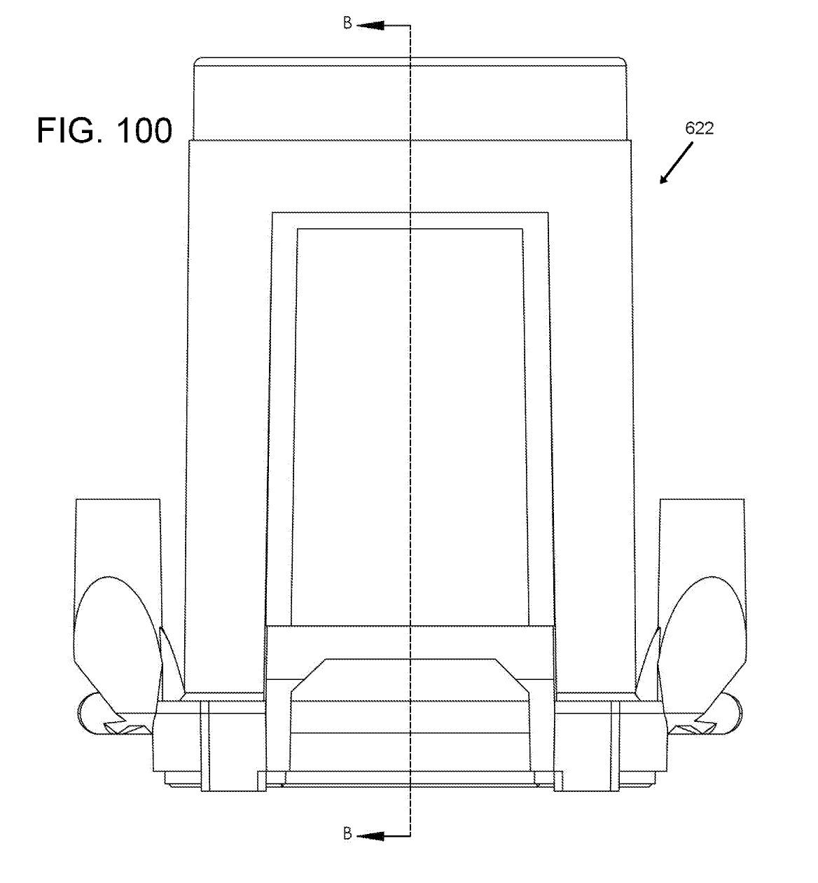

FIG. 100 shows the reservoir retainer 622 of FIG. 94, including a line B-B to show where a cross section is being taken.

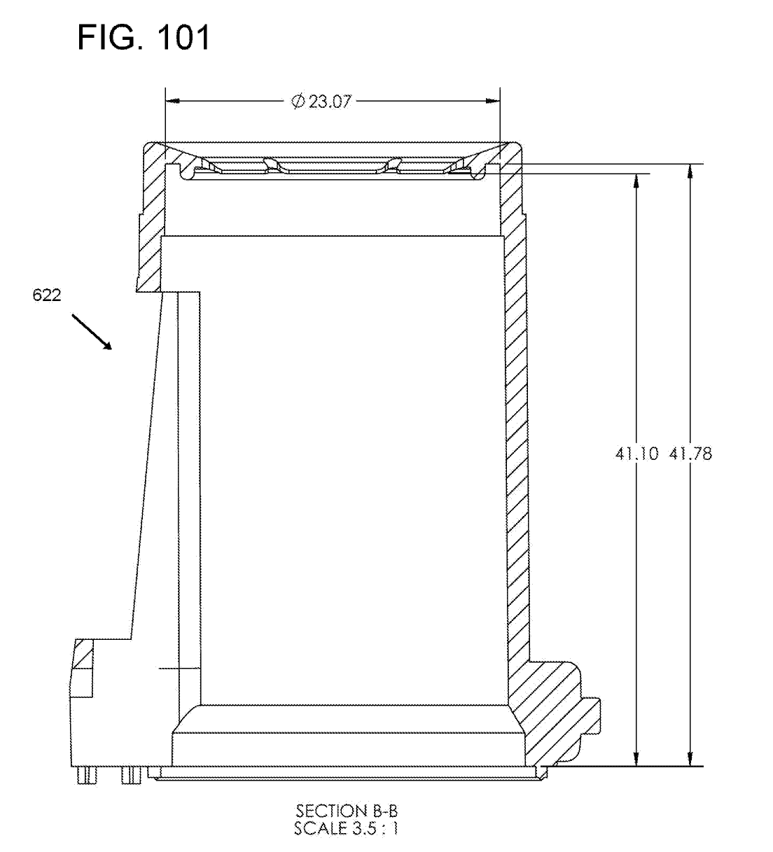

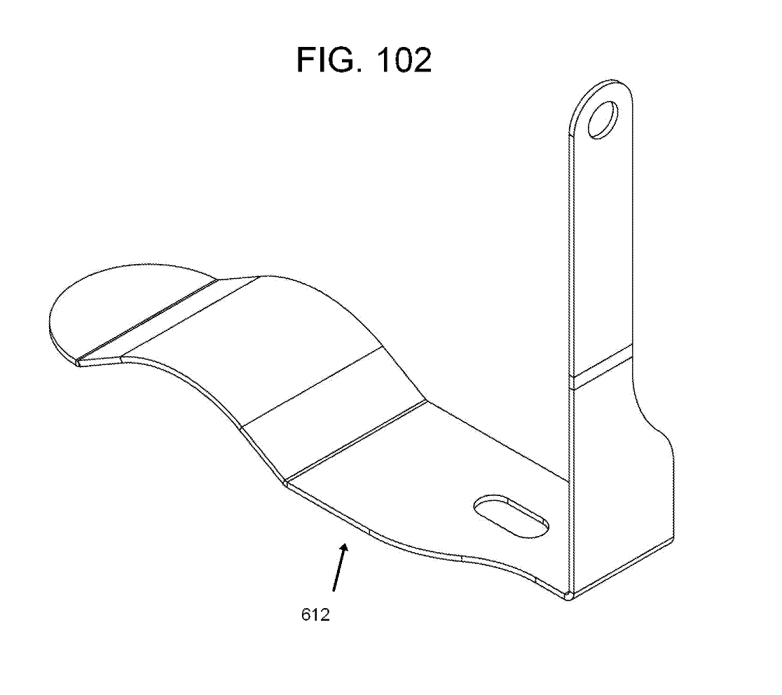

FIG. 101 shows the cross section of the reservoir retainer 622 as shown in FIG. 100.

FIG. 102 shows a perspective view of battery contact C 612.

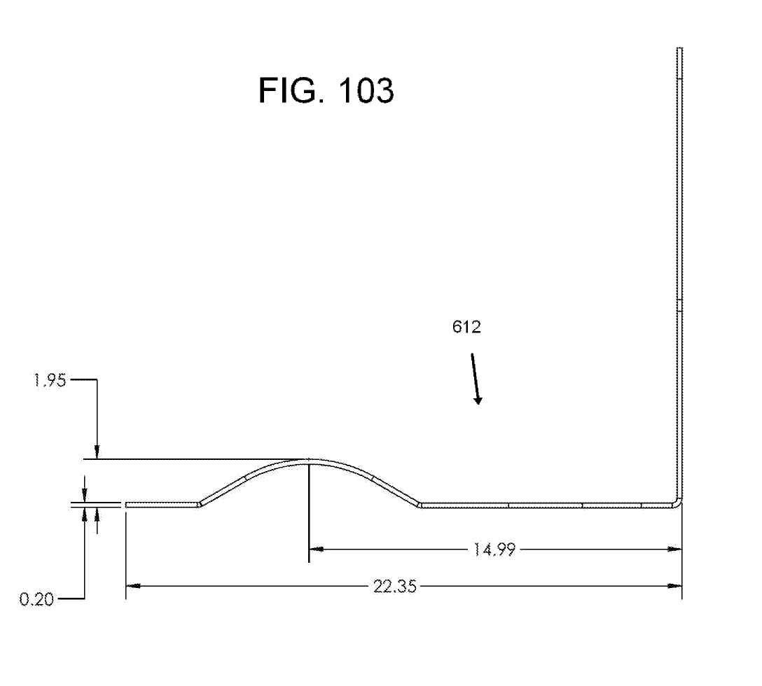

FIG. 103 shows a side view of battery contact 612.

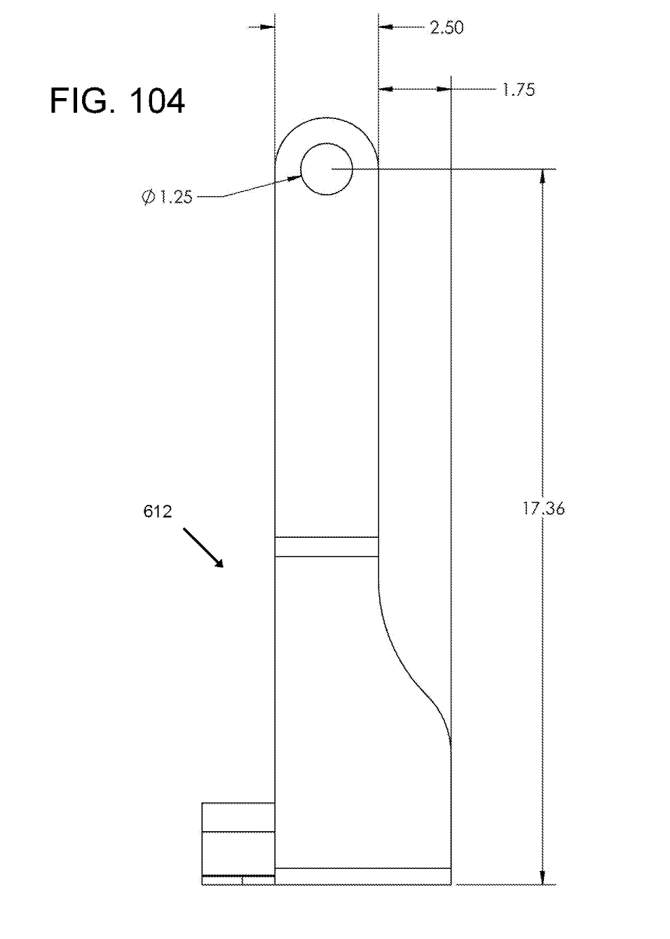

FIG. 104 shows another side view of battery contact 612.

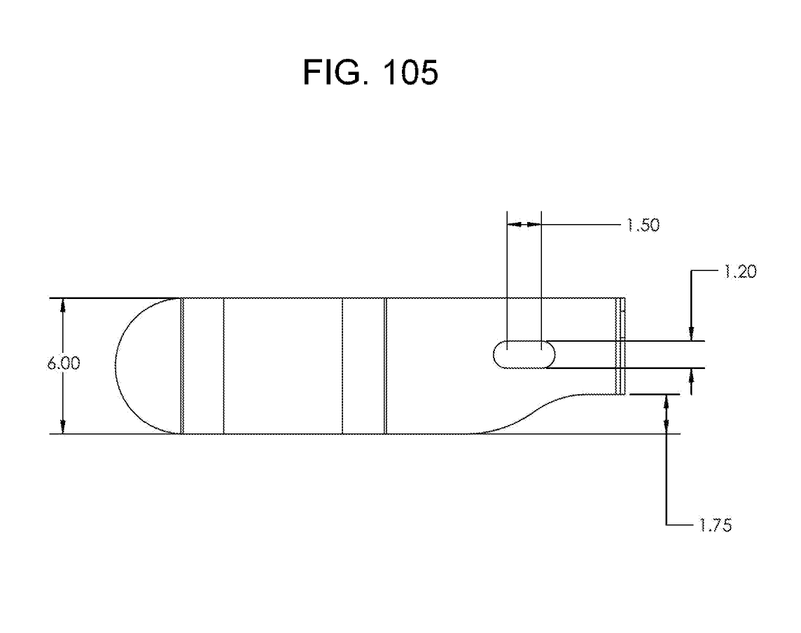

FIG. 105 shows a top view of battery contact 612.

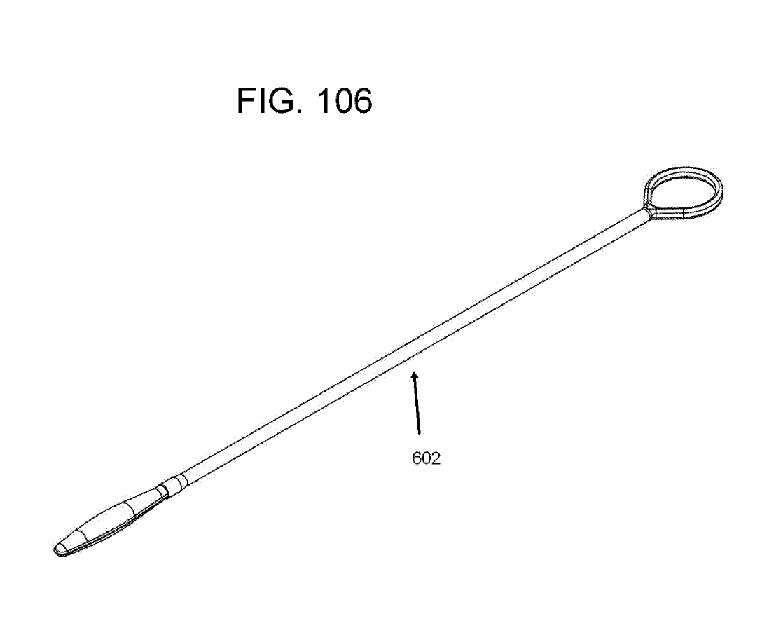

FIG. 106 shows a perspective view of a five millimeter wiping wand 602.

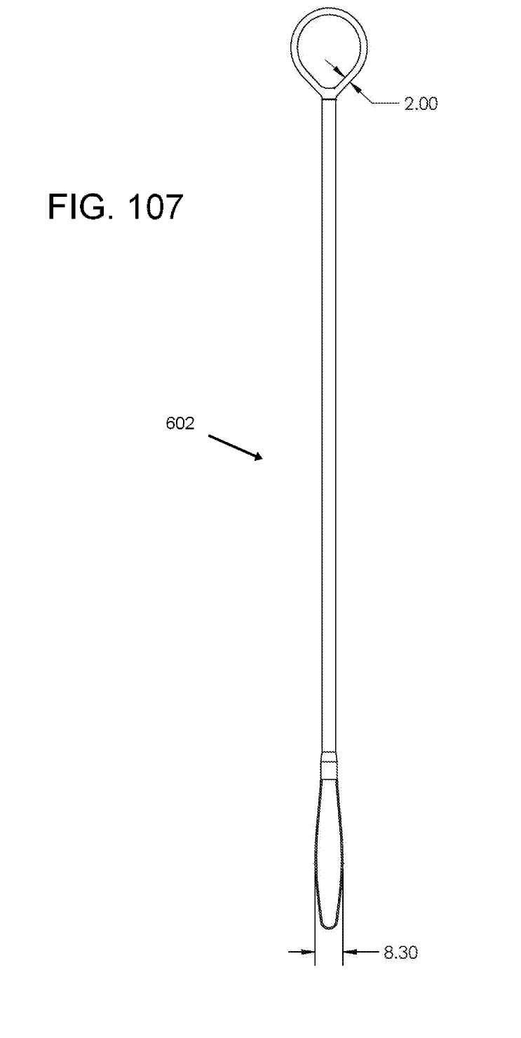

FIG. 107 shows a side view of the five millimeter wiping wand 602.

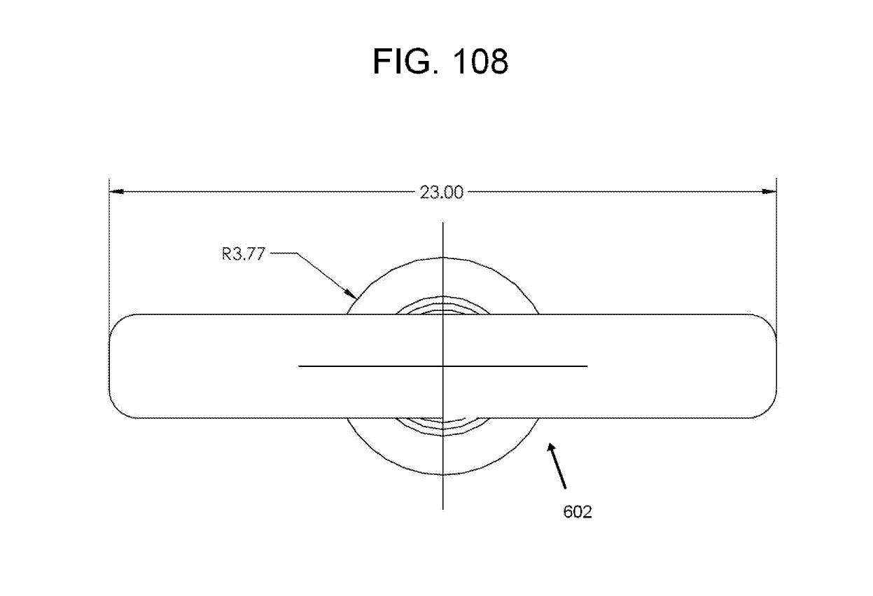

FIG. 108 shows an end view of the five millimeter wiping wand 602.

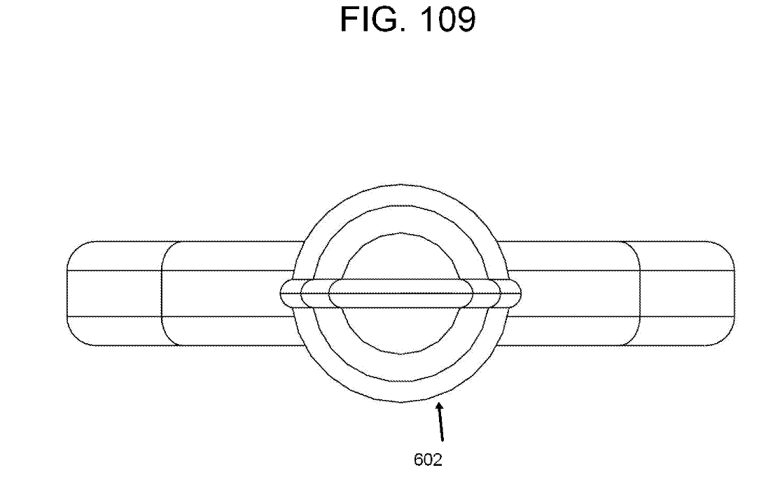

FIG. 109 shows an end view, opposite of the end shown in FIG. 108, of the five millimeter wiping wand 602.

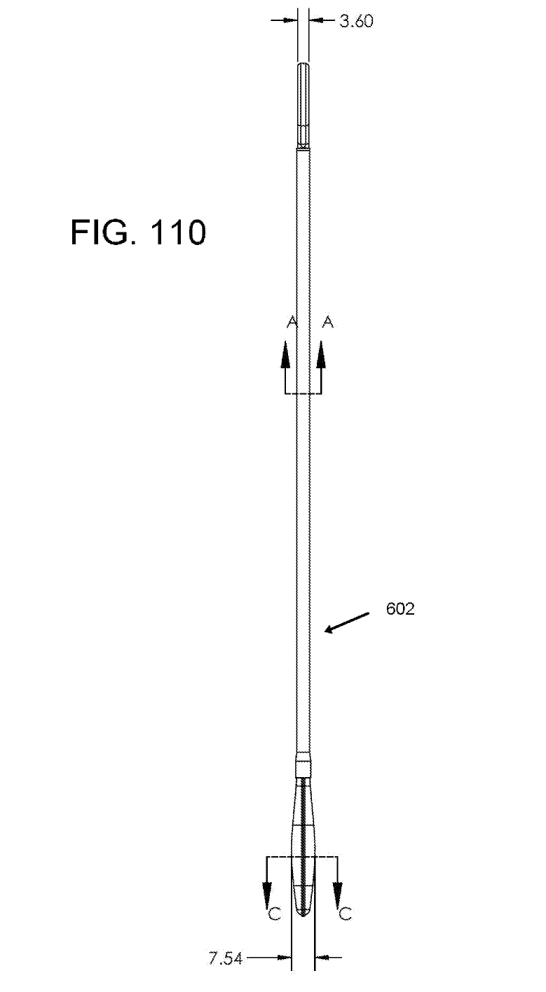

FIG. 110 shows the side view of the five millimeter wiping wand 602 as shown in FIG. 107, with a line A-A to show where a cross section is being taken and a line C-C to show where a different cross section is being taken.

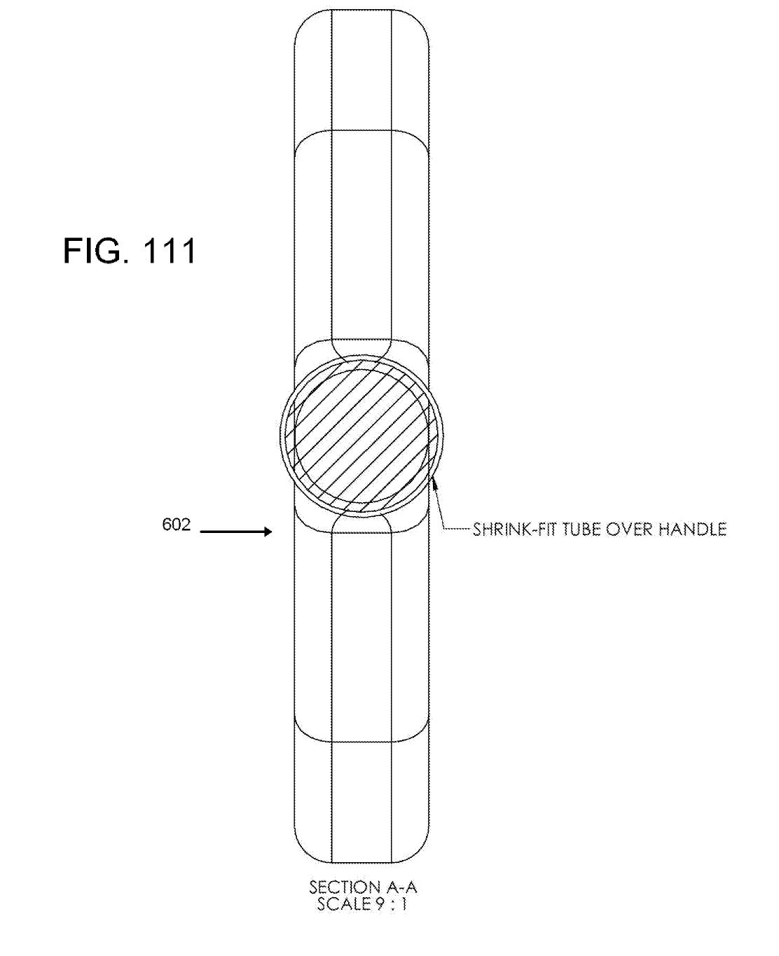

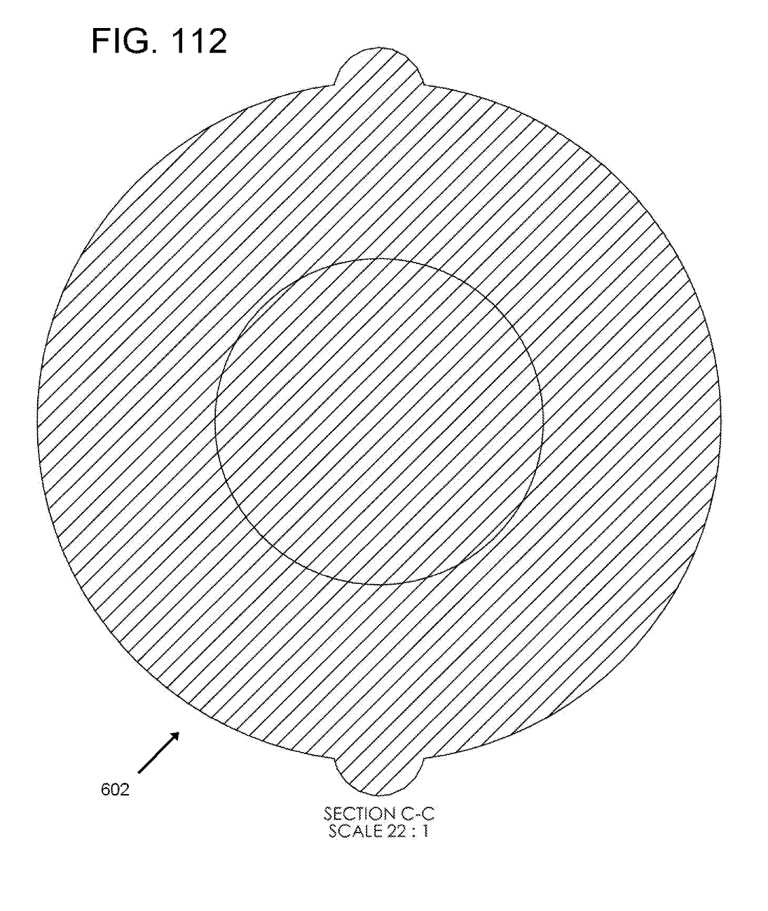

FIG. 111 shows the cross section of the five millimeter wiping wand 602 as shown by the line A-A in FIG. 110.

FIG. 112 shows the cross section of the five millimeter wiping wand 602 as shown by the line C-C in FIG. 110.

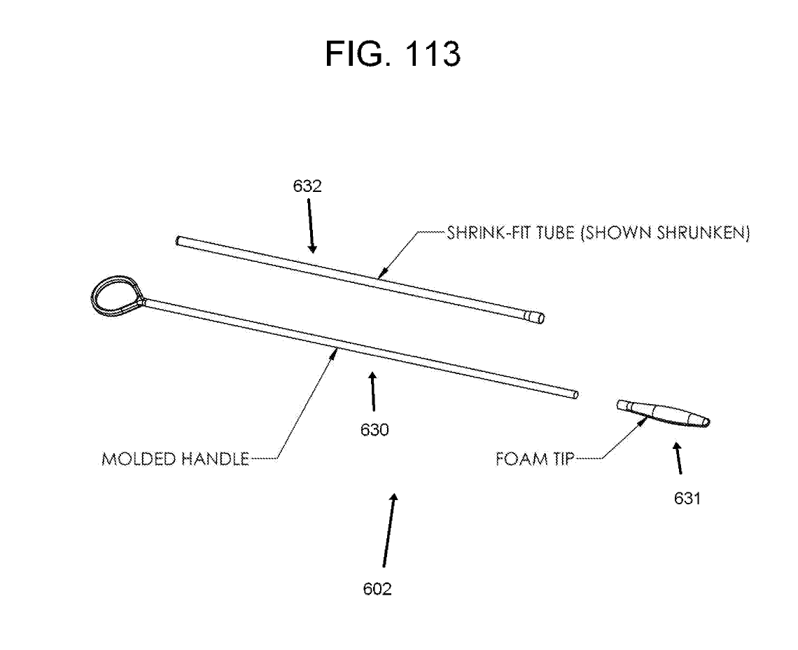

FIG. 113 shows a perspective view of the components of the five millimeter wiping wand 602, including a molded handle 630, a foam tip 631, and a shrink-fit tube 632 (shown shrunken).

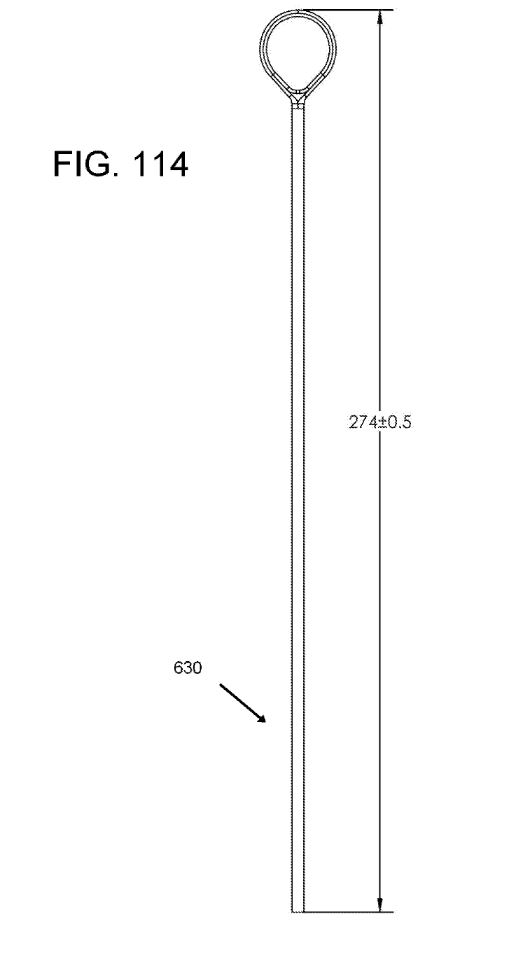

FIG. 114 shows a side view of the molded handle 630.

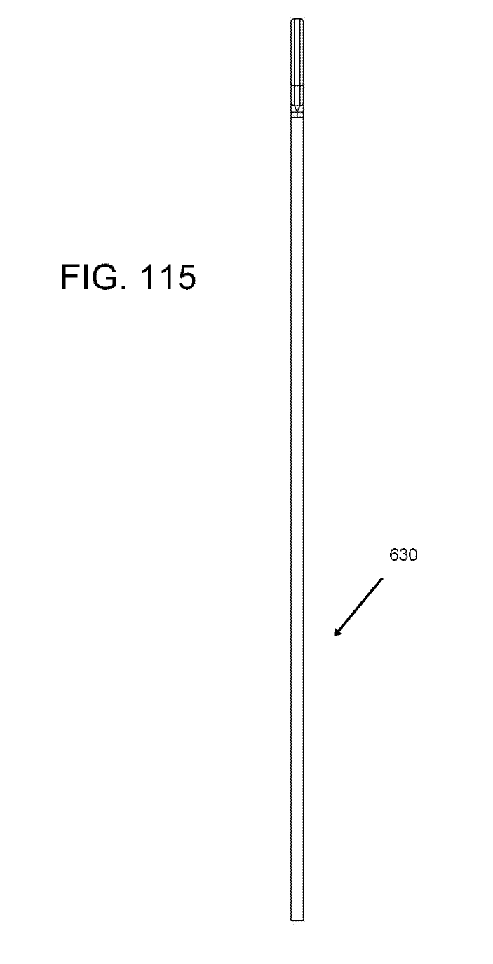

FIG. 115 shows an additional side view of the molded handle 630.

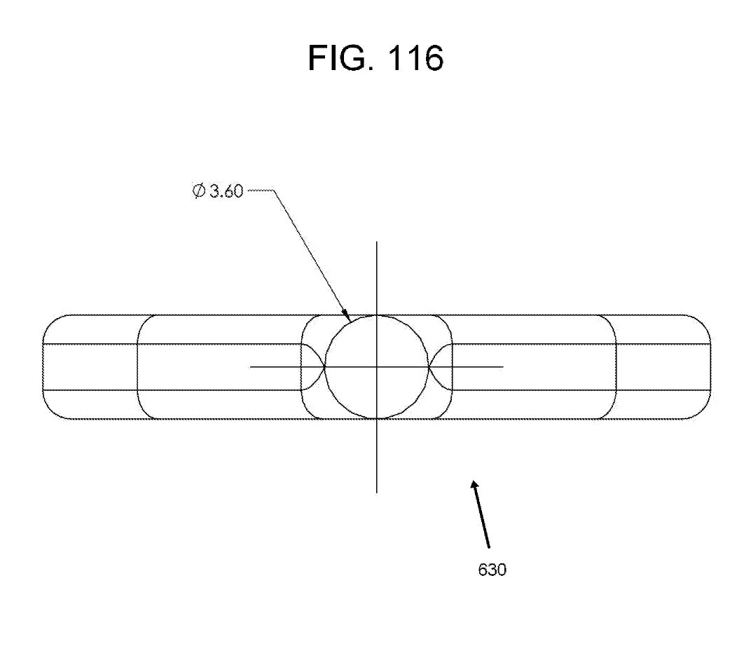

FIG. 116 shows a top view of the molded handle 630.

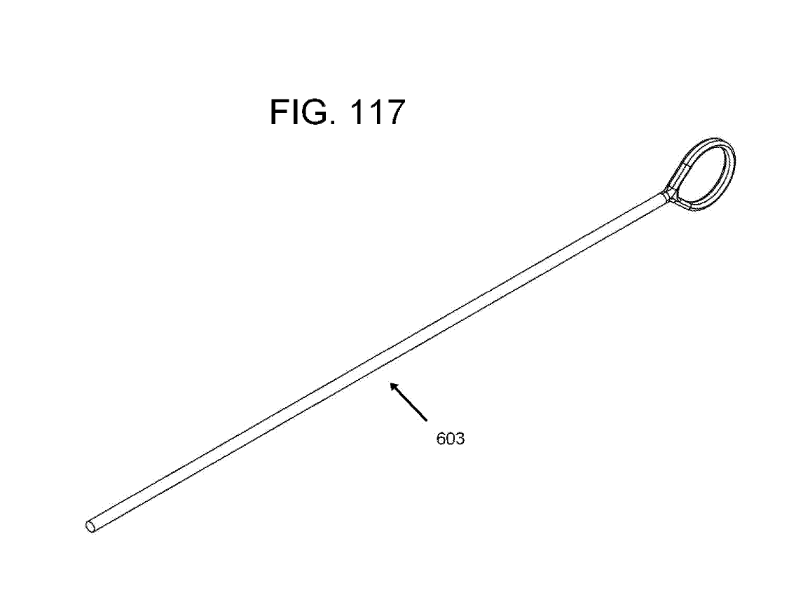

FIG. 117 shows a perspective view of a twelve millimeter wiping wand 603.

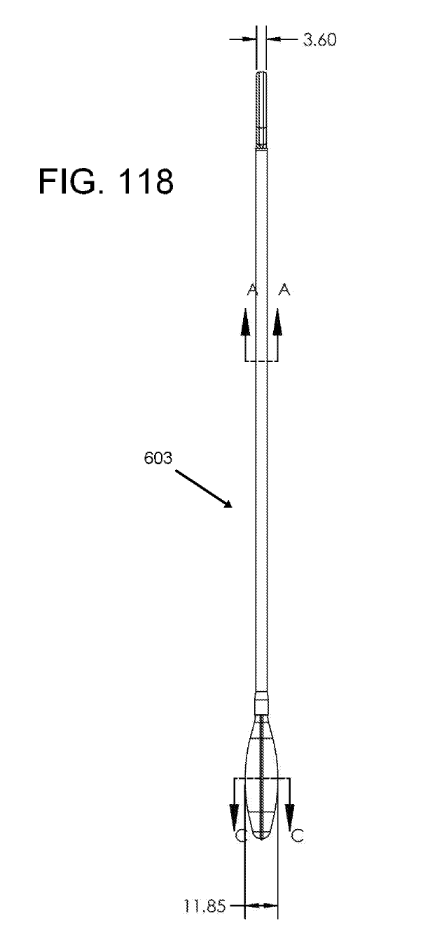

FIG. 118 shows a side view of the twelve millimeter wiping wand 603, with a line A-A to show where a cross section is being taken and a line C-C to show where a different cross section is being taken.

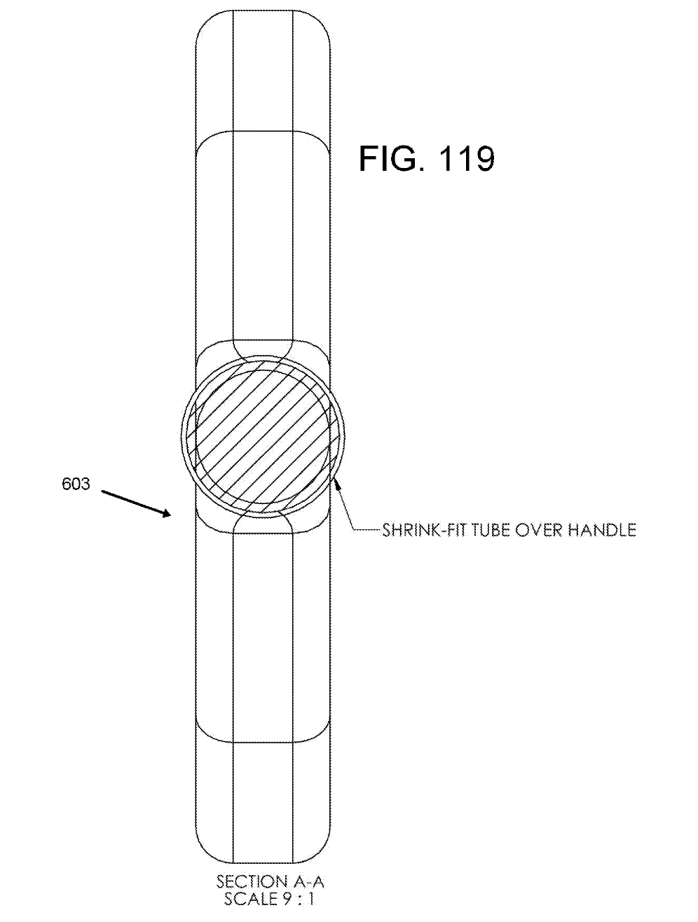

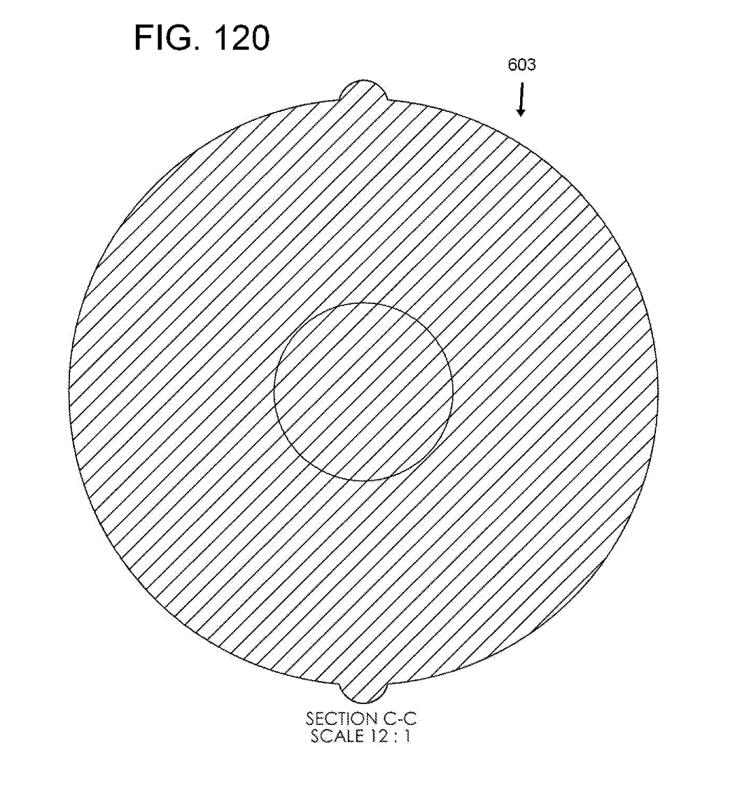

FIG. 119 shows the cross section of the twelve millimeter wiping wand 603 as shown by the line A-A in FIG. 118.

FIG. 120 shows the cross section of the twelve millimeter wiping wand 603 as shown by the line C-C in FIG. 118.

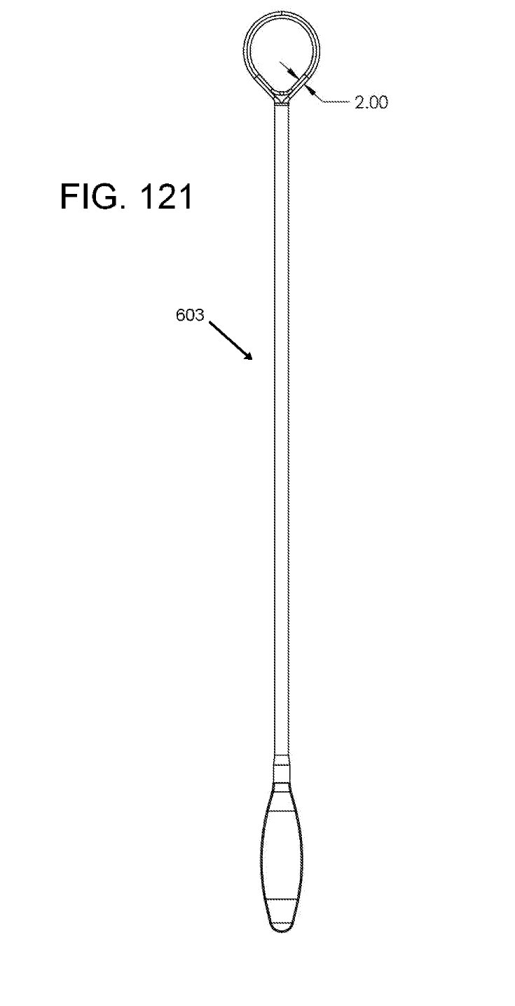

FIG. 121 shows a side view of the twelve millimeter wiping wand 603.

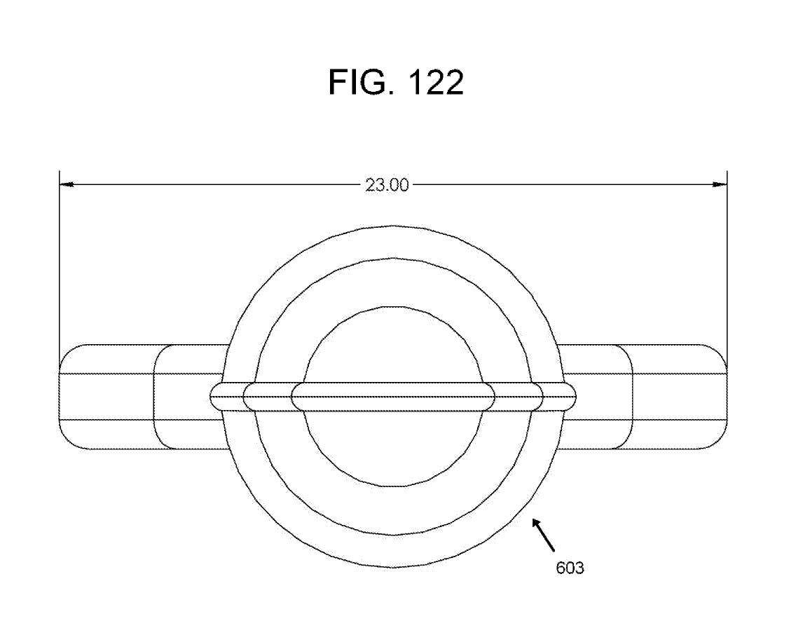

FIG. 122 shows an end view of the twelve millimeter wiping wand 603.

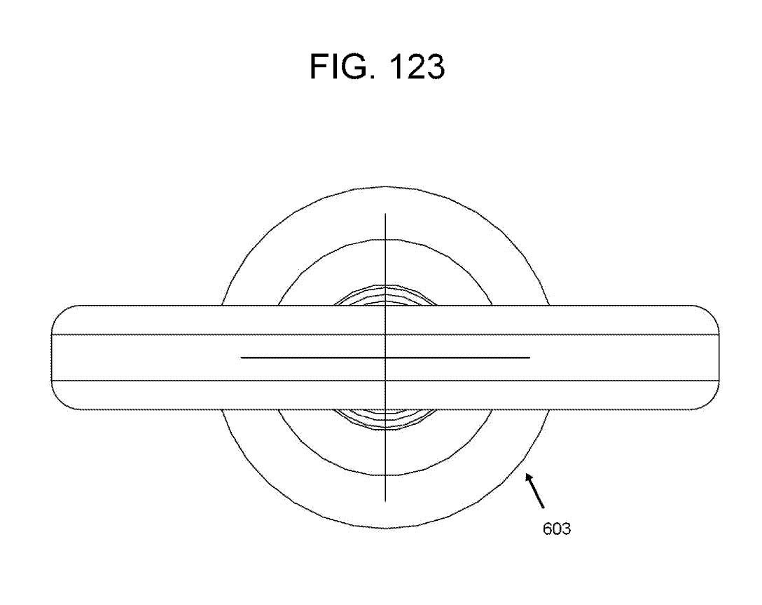

FIG. 123 shows an end view, opposite of the end shown in FIG. 122, of the twelve millimeter wiping wand 603.

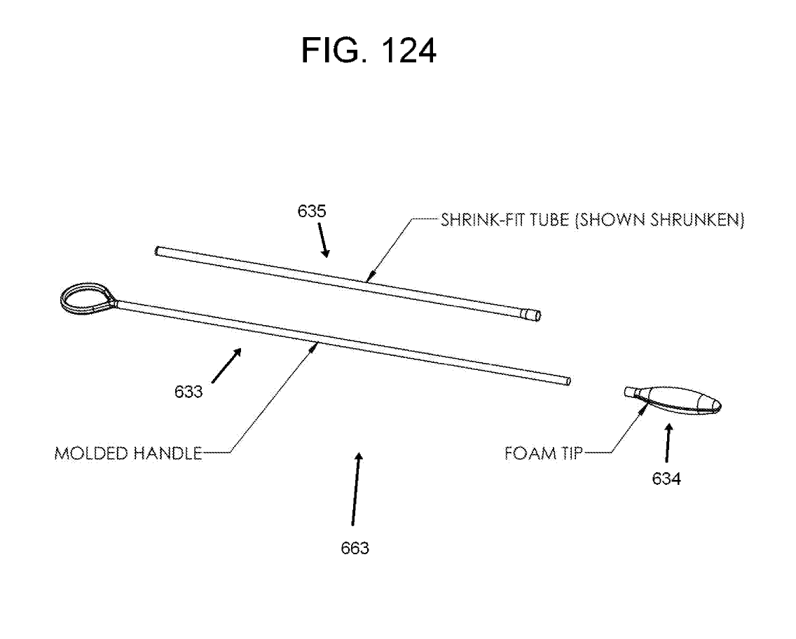

FIG. 124 shows a perspective view of the components of the twelve millimeter wiping wand 603, including a molded handle 633, a foam tip 634, and a shrink-fit tube 635 (shown shrunken).

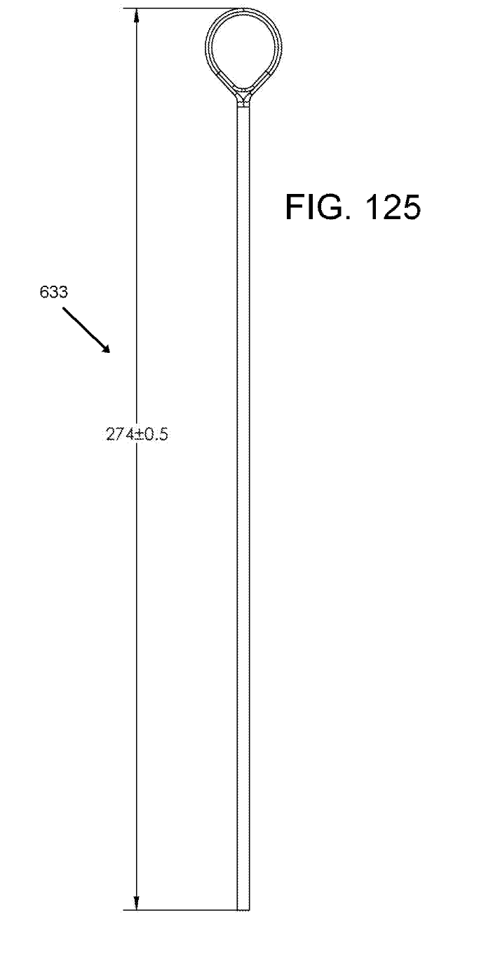

FIG. 125 shows a side view of the molded handle 633.

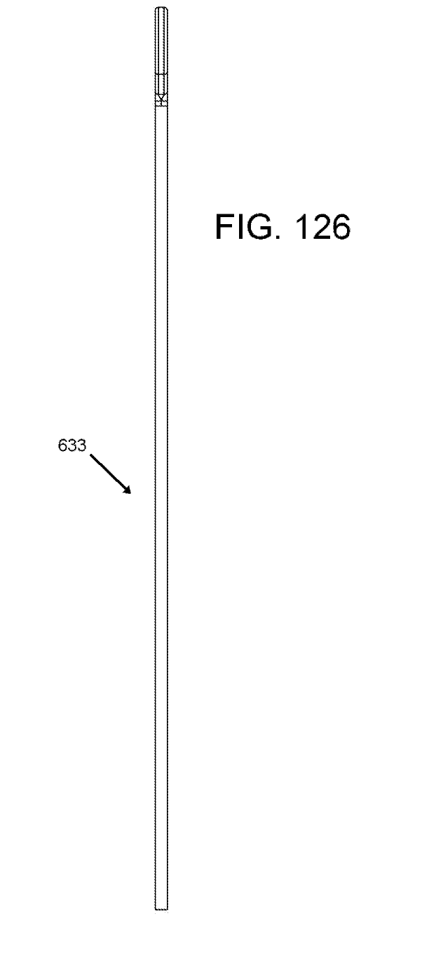

FIG. 126 shows an additional side view of the molded handle 633.

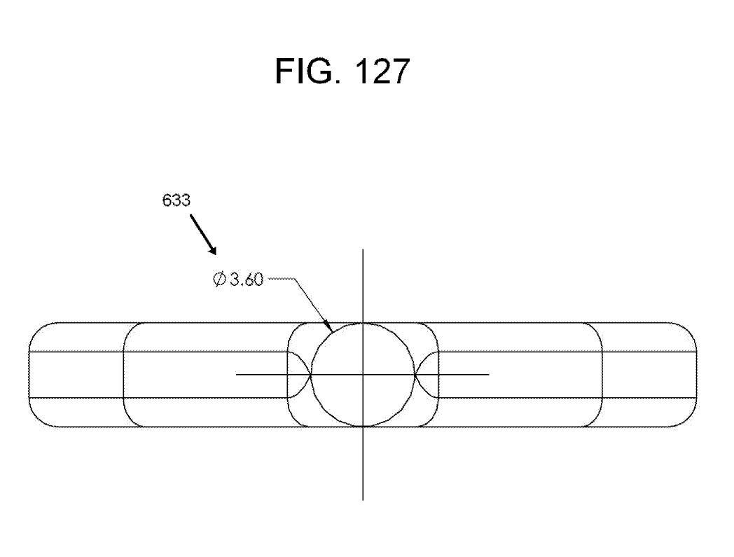

FIG. 127 shows a top view of the molded handle 633.

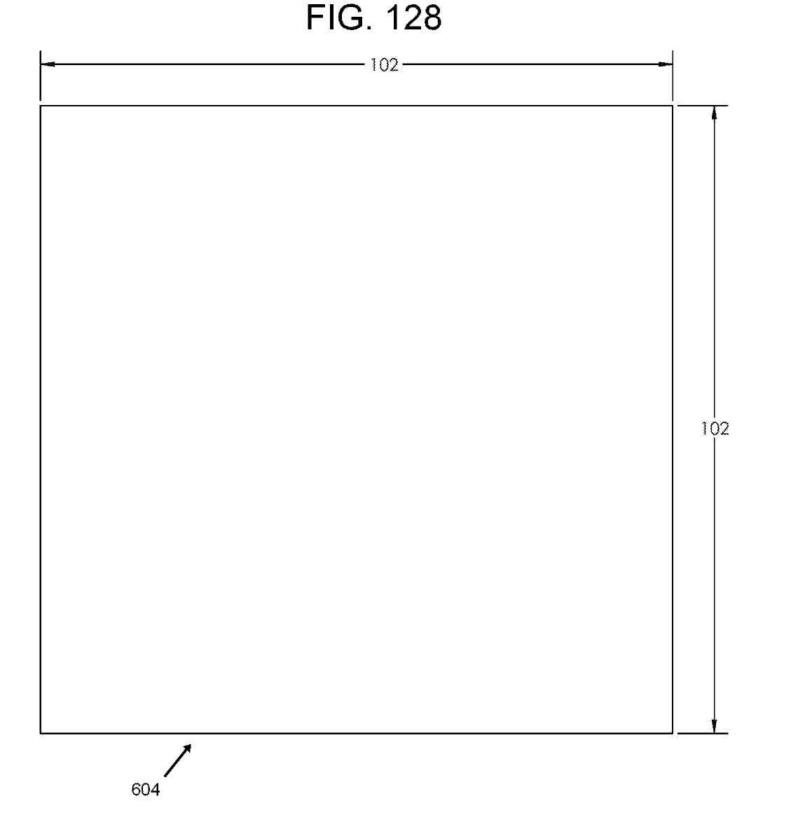

FIG. 128 shows a top view of a microfiber cloth 604. The microfiber cloth 604 has a possible width of 102 millimeters and a possible length of 102 millimeters.

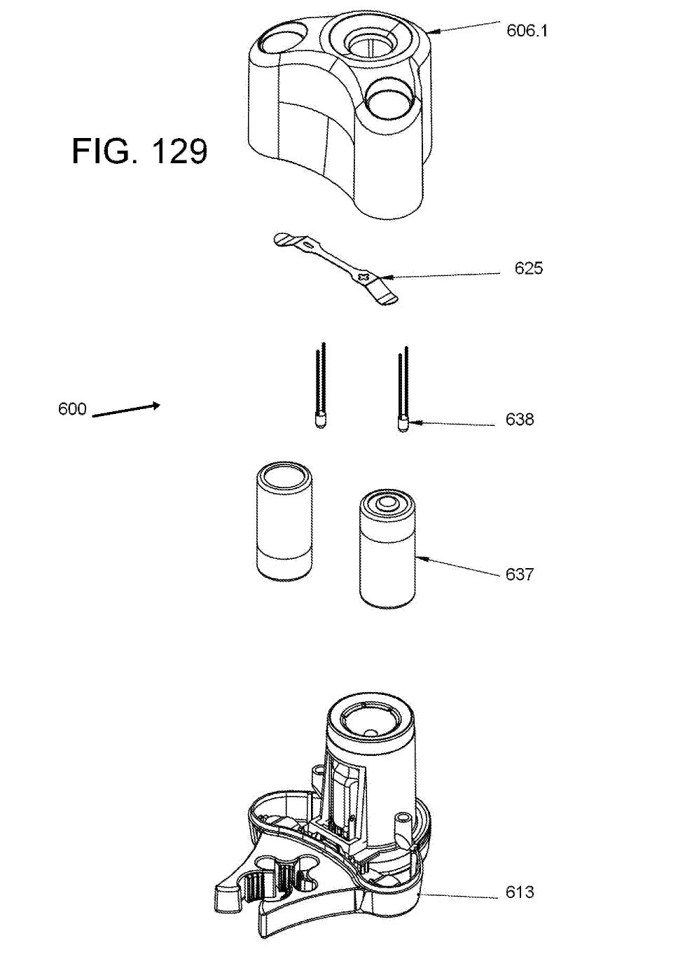

FIG. 129 shows an expanded view of the device 600, including the housing top 606.1, the battery

contact A 625, two 123 lithium batteries 637, two LED TT OVLBG4C7 638, and the assembled bottom assembly 613.

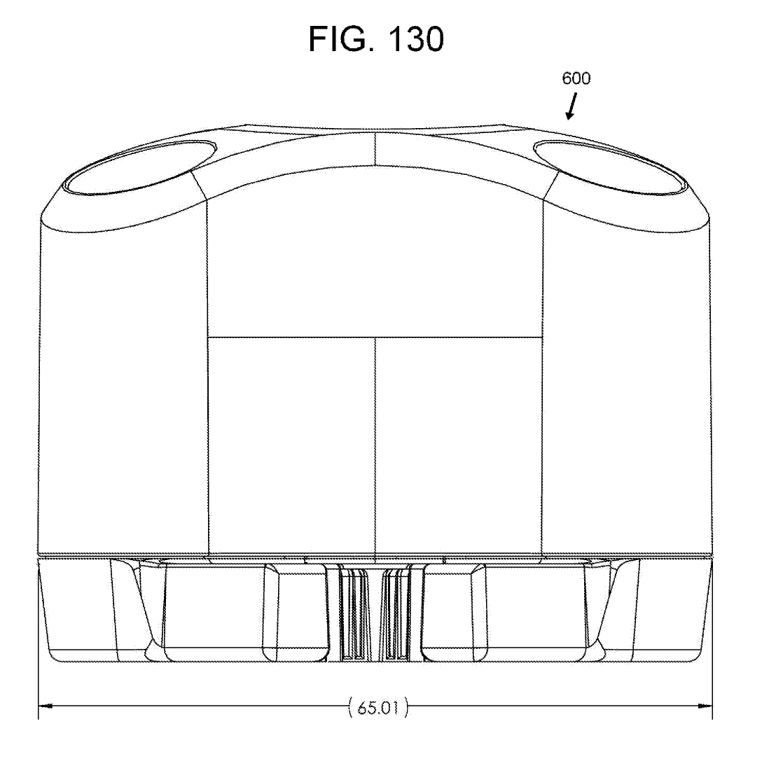

FIG. 130 shows a side view of the completed assembly 600.

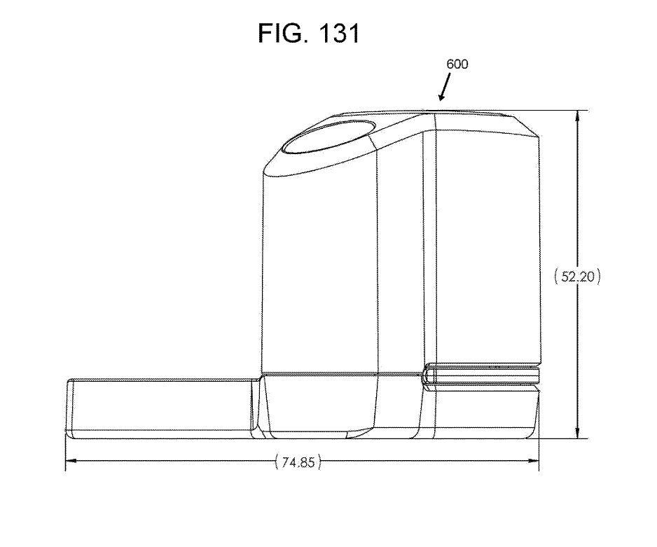

FIG. 131 shows another side view of the completed assembly of the device 600.

An example of a robotic surgery system, such as the Da Vinci Surgical System, is made by Intuitive Surgical, Inc., located at 1266 Kifer Road #101, Sunnyvale, Calif. 94086.

U.S. Pat. No. 8,066,524, having the title “SURGICAL SYSTEM WITH ELECTRO-MECHANICAL INTERFACES TO MOUNT ROBOTIC SURGICAL ARMS,” issued on Nov. 29, 2011, is hereby incorporated by reference as if set forth in its entirety herein except for the exceptions indicated herein.

U.S. patent application Ser. No. 16/110,035, filed on Aug. 23, 2018, is hereby incorporated by reference as if set forth in its entirety herein except for the exceptions indicated herein.

The following patents and patent publications are hereby incorporated by reference: U.S. Patent Application Publication No. 2012/0184897, having the title “INTEGRATED SYSTEMS AND METHODS FOR MAINTENANCE AND MANAGEMENT OF AN INTRAABDOMINAL GAS ENVIRONMENT DURING LAPAROSCOPIC SURGERY,” published Jul. 19, 2012; U.S. Patent Application Publication No. 2012/0197084,

having the title “SYSTEMS AND METHODS FOR OPTIMIZING AND MAINTAINING VISUALIZATION OF A SURGICAL FIELD DURING THE USE OF SURGICAL SCOPES,” published Aug. 2, 2012; U.S. Patent Application Publication No. 2013/0060086, having the title “IMAGING SENSOR PROVIDING IMPROVED VISUALIZATION FOR SURGICAL SCOPES,” published on Mar. 7, 2013; U.S. Patent Application Publication No. 2010/0168520, having the title “VIEW OPTIMIZER AND STABILIZER FOR USE WITH SURGICAL SCOPES,” published Jul. 1, 2010; U.S. Pat. No. 8,535,220, having the title “LAPAROSCOPE CLEANING SYSTEM,” issued Sep. 17, 2013; U.S. Pat. No. 4,392,485, having the title “ENDOSCOPE,” issued on Jul. 12, 1983; U.S. Pat. No. 8,152,717, entitled “DEVICE FOR WHITE BALANCING AND APPLYING AN ANTI-FOG AGENT TO MEDICAL VIDEOSCOPES PRIOR TO MEDICAL PROCEDURES,” issued on Apr. 10, 2012; U.S. Pat. No. 7,803,109, entitled “METHOD AND APPARATUS FOR PROTECTING THE DISTAL LENS OF ENDOSCOPES,” issued on Sep. 28, 2010; U.S. Pat. No. 7,311,660, entitled “METHOD AND APPARATUS FOR HEATING AND APPLYING WARM ANTIFOG SOLUTION TO ENDOSCOPES AS WELL AS A DISTAL LENS PROTECTOR,” issued on Dec. 25, 2007; U.S. Pat. No. 7,080,641, entitled “METHOD AND APPARATUS FOR HEATING STERILE SOLUTIONS DURING MEDICAL PROCEDURES,” issued on Jul. 25, 2006; U.S. Pat. No. 8,870,752, entitled “MEDICAL DEVICE SHEATH,” issued on Oct. 28, 2014; U.S. Pat. No. 8,727,969, entitled “ENDOSCOPE,” issued on May 20, 2014; U.S. Pat. No. 8,696,552, entitled “SELF-CONTAINED STERILIZABLE SURGICAL SYSTEM,” issued on Apr. 15, 2014; U.S. Pat. No. 8,540,745, entitled “BALLOON DISSECTOR WITH CANNULA,” issued on Sep. 24, 2013; U.S. Pat. No. 8,517,918, entitled “OPTICAL TROCAR WITH SCOPE HOLDING ASSEMBLY,” issued on Aug. 27, 2013; U.S. Pat. No. 8,467,589, entitled “HYBRID REGISTRATION METHOD,” issued on Jun. 18, 2013; U.S. Pat. No. 8,454,645, entitled “BALLOON DISSECTOR WITH CANNULA,” issued on Jun. 4, 2013; U.S. Pat. No. 8,452,068, entitled “HYBRID REGISTRATION METHOD,” issued on May 28, 2013; U.S. Pat. No. 7,955,330, entitled “MULTI-PORT SIDE-FIRE COAGULATOR,” issued Jun. 7, 2011; and U.S. Pat. No. 7,927,330, entitled “MULTI-PORT SIDE-FIRE COAGULATOR,” issued Apr. 19, 2011.

Words relating to the opinions and judgments of the author of all patents, patent applications, patent publications, and other documents cited herein and not directly relating to the technical details of the description of the embodiments therein are not incorporated by reference.

The words all, always, absolutely, consistently, preferably, guarantee, particularly, constantly, ensure, necessarily, immediately, endlessly, avoid, exactly, continually, expediently, ideal, need, must, only, perpetual, precise, perfect, require, requisite, simultaneous, total, unavoidable, and unnecessary, or words substantially equivalent to the above-mentioned words in this sentence, when not used to describe technical features of one or more embodiments of the patents, patent applications, patent publications, and other documents, are not considered to be incorporated by reference herein for any of the patents, patent applications, patent publications, and other documents cited herein.

One feature or aspect of an embodiment is believed at the time of the filing of this patent application to possibly reside broadly in a method of minimizing injuries caused by pressure on portions of a body of a patient in the Trendelenburg position on a surgical operating table, and minimizing unwanted movement of a patient in the Trendelenburg position on a surgical operating table by securing a patient to the surgical operating table, and also minimizing contamination in an operating room, by using a single-use Trendelenburg patient support system comprising: a lift sheet configured to lift and position a patient on a surgical operating table, body straps configured to hold a patient down on a surgical operating table, a single-use, viscoelastic Trendelenburg pad comprising a rectangular shape and viscoelastic polyurethane, and securing straps welded to said single-use, viscoelastic Trendelenburg pad, which said securing straps are configured to secure said single-use, viscoelastic Trendelenburg pad to rails of a surgical operating table, said method comprising the steps of: A) placing a longitudinal edge of said single-use, viscoelastic Trendelenburg pad adjacent and in alignment with a longitudinal edge of a surgical operating table having surgical operating table rails; B) placing a latitudinal edge of said single-use, viscoelastic Trendelenburg pad adjacent and in alignment with a latitudinal edge of said surgical operating table, and thus positioning said single-use, viscoelastic Trendelenburg pad in a position on said surgical operating table where the body of a patient will be lying; C) positioning said securing straps, extending from longitudinal edges of said single-use, viscoelastic Trendelenburg pad, to extend down and away from said single-use, viscoelastic Trendelenburg pad; D) attaching said securing straps to said surgical operating table rails; E) laying said lift sheet over said single-use, viscoelastic Trendelenburg pad; F) laying a patient in a supine position on said lift sheet and said single-use, viscoelastic Trendelenburg pad by positioning said patient so that the shoulders of said patient do not extend past edges of said single-use, viscoelastic Trendelenburg pad, and thereby deforming said single-use, viscoelastic Trendelenburg pad, which said single-use, viscoelastic Trendelenburg pad comprises: sufficient thickness and viscosity to sufficiently cushion the body of said patient to minimize and/or prevent bottoming out on said medical procedure table of one or more of the portions of the body of said patient during positioning of said patient and during a surgical procedure, and to minimize injuries from pressure during a surgical procedure performed while a patient is in lithotomy and Trendelenburg positions, and sufficient compliance to conform to a substantial portion of the body of said patient; G) lifting said lift sheet and thereby lifting said patient up and off said single-use, viscoelastic Trendelenburg pad to reposition said patient as or if needed; H) positioning the arms of said patient as or if needed; I) attaching said body straps around said patient and said surgical operating table; J) positioning the legs of said patient in a lithotomy position; K) adjusting the angle of inclination of said surgical operating table to orient said patient at an angle in a Trendelenburg position in which the head of said patient is disposed below the body of said patient, or in which the head of said patient is disposed above the body of said patient, or in which the right side of a patient is disposed above the left side or vice versa, or a combination of any of these positions; and L) assisting in substantially holding the body of said patient on said surgical operating table in said lithotomy and Trendelenburg positions using said single-use, viscoelastic Trendelenburg pad, which said single-use, viscoelastic Trendelenburg pad comprises: sufficient thinness to stabilize said patient on said surgical operating table upon said patient being in said lithotomy and Trendelenburg positions, and sufficient thickness and sufficient compliance to permit formation of a cavity in said single-use, viscoelastic Trendelenburg pad of a depth sufficient to assist in holding said patient on said surgical operating table and/or minimizing undesired movement of the body of said patient on said surgical operating table during a surgical procedure performed while said patient is in said lithotomy and Trendelenburg positions.

Another feature or aspect of an embodiment is believed at the time of the filing of this patent application to possibly reside broadly in the method, wherein: said single-use Trendelenburg patient support system comprises a kit; said step D) comprises using securing straps comprising a central portion, attached to a lower side of said single-use, viscoelastic Trendelenburg pad, and fastener ends, each comprising a hook and loop fastener; said single-use, viscoelastic Trendelenburg pad has a thickness in the range of from three-fourths of an inch to three inches or greater; and said single-use, viscoelastic Trendelenburg pad comprises a viscoelastic polyurethane foam having: a ball rebound sufficiently small to minimize rebound of said patient during an operation; a compression set sufficiently small to minimize discomfort of and injury to said patient on said surgical operating table; an air flow sufficient to provide substantial air flow about said patient to minimize injury to said patient and maintain a patient in a useable Trendelenburg position; an indentation force deflection sufficient to provide a securing hold on said patient; a tensile strength sufficient to minimize tearing of said single-use, viscoelastic Trendelenburg pad; a coefficient of static friction sufficient to assist in minimizing movement of said single-use, viscoelastic Trendelenburg pad on said surgical operating table when said patient is on said surgical operating table; and a density configured to provide said ball rebound, said compression set, said air flow, said indentation force deflection, said tensile strength, and said coefficient of static friction.

Yet another feature or aspect of an embodiment is believed at the time of the filing of this patent application to possibly reside broadly in the method, wherein: said single-use, viscoelastic Trendelenburg pad has a thickness of approximately one inch; said ball rebound is in the range of approximately 0.1 percent to approximately 1.9 percent; said compression set, for a 25 percent compression, is less than 0.3 percent; said air flow is in the range of 0.3 to 1.0 cubic foot per minute; said indentation force deflection is in the range of approximately 10 to approximately 15 pounds; said tensile strength is in the range of approximately 8 pounds per square inch to approximately 12 pounds per square inch; said coefficient of static friction is in the range of 0.2 to 1.0; and said density is in the range of approximately 83 kilograms per cubic meter to approximately 103 kilograms per cubic meter.

Still another feature or aspect of an embodiment is believed at the time of the filing of this patent application to possibly reside broadly in a single-use Trendelenburg patient support system for performing the method, said single-use Trendelenburg patient support system comprising: a lift sheet configured to lift and position a patient on a surgical operating table; body straps configured to hold a patient down on a surgical operating table; a single-use, viscoelastic Trendelenburg pad comprising a rectangular shape and viscoelastic polyurethane; securing straps welded to said single-use, viscoelastic Trendelenburg pad; said securing straps being configured to secure said single-use, viscoelastic Trendelenburg pad to rails of a surgical operating table; said single-use, viscoelastic Trendelenburg pad comprising: sufficient thickness and viscosity to sufficiently cushion the body of a patient to minimize and/or prevent bottoming out on said medical procedure table of one or more of the portions of the body of a patient during positioning of a patient and during a surgical procedure, and to minimize injuries from pressure during a surgical procedure; sufficient compliance to conform to a substantial portion of the body of a patient; sufficient thinness to stabilize a patient on said surgical operating table upon a patient being in lithotomy and Trendelenburg positions; and sufficient thickness and sufficient compliance to permit formation of a cavity in said single-use, viscoelastic Trendelenburg pad of a depth sufficient to assist in holding a patient on said surgical operating table and/or minimizing undesired movement of the body of a patient on said surgical operating table during a surgical procedure performed while a patient is in lithotomy and Trendelenburg positions.

A further feature or aspect of an embodiment is believed at the time of the filing of this patent application to possibly reside broadly in the single-use Trendelenburg patient support system, wherein: said single-use Trendelenburg patient support system comprises a kit; said body straps comprise hook and loop fasteners; said securing straps each comprise a central portion, attached to a lower side of said single-use, viscoelastic Trendelenburg pad, and fastener ends, each comprising a hook and loop fastener; said single-use, viscoelastic Trendelenburg pad has a thickness of approximately one inch; and said single-use, viscoelastic Trendelenburg pad comprises a viscoelastic polyurethane having: a ball rebound in the range of approximately 0.1 percent to approximately 1.9 percent; a compression set, for a 25 percent compression, of less than 0.3 percent; an air flow in the range of 0.3 to 1.0 cubic foot per minute; an indentation force deflection in the range of approximately 10 to approximately 15 pounds; a tensile strength in the range of approximately 8 pounds per square inch to approximately 12 pounds per square inch; a coefficient of static friction in the range of 0.2 to 1.0; and a density in the range of approximately 83 kilograms per cubic meter to approximately 103 kilograms per cubic meter.

Another feature or aspect of an embodiment is believed at the time of the filing of this patent application to possibly reside broadly in a method of minimizing injuries caused by pressure on portions of a body of a patient and minimizing unwanted movement of a patient upon securing a patient to a medical procedure table, such as a surgical operating table or a patient examination table, and upon placing said medical procedure table in an inclined position, using a patient support system comprising a viscoelastic pad and securing straps, said method comprising the steps of: A) positioning said viscoelastic pad in a position on said medical procedure table where the body of a patient will be lying; B) attaching said securing straps, connected to said viscoelastic pad, to said medical procedure table; C) positioning a patient on said viscoelastic pad and thereby deforming said viscoelastic pad, which said viscoelastic pad comprises: sufficient thickness and viscosity to sufficiently cushion the body of said patient to minimize and/or prevent bottoming out on said medical procedure table of one or more of the portions of the body of said patient during positioning of said patient and during a medical procedure, and to minimize injuries from pressure during a medical procedure, and sufficient compliance to conform to a substantial portion of the body of said patient; D) adjusting the angle of inclination of said medical procedure table to orient said patient at an angle in an inclined position, in which the head of said patient is disposed below the body of said patient, or in which the head of said patient is disposed above the body of said patient, or in which the right side of a patient is disposed above the left side or vice versa, or a combination of any of these positions; and E) assisting in substantially holding the body of said patient on said medical procedure table using said viscoelastic pad, of which said viscoelastic pad comprises: sufficient thinness to stabilize said patient on said medical procedure table upon said patient being in said inclined position, and sufficient thickness and sufficient compliance to permit formation of a cavity in said viscoelastic pad of a depth sufficient to assist in holding a patient on a medical procedure table and/or minimizing undesired movement of the body of a patient on a medical procedure table during a medical procedure performed while a patient is in an inclined position.

Yet another feature or aspect of an embodiment is believed at the time of the filing of this patent application to possibly reside broadly in the method, wherein: said patient support system further comprises at least one body strap; said patient support system comprises a kit; said method further comprises attaching said at least one body strap around said patient and said medical procedure table between steps C) and D); said step B) comprises using securing straps comprising a central portion, attached to a lower side of said viscoelastic pad, and fastener ends, each comprising a hook and loop fastener; said step D) comprises adjusting the angle of inclination of said medical procedure table to orient said patient in a Trendelenburg position; said viscoelastic pad has a thickness in the range of from three-fourths of an inch to three inches or greater; and said viscoelastic pad comprises a viscoelastic polyurethane foam having: a ball rebound sufficiently small to minimize rebound of said patient during an operation; a compression set sufficiently small to minimize discomfort of and injury to said patient on said medical procedure table; an air flow sufficient to provide substantial air flow about said patient to minimize injury to said patient and maintain a patient in an inclined position; an indentation force deflection sufficient to provide a securing hold on said patient; a tensile strength sufficient to minimize tearing of said viscoelastic pad; a coefficient of static friction sufficient to assist in minimizing movement of said viscoelastic pad on said medical procedure table when said patient is on said medical procedure table; and a density configured to provide said ball rebound, said compression set, said air flow, said indentation force deflection, said tensile strength, and said coefficient of static friction.

Still another feature or aspect of an embodiment is believed at the time of the filing of this patent application to possibly reside broadly in a patient support system for performing the method, said patient support system comprising: a viscoelastic pad; securing straps connected to said viscoelastic pad; said securing straps being configured to secure said viscoelastic pad to a medical procedure table; said viscoelastic pad comprising: sufficient thickness and viscosity to sufficiently cushion the body of a patient to minimize and/or prevent bottoming out on a medical procedure table of one or more of the portions of the body of a patient during positioning of a patient and during a medical procedure, and to minimize injuries from pressure during a medical procedure; sufficient compliance to conform to a substantial portion of the body of a patient; sufficient thinness to stabilize a patient on a medical procedure table upon a patient being in an inclined position; sufficient thinness to stabilize a patient on a medical procedure table upon a patient being in an inclined position, and sufficient thickness and sufficient compliance to permit formation of a cavity in said viscoelastic pad of a depth sufficient to assist in holding a patient on a medical procedure table and/or minimizing undesired movement of the body of a patient on a medical procedure table during a medical procedure performed while a patient is in an inclined position.

A further feature or aspect of an embodiment is believed at the time of the filing of this patent application to possibly reside broadly in the patient support system, wherein: said patient support system further comprises at least one body strap configured to hold a patient down on a medical procedure table while a patient is in a Trendelenburg position; said patient support system comprises a kit; said at least one body strap comprises hook and loop fasteners; said securing straps each comprise a central portion, attached to a lower side of said viscoelastic pad, and fastener ends, each comprising a hook and loop fastener; said viscoelastic pad has a thickness in the range of from three-fourths of an inch to three inches or greater; and said viscoelastic pad comprises a viscoelastic polyurethane foam having: a ball rebound in the range of approximately 0.1 percent to approximately 5 percent; a compression set, for a 25 percent compression, of less than one percent; a air flow in the range of 0.1 to 3.0 cubic foot per minute; a indentation force deflection in the range of approximately 7 to approximately 18 pounds; a tensile strength in the range of approximately 5 pounds per square inch to approximately 15 pounds per square inch; a coefficient of static friction is at least 0.2; and a density in the range of approximately 75 kilograms per cubic meter to approximately 110 kilograms per cubic meter.

Another feature or aspect of an embodiment is believed at the time of the filing of this patent application to possibly reside broadly in a patient support arrangement configured to support and assist in holding a patient on an inclined medical procedure table, said patient support arrangement comprising: a pad comprising a thickness and a viscosity sufficient to cushion the body of a patient to minimize injuries from pressure during a medical procedure, and to minimize and/or prevent bottoming out on a medical procedure table of one or more of the portions of the body of a patient both during positioning of a patient and during a medical procedure; said pad comprising a compliance sufficient to permit said pad to conform to a substantial portion of the body of a patient; said thickness being sufficiently small to stabilize a patient on a medical procedure table upon a patient being in an inclined position; and said thickness and said compliance being sufficient to permit deformation of said pad to a depth sufficient to assist in holding a patient on a medical procedure table, and to assist in minimizing undesired movement of the body of a patient on a medical procedure table, during a medical procedure performed while a patient is in an inclined position.

Yet another feature or aspect of an embodiment is believed at the time of the filing of this patent application to possibly reside broadly in the patient support arrangement, wherein said pad comprises a rebound, such as ball rebound, being sufficiently small to minimize undesirable movement of a patient during a medical procedure.

Still another feature or aspect of an embodiment is believed at the time of the filing of this patent application to possibly reside broadly in the patient support arrangement, wherein said ball rebound is in the range of approximately 0.1 percent to approximately 5 percent.

A further feature or aspect of an embodiment is believed at the time of the filing of this patent application to possibly reside broadly in the patient support arrangement, wherein said ball rebound is in the range of approximately 0.1 percent to approximately 1.9 percent.

One feature or aspect of an embodiment is believed at the time of the filing of this patent application to possibly reside broadly in the patient support arrangement, wherein said pad comprises a compression set being sufficiently small to minimize discomfort of and injury to a patient on a medical procedure table.

Another feature or aspect of an embodiment is believed at the time of the filing of this patent application to possibly reside broadly in the patient support arrangement, wherein said compression set, for a 25 percent compression, is less than one percent.

Yet another feature or aspect of an embodiment is believed at the time of the filing of this patent application to possibly reside broadly in the patient support arrangement, wherein said compression set, for a 25 percent compression, is less than 0.3 percent.

Still another feature or aspect of an embodiment is believed at the time of the filing of this patent application to possibly reside broadly in the patient support arrangement, wherein said pad comprises an indentation force deflection sufficient to provide a securing hold on a patient.

A further feature or aspect of an embodiment is believed at the time of the filing of this patent application to possibly reside broadly in the patient support arrangement, wherein said indentation force deflection is in the range of approximately 7 to approximately 18 pounds.

Another feature or aspect of an embodiment is believed at the time of the filing of this patent application to possibly reside broadly in the patient support arrangement, wherein said indentation force deflection is in the range of approximately 10 to approximately 15 pounds.

Yet another feature or aspect of an embodiment is believed at the time of the filing of this patent application to possibly reside broadly in the patient support arrangement, wherein said pad comprises: a compression set being sufficiently small to minimize discomfort of and injury to a patient on a medical procedure table; and an indentation force deflection sufficient to provide a securing hold on a patient.

Still another feature or aspect of an embodiment is believed at the time of the filing of this patent application to possibly reside broadly in the patient support arrangement, wherein: said pad has a thickness in the range of from three-fourths of an inch to three inches or greater; and said pad comprises: an air flow sufficient to provide substantial air flow about a patient to minimize injury to a patient and maintain a patient in an inclined position; a tensile strength sufficient to minimize tearing of said pad; a coefficient of static friction sufficient to assist in minimizing movement of said pad on a medical procedure table when a patient is on a medical procedure table; and a density configured to provide said ball rebound, said compression set, said air flow, said indentation force deflection, said tensile strength, and said coefficient of static friction.

A further feature or aspect of an embodiment is believed at the time of the filing of this patent application to possibly reside broadly in the patient support arrangement, wherein: said ball rebound is in the range of approximately 0.1 percent to approximately 5 percent; said compression set, for a 25 percent compression, is less than one percent; said air flow is in the range of 0.1 to 3.0 cubic foot per minute; said indentation force deflection is in the range of approximately 7 to approximately 18 pounds; said tensile strength is in the range of approximately 5 pounds per square inch to approximately 15 pounds per square inch; said coefficient of static friction is at least 0.2; and said density is in the range of approximately 75 kilograms per cubic meter to approximately 110 kilograms per cubic meter.

Another feature or aspect of an embodiment is believed at the time of the filing of this patent application to possibly reside broadly in the patient support arrangement, wherein: said ball rebound is in the range of approximately 0.1 percent to approximately 1.9 percent; said compression set, for a 25 percent compression, is less than 0.3 percent; said air flow is in the range of 0.3 to 1.0 cubic foot per minute; said indentation force deflection is in the range of approximately 10 to approximately 15 pounds; said tensile strength is in the range of approximately 8 pounds per square inch to approximately 12 pounds per square inch; said coefficient of static friction is in the range of 0.2 to 1.0; and said density is in the range of approximately 83 kilograms per cubic meter to approximately 103 kilograms per cubic meter.

Yet another feature or aspect of an embodiment is believed at the time of the filing of this patent application to possibly reside broadly in the patient support arrangement, wherein at least one of a)-g): a) said ball rebound is in the range of approximately 0.1 percent to approximately 5 percent; b) said compression set, for a 25 percent compression, is less than one percent; c) said air flow is in the range of 0.1 to 3.0 cubic foot per minute; d) said indentation force deflection is in the range of approximately 7 to approximately 18 pounds; e) said tensile strength is in the range of approximately 5 pounds per square inch to approximately 15 pounds per square inch; f) said coefficient of static friction is at least 0.2; and g) said density is in the range of approximately 75 kilograms per cubic meter to approximately 110 kilograms per cubic meter.

Still another feature or aspect of an embodiment is believed at the time of the filing of this patent application to possibly reside broadly in the patient support arrangement, wherein at least one of h)-n): h) said ball rebound is in the range of approximately 0.1 percent to approximately 1.9 percent; i) said compression set, for a 25 percent compression, is less than 0.3 percent; j) said air flow is in the range of 0.3 to 1.0 cubic foot per minute; k) said indentation force deflection is in the range of approximately 10 to approximately 15 pounds; l) said tensile strength is in the range of approximately 8 pounds per square inch to approximately 12 pounds per square inch; m) said coefficient of static friction is in the range of 0.2 to 1.0; and n) said density is in the range of approximately 83 kilograms per cubic meter to approximately 103 kilograms per cubic meter.

A further feature or aspect of an embodiment is believed at the time of the filing of this patent application to possibly reside broadly in the patient support arrangement, wherein: said pad comprises a viscoelastic material; said patient support arrangement further comprises securing straps attached to said pad and at least one body strap; said securing straps are configured to secure said pad to a medical procedure table; said securing straps each comprise a central portion, attached to a lower side of said pad, and fastener ends, each comprising a hook and loop fastener; said at least one body strap is configured to hold a patient down on a medical procedure table while a patient is in a Trendelenburg position; and said patient support arrangement comprises a kit.

One feature or aspect of an embodiment is believed at the time of the filing of this patent application to possibly reside broadly in a patient support arrangement configured to support and assist in holding a patient in a Trendelenburg position on an inclined medical procedure table, in which the head of the patient is disposed below the body of the patient, or in which the head of the patient is disposed above the body of the patient, or in which the right side of the patient is disposed above the left side or vice versa, or a combination of any of these positions, said patient support arrangement comprising: a pad comprising a viscoelastic polyurethane foam; said pad comprising a thickness and softness sufficient to permit deformation of said pad to a depth sufficient to assist in holding a patient on a medical procedure table during a medical procedure performed while a patient is in a Trendelenburg position; and said pad being configured to recover from a deformation at a rate in a range sufficient to maintain support and minimize disruptive movement of at least a portion of a body of a patient on a medical procedure table during a medical procedure performed while a patient is in a Trendelenburg position.

Another feature or aspect of an embodiment is believed at the time of the filing of this patent application to possibly reside broadly in the patient support arrangement, wherein said rate of recovery of said pad is insufficient to assist in causing disruptive movement of at least a portion of a body of a patient.

One feature or aspect of an embodiment is believed at the time of the filing of this patent application to possibly reside broadly in a system configured to minimize injuries on portions of a body of a patient on a surgical procedure table, such as a surgical operating table or a patient examination table, said system comprising a pad; said pad comprising: a coefficient of static friction bring sufficient to assist in minimizing unwanted movement of said pad on a surgical procedure table upon a patient being on a surgical procedure table; sufficient thickness to sufficiently cushion a body of a patient to: minimize bottoming out or prevent bottoming out, on a surgical procedure table, of one or more of portions of a body of a patient during positioning of a patient and during a surgical procedure, and to minimize injuries from pressure on a patient during a surgical procedure; and sufficient compliance to conform to a substantial portion of a body of a patient; sufficient thinness to stabilize a patient on a surgical procedure table; and said sufficient thickness and said sufficient compliance being configured to permit formation of a cavity in said pad of a depth sufficient to: assist in holding a patient on a surgical procedure table, and assist in minimizing undesired movement of a body of a patient on a surgical procure table, during a surgical procedure performed while a patient is in a surgical position; wherein said pad is configured to generate a sufficient static frictional force on skin of a patient on a surgical procedure table pad to stabilize a patient on a surgical procedure table pad; said system further comprising: a cleaning device configured to clean a scope during a medical procedure; said device comprising further: a casing housing a sponge and a heating element; and an attachment element connected to an outer surface of said casing and configured to removably attach said casing to a surgical implement; said surgical implement being configured to be operatively attachable to a patient; wherein said casing is configured to allow a lens of said scope to access said sponge and said heating element; and said pad provides an air flow sufficient to provide substantial air flow at at least a portion of a contact area of a patient, and prevent or minimize pressure injury to a patient.

One feature or aspect of an embodiment is believed at the time of the filing of this patent application to possibly reside broadly in a cleaning device configured for cleaning a scope during use, said cleaning device comprising: a casing housing a sponge and a heating element; and an attachment element connected to an outer surface of said casing and configured to removably attach said casing to a structure, wherein said casing is configured to permit a lens of said scope to access said sponge and said heating element.

Another feature or aspect of an embodiment is believed at the time of the filing of this patent application to possibly reside broadly in the cleaning device, wherein: said casing comprises a first depression formed in an upper surface of said casing and configured to receive said sponge therein and a second depression formed in said upper surface of said casing and configured to receive said heating element therein.

Yet another feature or aspect of an embodiment is believed at the time of the filing of this patent application to possibly reside broadly in the cleaning device, further comprising: a cover and a wiping element, wherein said cover is disposed over said second depression, wherein said cover comprises a first hole; wherein said wiping element comprises a second hole; and wherein said wiping element is disposed over said cover so that said first hole and said second hole are oriented to allow said scope to access said heating element.

Still another feature or aspect of an embodiment is believed at the time of the filing of this patent application to possibly reside broadly in the cleaning device, wherein said wiping element comprises a microfiber material.

A further feature or aspect of an embodiment is believed at the time of the filing of this patent application to possibly reside broadly in the cleaning device, wherein said sponge is impregnated with a cleaning medium.

Another feature or aspect of an embodiment is believed at the time of the filing of this patent application to possibly reside broadly in the cleaning device, wherein: said heating element comprises a heating coil and at least one battery operatively connected to said heating coil; said heating coil being configured to warm said lens of said scope when said scope is positioned in proximity to said heating coil.

Yet another feature or aspect of an embodiment is believed at the time of the filing of this patent application to possibly reside broadly in the cleaning device, further comprising one or more lights operatively connected to at least one battery.

Still feature or aspect of an embodiment is believed at the time of the filing of this patent application to possibly reside broadly in the cleaning device, wherein said heating element comprises a chemical pack.

A further feature or aspect of an embodiment is believed at the time of the filing of this patent application to possibly reside broadly in the cleaning device, wherein said attachment element comprises a snap member, said snap member being configured to removably attach said casing to said structure.

One feature or aspect of an embodiment is believed at the time of the filing of this patent application to possibly reside broadly in a cleaning kit for use a procedure, said cleaning kit comprising: a container housing said sponge configured to removably attach to said structure; a cover comprising a scope access hole configured to permit said scope to access said sponge; said cover positioned on an upper surface of said container; and a cleaner comprising an elongated body comprising a first end comprising a cleaning tip disposed thereon and a second end; said cleaner configured to be removably supported by at least one of said containers and said cover when not in use; wherein said cleaner being configured for insertion in said structure; and said cleaning tip being configured to contact cannula said structure upon said cleaner being inserted into said structure.

Another feature or aspect of an embodiment is believed at the time of the filing of this patent application to possibly reside broadly in the cleaning kit, wherein said elongated body of said cleaner further comprises: a solution container configured to receive a cleaning medium; and a valve disposed on said second end of said elongated body, with said valve being configured to dispense said cleaning medium during use.

Yet another feature or aspect of an embodiment is believed at the time of the filing of this patent application to possibly reside broadly in the cleaning kit, further comprising: at least one wiping element disposed on said cover; with said at least one wiping element being configured to allow said scope to access said sponge through said scope access hole.

Still another feature or aspect of an embodiment is believed at the time of the filing of this patent application to possibly reside broadly in the cleaning kit, further comprising: a heater disposed in said container; with said heater being configured to warm said scope inserted through said scope access hole.

A further feature or aspect of an embodiment is believed at the time of the filing of this patent application to possibly reside broadly in the cleaning kit, with said heater being configured to border a substantial portion of a perimeter of said sponge.

Another feature or aspect of an embodiment is believed at the time of the filing of this patent application to possibly reside broadly in the cleaning kit, wherein: said heater comprises a heating coil; at least one battery operatively connected to said heating coil; and with said heating coil being configured to warm said lens of said scope during use.

Yet another feature or aspect of an embodiment is believed at the time of the filing of this patent application to possibly reside broadly in the cleaning kit, further comprising one or more lights operatively connected to said at least one battery.

Still another feature or aspect of an embodiment is believed at the time of the filing of this patent application to possibly reside broadly in the cleaning kit, wherein said heater comprises a chemical pack.

A further feature or aspect of an embodiment is believed at the time of the filing of this patent application to possibly reside broadly in the cleaning kit, further comprising a snap member configured to attach said container to said structure.

One feature or aspect of an embodiment is believed at the time of the filing of this patent application to possibly reside broadly in a method of cleaning a scope during use comprising: inserting said structure into a patient; attaching said cleaning device to said structure; heating said heating element of said cleaning device to a desired temperature; inserting said scope into said structure; removing said scope from said structure; wiping said lens of said scope on said wiping element; and heating said lens of said scope with said heating element.

Another feature or aspect of an embodiment is believed at the time of the filing of this patent application to possibly reside broadly in the method, further comprising wiping said lens on said sponge.

One feature or aspect of an embodiment is believed at the time of the filing of this patent application to possibly reside broadly in a method of minimizing injuries caused by pressure on portions of a body of a patient in a Trendelenburg position on a surgical operating table, and minimizing unwanted movement of a patient in the Trendelenburg position on a surgical operating table by securing a patient to the surgical operating table, and also minimizing contamination in an operating room, by using a single-use Trendelenburg patient support system comprising: a lift sheet configured to lift and position a patient on a surgical operating table, body straps configured to hold a patient down on a surgical operating table, a single-use, viscoelastic Trendelenburg pad comprising a rectangular shape and viscoelastic polyurethane, and securing straps welded to said single-use, viscoelastic Trendelenburg pad, which said securing straps are configured to secure said single-use, viscoelastic Trendelenburg pad to rails of a surgical operating table, said method comprising the steps of: A) placing a longitudinal edge of said single-use, viscoelastic Trendelenburg pad adjacent and in alignment with a longitudinal edge of a surgical operating table having surgical operating table rails; B) placing a latitudinal edge of said single-use, viscoelastic Trendelenburg pad adjacent and in alignment with a latitudinal edge of said surgical operating table, and thus positioning said single-use, viscoelastic Trendelenburg pad in a position on said surgical operating table where the body of a patient will be lying; C) positioning said securing straps, extending from longitudinal edges of said single-use, viscoelastic Trendelenburg pad, to extend down and away from said single-use, viscoelastic Trendelenburg pad; D) attaching said securing straps to said surgical operating table rails; E) laying said lift sheet over said single-use, viscoelastic Trendelenburg pad; F) laying a patient in a supine position on said lift sheet and said single-use, viscoelastic Trendelenburg pad by positioning said patient so that the shoulders of said patient do not extend past edges of said single-use, viscoelastic Trendelenburg pad, and thereby deforming said single-use, viscoelastic Trendelenburg pad, which said single-use, viscoelastic Trendelenburg pad comprises: sufficient thickness and viscosity to sufficiently cushion the body of said patient to at least one of: minimize bottoming out and prevent bottoming out, on said medical procedure table, of one or more of the portions of the body of said patient during positioning of said patient and during a surgical procedure, and to minimize injuries from pressure during a surgical procedure performed while a patient is in lithotomy and Trendelenburg positions, and sufficient compliance to conform to a substantial portion of the body of said patient; G) lifting said lift sheet and thereby lifting said patient up and off said single-use, viscoelastic Trendelenburg pad to reposition said patient as or if needed; H) positioning the arms of said patient as or if needed; I) attaching said body straps around said patient and said surgical operating table; J) positioning the legs of said patient in a lithotomy position; K) adjusting the angle of inclination of said surgical operating table to orient said patient at an angle in a Trendelenburg position in which the head of said patient is disposed below the body of said patient, or in which the head of said patient is disposed above the body of said patient, or in which the right side of a patient is disposed above the left side or vice versa, or a combination of any of these positions; and L) assisting in substantially holding the body of said patient on said surgical operating table in said lithotomy and Trendelenburg positions using said single-use, viscoelastic Trendelenburg pad, which said single-use, viscoelastic Trendelenburg pad comprises: sufficient thinness to stabilize said patient on said surgical operating table upon said patient being in said lithotomy and Trendelenburg positions, and sufficient thickness and sufficient compliance to permit formation of a cavity in said single-use, viscoelastic Trendelenburg pad of a depth sufficient to at least one of: assist in holding said patient on said surgical operating table, and assist in minimizing undesired movement of the body of said patient on said surgical operating table, during a surgical procedure performed while said patient is in said lithotomy and Trendelenburg positions.

One feature or aspect of an embodiment is believed at the time of the filing of this patent application to possibly reside broadly in a method of minimizing injuries caused by pressure on portions of a body of a patient and minimizing unwanted movement of a patient upon securing a patient to a medical procedure table, such as a surgical operating table or a patient examination table, and upon placing said medical procedure table in an inclined position, using a patient support system comprising a viscoelastic pad and securing straps, said method comprising the steps of: A) positioning said viscoelastic pad in a position on said medical procedure table where the body of a patient will be lying; B) attaching said securing straps, connected to said viscoelastic pad, to said medical procedure table; C) positioning a patient on said viscoelastic pad and thereby deforming said viscoelastic pad, which said viscoelastic pad comprises: sufficient thickness and viscosity to sufficiently cushion the body of said patient to at least one of: minimize bottoming out and prevent bottoming out, on said medical procedure table, of one or more of the portions of the body of said patient during positioning of said patient and during a medical procedure, and to minimize injuries from pressure during a medical procedure, and sufficient compliance to conform to a substantial portion of the body of said patient; D) adjusting the angle of inclination of said medical procedure table to orient said patient at an angle in an inclined position, in which the head of said patient is disposed below the body of said patient, or in which the head of said patient is disposed above the body of said patient, or in which the right side of a patient is disposed above the left side or vice versa, or a combination of any of these positions; and E) assisting in substantially holding the body of said patient on said medical procedure table using said viscoelastic pad, of which said viscoelastic pad comprises: sufficient thinness to stabilize said patient on said medical procedure table upon said patient being in said inclined position, and sufficient thickness and sufficient compliance to permit formation of a cavity in said viscoelastic pad of a depth sufficient to at least one of: assist in holding a patient on a medical procedure table, and assist in minimizing undesired movement of the body of a patient on a medical procedure table, during a medical procedure performed while a patient is in an inclined position.

One feature or aspect of an embodiment is believed at the time of the filing of this patent application to possibly reside broadly in a method of minimizing injuries caused by pressure on portions of a body of a patient and minimizing unwanted movement of said patient upon said patient being on a medical procedure table, such as a surgical operating table or a patient examination table, and upon placing said medical procedure table in an inclined, Trendelenburg position, using a patient support system comprising a viscoelastic pad, said method comprising the steps of: A) positioning said viscoelastic pad in a position on said medical procedure table where the body of said patient will be lying; B) positioning said patient on said viscoelastic pad and thereby deforming said viscoelastic pad, which said viscoelastic pad comprises: sufficient thickness and viscosity to sufficiently cushion the body of said patient to at least one of: minimize bottoming out and prevent bottoming out, on said medical procedure table, of one or more of the portions of the body of said patient during positioning of said patient and during a medical procedure, and to minimize injuries from pressure during a medical procedure; and sufficient compliance to conform to a substantial portion of the body of said patient; C) adjusting the angle of inclination of said medical procedure table to orient said patient at an angle in said inclined, Trendelenburg position, in which the head of said patient is disposed below the body of said patient, or in which the head of said patient is disposed above the body of said patient, or in which the right side of said patient is disposed above the left side or vice versa, or a combination of any of these positions; and D) assisting in substantially holding the body of said patient on said medical procedure table using said viscoelastic pad, of which said viscoelastic pad comprises: sufficient thinness to stabilize said patient on said medical procedure table upon said patient being in said inclined, Trendelenburg position; and sufficient thickness and sufficient compliance to permit formation of a cavity in said viscoelastic pad of a depth sufficient to at least one of: assist in holding said patient on said medical procedure table, and assist in minimizing undesired movement of the body of said patient on said medical procedure table, during a medical procedure performed while said patient is in said inclined, Trendelenburg position.

One feature or aspect of an embodiment is believed at the time of the filing of this patent application to possibly reside broadly in a patient support arrangement comprising: a pad configured to be placed on a tiltable medical procedure table; said pad having a length sufficient to extend from about at least the thighs of a patient to about at least the shoulders of a patient to support the torso of a patient placed on said pad; said pad comprising a deformable material; said deformable material being configured to be deformable by the torso of a patient to form a depression in said pad, which depression provides a substantial portion of the holding forces which hold a patient generally in a desired position on said pad upon the medical procedure table being tilted at an angle and thus the torso of a patient being tilted at an angle with respect to the horizontal, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of any of a), b), c), and d); said deformable material has a rate of recovery sufficiently slow to maintain a depression in said pad for a desired period of time upon a change in a depression-generating force on said pad; and said pad is configured to distribute pressure forces across a substantial portion of the torso of a patient in contact with said pad to minimize injuries generated by concentration of pressure forces on at least one portion of the torso of the patient in contact with said pad during a medical procedure.

One feature or aspect of an embodiment is believed at the time of the filing of this patent application to possibly reside broadly in a method of positioning a patient on a medical procedure table comprising: positioning a pad, having a length sufficient to extend from about at least the thighs of a patient to about at least the shoulders of said patient, on a tiltable medical procedure table; positioning at least the torso of said patient on said pad, which pad comprises a deformable material, and thereby forming a depression in said deformable material, which deformable material has a rate of recovery sufficiently slow to maintain the depression in said pad for a desired period of time upon a change in a depression-generating force on said pad; tilting said medical procedure table at an angle, and thus positioning the torso of said patient at an angle with respect to the horizontal, such that the body of said patient is disposed in one of the positions a), b), c), d), and e), where a), b), c), d), and e) are: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of any of a), b), c), and d); and holding a patient generally in a desired position on said pad on said tilted medical procedure table, using said depression in said pad to provide a substantial portion of the holding forces, while distributing pressure forces across a substantial portion of the torso of said patient in contact with said pad to minimize injuries generated by concentration of pressure forces on at least one portion of the torso of said patient in contact with said pad during a medical procedure.

One feature or aspect of an embodiment is believed at the time of the filing of this patent application to possibly reside broadly in a patient support arrangement configured to support a body of a patient comprising thighs, shoulders, a torso, and a head, which patient has a right side and a left side, said patient support arrangement comprising:a pad arrangement configured to be placed on a tiltable medical procedure table; said pad arrangement having a length sufficient to extend from about at least the thighs of the patient to about at least the shoulders of the patient to support the torso of the patient placed on said pad arrangement; said pad arrangement comprising a deformable material; said deformable material being configured to be deformable by the torso of a patient to form a depression in said pad arrangement, which depression provides a substantial portion of holding forces which hold a patient generally in a desired position on said pad arrangement upon the medical procedure table being tilted at an angle and thus the torso of the patient being tilted at an angle with respect to horizontal, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of any of a), b), c), and d); said deformable material has a rate of recovery sufficiently slow to maintain a depression in said pad arrangement for a desired period of time upon a change in a depression-generating force on said pad arrangement; and said pad arrangement is configured to distribute pressure forces across a portion of the torso of a patient in contact with said pad arrangement sufficient to minimize injuries generated by concentration of pressure forces on at least one portion of the torso of the patient in contact with said pad arrangement during a medical procedure.

One feature or aspect of an embodiment is believed at the time of the filing of this patent application to possibly reside broadly in a method of positioning a patient on a medical procedure table configured to support a body of a patient comprising thighs, shoulders, a torso, and a head, which patient has a right side and a left side, said method comprising: positioning a pad arrangement, having a length sufficient to extend from about at least the thighs of a patient to about at least the shoulders of said patient, on a tiltable medical procedure table; positioning at least the torso of said patient on said pad arrangement, which pad arrangement comprises a deformable material, and thereby forming a depression in said deformable material, which deformable material has a rate of recovery sufficiently slow to maintain the depression in said pad arrangement for a desired period of time upon a change in a depression-generating force on said pad arrangement; tilting said medical procedure table at an angle, and thus positioning the torso of said patient at an angle with respect to horizontal, such that the body of said patient is disposed in one of the positions a), b), c), d), and e), where a), b), c), d), and e) are: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of any of a), b), c), and d); and holding a patient generally in a desired position on said pad arrangement on said tilted medical procedure table, using said depression in said pad arrangement to provide a substantial portion of holding forces, while distributing pressure forces across a portion of the torso of said patient in contact with said pad arrangement sufficient to minimize injuries generated by concentration of pressure forces on at least one portion of the torso of said patient in contact with said pad arrangement during a medical procedure.

One feature or aspect of an embodiment is believed at the time of the filing of this patent application to possibly reside broadly in a single use memory pad patient support system configured to minimize injuries caused by pressure on portions of a body of a patient and minimize unwanted movement of a patient upon a patient being in a Trendelenburg inclined position on a medical procedure table, such as a surgical operating table or a patient examination table, said single use memory pad patient support system comprising a pad; said pad comprising: a length and a width; said length being greater than said width; a viscoelastic polyurethane foam comprising a coefficient of static friction of at least 0.2 and said coefficient of static friction bring sufficient to assist in minimizing movement of said viscoelastic pad on said medical procedure table upon said patient being on said medical procedure table; sufficient thickness and viscosity to sufficiently cushion a body of a patient to: minimize bottoming out or prevent bottoming out, on said medical procedure table, of one or more of the portions of a body of a patient during positioning of a patient and during a medical procedure, and to minimize injuries from pressure during a medical procedure; and sufficient compliance to conform to a substantial portion of the body of a patient; sufficient thinness to stabilize a patient on a medical procedure table upon a patient being in an inclined, Trendelenburg position; and said sufficient thickness and said sufficient compliance to permit formation of a cavity in said pad of a depth sufficient to at least one of: assist in holding a patient on a medical procedure table, and assist in minimizing undesired movement of the body of a patient on a medical procure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left said of the patient, or d) the left side of the patient is disposed higher than the right said of the patient, or e) a combination of a), b), c), and d); wherein said viscoelastic memory foam pad is configured to generate a sufficient static frictional force on the skin of a patient on said medical procedure table pad; said pad is configured to generate a friction or friction-like force due to the formation of said cavity in said viscoelastic foam pad, and which force generated by said coefficient of static friction both together being sufficient to hold the body of the patient substantially static on a medical procedure table in the inclined Trendelenburg position; wherein a holding quality or ability between said pad and a patient is a combination of the coefficient of friction between the patient and the viscoelastic foam and the holding ability of the impression made by the patient in the viscoelastic foam; said holding quality or ability comprising an equivalent total frictional characteristic including the friction due to the coefficient of friction and the friction or friction-like force due to the viscoelastic foam deformation; said Trendelenburg pad comprises a viscoelastic foam in which an impression is formed and held in the foam at least for a time after the patient is moved from one position to another or from an operating table; and said equivalent total frictional characteristic being sufficient to hold a patient on a medical procedure table at an inclination of about 15 degrees.

One feature or aspect of an embodiment is believed at the time of the filing of this patent application to possibly reside broadly in a single use memory pad patient support system configured to minimize injuries caused by pressure on portions of a body of a patient and minimize unwanted movement of a patient upon a patient being in a Trendelenburg inclined position on a medical procedure table, such as a surgical operating table or a patient examination table, said single use memory pad patient support system comprising a pad; said pad comprising: a length and a width; said length being greater than said width; a viscoelastic polyurethane foam comprising a coefficient of static friction of at least 0.2 and said coefficient of static friction bring sufficient to assist in minimizing movement of said viscoelastic pad on said medical procedure table upon said patient being on said medical procedure table; sufficient thickness and viscosity to sufficiently cushion a body of a patient to: minimize bottoming out or prevent bottoming out, on said medical procedure table, of one or more of the portions of a body of a patient during positioning of a patient and during a medical procedure, and to minimize injuries from pressure during a medical procedure; and sufficient compliance to conform to a substantial portion of the body of a patient; sufficient thinness to stabilize a patient on a medical procedure table upon a patient being in an inclined, Trendelenburg position; and said sufficient thickness and said sufficient compliance to permit formation of a cavity in said pad of a depth sufficient to at least one of: assist in holding a patient on a medical procedure table, and assist in minimizing undesired movement of the body of a patient on a medical procure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left said of the patient, or d) the left side of the patient is disposed higher than the right said of the patient, or e) a combination of a), b), c), and d); wherein said viscoelastic memory foam pad is configured to generate a sufficient static frictional force on the skin of a patient on said medical procedure table pad; said pad is configured to generate a friction or friction-like force due to the formation of said cavity in said viscoelastic foam pad, and which force generated by said coefficient of static friction both together being sufficient to hold the body of the patient substantially static on a medical procedure table in the inclined Trendelenburg position; wherein a holding quality or ability between said pad and a patient is a combination of the coefficient of friction between the patient and the viscoelastic foam and the holding ability of the impression made by the patient in the viscoelastic foam; said holding quality or ability comprising an equivalent total frictional characteristic including the friction due to the coefficient of friction and the friction or friction-like force due to the viscoelastic foam deformation; said Trendelenburg pad comprises a viscoelastic foam in which an impression is formed and held in the foam at least for a time after the patient is moved from one position to another or from an operating table; and said equivalent total frictional characteristic being sufficient to hold a patient on a medical procedure table at an inclination of about 20 degrees.

One feature or aspect of an embodiment is believed at the time of the filing of this patent application to possibly reside broadly in a single use memory pad patient support system configured to minimize injuries caused by pressure on portions of a body of a patient and minimize unwanted movement of a patient upon a patient being in a Trendelenburg inclined position on a medical procedure table, such as a surgical operating table or a patient examination table, said single use memory pad patient support system comprising a pad; said pad comprising: a length and a width; said length being greater than said width; a viscoelastic polyurethane foam comprising a coefficient of static friction of at least 0.2 and said coefficient of static friction bring sufficient to assist in minimizing movement of said viscoelastic pad on said medical procedure table upon said patient being on said medical procedure table; sufficient thickness and viscosity to sufficiently cushion a body of a patient to: minimize bottoming out or prevent bottoming out, on said medical procedure table, of one or more of the portions of a body of a patient during positioning of a patient and during a medical procedure, and to minimize injuries from pressure during a medical procedure; and sufficient compliance to conform to a substantial portion of the body of a patient; sufficient thinness to stabilize a patient on a medical procedure table upon a patient being in an inclined, Trendelenburg position; and said sufficient thickness and said sufficient compliance to permit formation of a cavity in said pad of a depth sufficient to at least one of: assist in holding a patient on a medical procedure table, and assist in minimizing undesired movement of the body of a patient on a medical procure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left said of the patient, or d) the left side of the patient is disposed higher than the right said of the patient, or e) a combination of a), b), c), and d); wherein said viscoelastic memory foam pad is configured to generate a sufficient static frictional force on the skin of a patient on said medical procedure table pad; said pad is configured to generate a friction or friction-like force due to the formation of said cavity in said viscoelastic foam pad, and which force generated by said coefficient of static friction both together being sufficient to hold the body of the patient substantially static on a medical procedure table in the inclined Trendelenburg position; wherein a holding quality or ability between said pad and a patient is a combination of the coefficient of friction between the patient and the viscoelastic foam and the holding ability of the impression made by the patient in the viscoelastic foam; said holding quality or ability comprising an equivalent total frictional characteristic including the friction due to the coefficient of friction and the friction or friction-like force due to the viscoelastic foam deformation; said Trendelenburg pad comprises a viscoelastic foam in which an impression is formed and held in the foam at least for a time after the patient is moved from one position to another or from an operating table; and said equivalent total frictional characteristic being sufficient to hold a patient on a medical procedure table at an inclination of about 25 degrees.

One feature or aspect of an embodiment is believed at the time of the filing of this patent application to possibly reside broadly in a single use memory pad patient support system configured to minimize injuries caused by pressure on portions of a body of a patient and minimize unwanted movement of a patient upon a patient being in a Trendelenburg inclined position on a medical procedure table, such as a surgical operating table or a patient examination table, said single use memory pad patient support system comprising a pad; said pad comprising: a length and a width; said length being greater than said width; a viscoelastic polyurethane foam comprising a coefficient of static friction of at least 0.2 and said coefficient of static friction bring sufficient to assist in minimizing movement of said viscoelastic pad on said medical procedure table upon said patient being on said medical procedure table; sufficient thickness and viscosity to sufficiently cushion a body of a patient to: minimize bottoming out or prevent bottoming out, on said medical procedure table, of one or more of the portions of a body of a patient during positioning of a patient and during a medical procedure, and to minimize injuries from pressure during a medical procedure; and sufficient compliance to conform to a substantial portion of the body of a patient; sufficient thinness to stabilize a patient on a medical procedure table upon a patient being in an inclined, Trendelenburg position; and said sufficient thickness and said sufficient compliance to permit formation of a cavity in said pad of a depth sufficient to at least one of: assist in holding a patient on a medical procedure table, and assist in minimizing undesired movement of the body of a patient on a medical procure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left said of the patient, or d) the left side of the patient is disposed higher than the right said of the patient, or e) a combination of a), b), c), and d); wherein said viscoelastic memory foam pad is configured to generate a sufficient static frictional force on the skin of a patient on said medical procedure table pad; said pad is configured to generate a friction or friction-like force due to the formation of said cavity in said viscoelastic foam pad, and which force generated by said coefficient of static friction both together being sufficient to hold the body of the patient substantially static on a medical procedure table in the inclined Trendelenburg position; wherein a holding quality or ability between said pad and a patient is a combination of the coefficient of friction between the patient and the viscoelastic foam and the holding ability of the impression made by the patient in the viscoelastic foam; said holding quality or ability comprising an equivalent total frictional characteristic including the friction due to the coefficient of friction and the friction or friction-like force due to the viscoelastic foam deformation; said Trendelenburg pad comprises a viscoelastic foam in which an impression is formed and held in the foam at least for a time after the patient is moved from one position to another or from an operating table; and said equivalent total frictional characteristic being sufficient to hold a patient on a medical procedure table at an inclination of about 30 degrees.

One feature or aspect of an embodiment is believed at the time of the filing of this patent application to possibly reside broadly in a single use memory pad patient support system configured to minimize injuries caused by pressure on portions of a body of a patient and minimize unwanted movement of a patient upon a patient being in a Trendelenburg inclined position on a medical procedure table, such as a surgical operating table or a patient examination table, said single use memory pad patient support system comprising a pad; said pad comprising: a length and a width; said length being greater than said width; a viscoelastic polyurethane foam comprising a coefficient of static friction of at least 0.2 and said coefficient of static friction bring sufficient to assist in minimizing movement of said viscoelastic pad on said medical procedure table upon said patient being on said medical procedure table; sufficient thickness and viscosity to sufficiently cushion a body of a patient to: minimize bottoming out or prevent bottoming out, on said medical procedure table, of one or more of the portions of a body of a patient during positioning of a patient and during a medical procedure, and to minimize injuries from pressure during a medical procedure; and sufficient compliance to conform to a substantial portion of the body of a patient; sufficient thinness to stabilize a patient on a medical procedure table upon a patient being in an inclined, Trendelenburg position; and said sufficient thickness and said sufficient compliance to permit formation of a cavity in said pad of a depth sufficient to at least one of: assist in holding a patient on a medical procedure table, and assist in minimizing undesired movement of the body of a patient on a medical procure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left said of the patient, or d) the left side of the patient is disposed higher than the right said of the patient, or e) a combination of a), b), c), and d); wherein said viscoelastic memory foam pad is configured to generate a sufficient static frictional force on the skin of a patient on said medical procedure table pad; said pad is configured to generate a friction or friction-like force due to the formation of said cavity in said viscoelastic foam pad, and which force generated by said coefficient of static friction both together being sufficient to hold the body of the patient substantially static on a medical procedure table in the inclined Trendelenburg position; wherein a holding quality or ability between said pad and a patient is a combination of the coefficient of friction between the patient and the viscoelastic foam and the holding ability of the impression made by the patient in the viscoelastic foam; said holding quality or ability comprising an equivalent total frictional characteristic including the friction due to the coefficient of friction and the friction or friction-like force due to the viscoelastic foam deformation; said Trendelenburg pad comprises a viscoelastic foam in which an impression is formed and held in the foam at least for a time after the patient is moved from one position to another or from an operating table; and said equivalent total frictional characteristic being sufficient to hold a patient on a medical procedure table at an inclination of about 40 degrees.

One feature or aspect of an embodiment is believed at the time of the filing of this patent application to possibly reside broadly in a single use memory pad patient support system configured to minimize injuries caused by pressure on portions of a body of a patient and minimize unwanted movement of a patient upon a patient being in a Trendelenburg position on a medical procedure table, such as a surgical operating table or a patient examination table, said single use memory pad patient support system comprising a pad; said pad comprising: a viscoelastic polyurethane foam comprising a coefficient of static friction of at least 0.2 and said coefficient of static friction being sufficient to assist in minimizing movement of said viscoelastic pad on said medical procedure table upon said patient being on said medical procedure table; sufficient thickness and viscosity to sufficiently cushion a body of a patient to: minimize bottoming out or prevent bottoming out, on said medical procedure table, of one or more of the portions of a body of a patient during positioning of a patient and during a medical procedure, and to minimize injuries from pressure during a medical procedure; and sufficient compliance to conform to a substantial portion of the body of a patient; sufficient thinness to stabilize a patient on a medical procedure table upon a patient being in an inclined, Trendelenburg position; and sufficient thickness and sufficient compliance to permit formation of a cavity in said pad of a depth sufficient to at least one of: assist in holding a patient on a medical procedure table, and assist in minimizing undesired movement of the body of a patient on a medical procedure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of a), b), c), and d); wherein said viscoelastic memory foam pad is configured to generate a static frictional force on the skin of a patient on said medical procedure table pad, and further said pad is configured to generate a friction or friction-like force due to the formation of said cavity in said viscoelastic foam pad, and which force generated by said coefficient of static friction both together being sufficient to hold the body of the patient in the inclined Trendelenburg position; wherein a holding quality or ability between the Trendelenburg Pad and a patient is a combination of the coefficient of friction between the patient and the viscoelastic foam and the holding ability of the impression made by the patient in the viscoelastic foam, since the Trendelenburg pad comprises a viscoelastic foam in which an impression is formed and held in the foam at least for a time after the patient is moved from one position to another or from the operating table; an equivalent total frictional characteristic includes the friction due to the coefficient of friction and the friction or friction-like force due to the viscoelastic foam deformation; the equivalent total frictional characteristic includes the friction due to perpendicular force onto the viscoelastic pad from the body of the patient and a component of the force of gravity on the patient parallel to the surface of the operating table.

One feature or aspect of an embodiment is believed at the time of the filing of this patent application to possibly reside broadly in a method of minimizing injuries caused by pressure on portions of a body of a patient and minimizing unwanted movement of said patient upon said patient being in an inclined, Trendelenburg position on a medical procedure table, such as a surgical operating table or a patient examination table, using a patient support system comprising a pad comprising a viscoelastic memory material, said method comprising the steps of: A) positioning said patient support system in a position on said medical procedure table where the body of said patient will be lying; B) positioning said patient on said memory pad and deforming said memory pad and forming a slow-recovery cavity, which said memory pad comprises: a coefficient of static friction of at least 0.2; sufficient thickness to sufficiently cushion the body of said patient to: minimize bottoming out or prevent bottoming out, on said medical procedure table, of one or more of the portions of the body of said patient during positioning of said patient and during a medical procedure, and to minimize injuries from pressure during a medical procedure; and sufficient compliance to conform to a substantial portion of the body of said patient; C) adjusting the angle of inclination of said medical procedure table to orient said patient at an angle in said inclined, Trendelenburg position, in which the head of said patient is disposed below the body of said patient, or in which the head of said patient is disposed above the body of said patient, or in which the right side of said patient is disposed above the left side or vice versa, or a combination of these positions; and D) assisting in holding the body of said patient on said medical procedure table using said memory pad, which said memory pad comprises: sufficient thinness to stabilize said patient on said medical procedure table upon said patient being in said inclined, Trendelenburg position; and sufficient thickness and sufficient compliance to permit formation of said slow-recovery cavity in said memory pad of a depth sufficient to at least one of: assist in holding said patient on said medical procedure table, and assist in minimizing undesired movement of the body of said patient on said medical procedure table, during a medical procedure performed upon a patient being in an inclined, Trendelenburg position; wherein said viscoelastic memory foam pad generates a static frictional force on the skin of a patient on said medical procedure table pad, and further said pad generates a friction or friction-like force due to the formation of said cavity in said viscoelastic foam pad, and which force generated by said coefficient of static friction both together being sufficient to hold the body of the patient in the inclined Trendelenburg position; wherein a holding quality or ability between the Trendelenburg Pad and a patient is a combination of the coefficient of friction between the patient and the viscoelastic foam and the holding ability of the impression made by the patient in the viscoelastic foam, since the Trendelenburg pad comprises a viscoelastic foam in which an impression is formed and held in the foam at least for a time after the patient is moved from one position to another or from the operating table; an equivalent total frictional characteristic includes the friction due to the coefficient of friction and the friction or friction-like force due to the viscoelastic foam deformation; the equivalent total frictional characteristic includes the friction due to perpendicular force onto the viscoelastic pad from the body of the patient and a component of the force of gravity on the patient parallel to the surface of the operating table.

One feature or aspect of an embodiment is believed at the time of the filing of this patent application to possibly reside broadly in a method of minimizing injuries caused by pressure on portions of a body of a patient and minimizing unwanted movement of said patient upon said patient being table in an inclined, Trendelenburg position on a medical procedure table, such as a surgical operating table or a patient examination table, using a patient support system comprising a pad which pad comprises a memory material, said method comprising the steps of: A) positioning said pad comprising said memory material in a position on said medical procedure table where the body of said patient will be lying; B) positioning said patient on said pad and deforming said pad, which said pad comprises: a sufficient thickness to sufficiently cushion the body of said patient to at least one of: minimize bottoming out and prevent bottoming out, on said medical procedure table, of one or more of the portions of the body of said patient during positioning of said patient and during a medical procedure, and minimizing injuries from pressure during a medical procedure; and sufficient compliance to conform to a substantial portion of the body of said patient; C) adjusting the angle of inclination of said medical procedure table to orient said patient at an angle in said inclined, Trendelenburg position, in which the head of said patient is disposed below the body of said patient, or in which the head of said patient is disposed above the body of said patient, or in which the right side of said patient is disposed above the left side or vice versa, or a combination of any of these positions; and D) assisting in holding the body of said patient on said medical procedure table using said pad, which said pad comprises: sufficient thinness to stabilize said patient on said medical procedure table upon said patient being in said inclined, Trendelenburg position; a coefficient of static friction sufficient to assist in minimizing movement of said pad on said medical procedure table when said patient is on said medical procedure table upon being in the Trendelenburg position; a ball rebound in the range of approximately 0.1 percent to approximately 5 percent; and sufficient thickness and sufficient compliance to permit formation of a slow-recovery cavity in said pad to at least one of: assist in holding said patient on said medical procedure table, and assist in minimizing undesired movement of the body of said patient on said medical procedure table, during a medical procedure performed while said patient is in said inclined, Trendelenburg position; wherein said viscoelastic memory foam pad generates a static frictional force on the skin of a patient on said medical procedure table pad, and further said pad generates a friction or friction-like force due to the formation of said cavity in said viscoelastic foam pad, and which force generated by said coefficient of static friction both together being sufficient to hold the body of the patient in the inclined Trendelenburg position; wherein a holding quality or ability between the Trendelenburg Pad and a patient is a combination of the coefficient of friction between the patient and the viscoelastic foam and the holding ability of the impression made by the patient in the viscoelastic foam, since the Trendelenburg pad comprises a viscoelastic foam in which an impression is formed and held in the foam at least for a time after the patient is moved from one position to another or from the operating table; an equivalent total frictional characteristic includes the friction due to the coefficient of friction and the friction or friction-like force due to the viscoelastic foam deformation; the equivalent total frictional characteristic includes the friction due to perpendicular force onto the viscoelastic pad from the body of the patient and a component of the force of gravity on the patient parallel to the surface of the operating table.

One feature or aspect of an embodiment is believed at the time of the filing of this patent application to possibly reside broadly in a memory pad patient support system configured to minimize injuries caused by pressure on portions of a body of a patient and minimize unwanted movement of a patient upon a patient being in a Trendelenburg position on a medical procedure table, such as a surgical operating table or a patient examination table, said memory pad patient support system comprising a pad; said pad comprising: a retaining arrangement configured to at least assist in the retention of said pad arrangement on said medical table; a memory foam comprising a coefficient of static friction being sufficient to assist in minimizing movement of said memory pad on said medical procedure table upon said patient being on said medical procedure table; a sufficient thickness and a sufficient viscosity to sufficiently cushion a body of a patient to: minimize bottoming out or prevent bottoming out, on said medical procedure table, of one or more of the portions of a body of a patient during positioning of a patient on said medical procedure table and during a medical procedure, and to minimize injuries from pressure on body of a patient during a medical procedure; and sufficient compliance to conform to a substantial portion of the body of a patient; sufficient thinness to stabilize a patient on a medical procedure table upon a patient being in an inclined, Trendelenburg position; and sufficient thickness and sufficient compliance to permit formation of a cavity in said pad of a depth sufficient to at least one of: assist in holding a patient on a medical procedure table, and assist in minimizing undesired movement of the body of a patient on a medical procedure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of a), b), c), or d); said memory foam comprising a substantially viscoelastic polyurethane foam; and said pad comprising a ball rebound sufficiently low to minimize unwanted movement of the body of a patient on a medical procedure table during a medical procedure.

One feature or aspect of an embodiment is believed at the time of the filing of this patent application to possibly reside broadly in a method of minimizing injuries caused by pressure on portions of a body of a patient and minimizing unwanted movement of said patient upon said patient being table in an inclined, Trendelenburg position on a medical procedure table, such as a surgical operating table or a patient examination table, using a patient support system comprising a pad which pad comprises a memory material, which memory material comprises a substantially viscoelastic polyurethane foam comprising a ball rebound sufficiently low to minimize unwanted movement of the body of said patient on said medical procedure table during a medical procedure, said method comprising the steps of: A) positioning said pad comprising said memory material in a position on said medical procedure table where the body of said patient will be lying; B) positioning said patient on said pad and deforming said pad, which said pad comprises: a retaining arrangement configured to at least assist in the retention of said pad arrangement on said medical table; a sufficient thickness to sufficiently cushion the body of said patient to at least one of: minimize bottoming out and prevent bottoming out, on said medical procedure table, of one or more of the portions of the body of said patient during positioning of said patient and during a medical procedure, and minimizing injuries from pressure during a medical procedure; and sufficient compliance to conform to a substantial portion of the body of said patient; C) adjusting the angle of inclination of said medical procedure table to orient said patient at an angle in said inclined, Trendelenburg position, in which the head of said patient is disposed below the body of said patient, or in which the head of said patient is disposed above the body of said patient, or in which the right side of said patient is disposed above the left side or vice versa, or a combination of any of these positions; and D) assisting in holding the body of said patient on said medical procedure table using said pad, wherein said pad comprises: sufficient thinness to stabilize said patient on said medical procedure table upon said patient being in said inclined, Trendelenburg position; sufficient thickness, sufficient compliance, and sufficient viscosity to permit formation of a slow-recovery cavity in said pad to at least one of: assist in holding said patient on said medical procedure table, and assist in minimizing undesired movement of the body of said patient on said medical procedure table, during a medical procedure performed while said patient is in said inclined, Trendelenburg position; and forming a cavity in said pad of a depth sufficient to at least one of: assist in holding a patient on a medical procedure table, and assist in minimizing undesired movement of the body of a patient on a medical procedure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of a), b), c), or d).

One feature or aspect of an embodiment is believed at the time of the filing of this patent application to possibly reside broadly in a memory pad patient support system configured to minimize injuries caused by pressure on portions of a body of a patient and minimize unwanted movement of a patient upon a patient being in a Trendelenburg position on a medical procedure table, such as a surgical operating table or a patient examination table, said memory pad patient support system comprising a pad; said pad comprising: a retaining arrangement configured to at least assist in the retention of said pad arrangement on said medical table; a memory material; a sufficient thickness and a sufficient viscosity to sufficiently cushion a body of a patient to: minimize bottoming out or prevent bottoming out, on said medical procedure table, of one or more of the portions of a body of a patient during positioning of a patient on said medical procedure table and during a medical procedure, and to minimize injuries from pressure on body of a patient during a medical procedure; and sufficient compliance to conform to a portion of the body of a patient which deforms said pad; sufficient thinness to stabilize a patient on a medical procedure table upon a patient being in an inclined, Trendelenburg position; and sufficient thickness and sufficient compliance to permit formation of a cavity in said pad of a depth sufficient to at least one of: assist in holding a patient on a medical procedure table, and assist in minimizing undesired movement of the body of a patient on a medical procedure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of a), b), c), or d); said memory foam comprising a substantially viscoelastic polyurethane foam; and said pad comprising a ball rebound sufficiently low to minimize unwanted movement of the body of a patient on a medical procedure table during a medical procedure.

One feature or aspect of an embodiment is believed at the time of the filing of this patent application to possibly reside broadly in a memory pad patient support system configured to minimize injuries caused by pressure on portions of a body of a patient and minimize unwanted movement of a patient upon a patient being in a Trendelenburg position on a medical procedure table, such as a surgical operating table or a patient examination table, said memory pad patient support system comprising a pad; said pad comprising: a retaining arrangement configured to at least assist in the retention of said pad arrangement on said medical table; a sufficient thickness and a sufficient viscosity to form a slow-recovering cavity to sufficiently cushion a body of a patient to: minimize bottoming out or prevent bottoming out, on said medical procedure table, of one or more of the portions of a body of a patient during positioning of a patient on said medical procedure table during a medical procedure, and to minimize injuries from pressure on body of a patient during a medical procedure; and sufficient compliance to conform to a portion of the body of a patient which deforms said pad; sufficient thinness to stabilize a patient on a medical procedure table upon a patient being in an inclined, Trendelenburg position; and sufficient thickness and sufficient compliance to permit formation of a cavity in said pad of a depth sufficient to at least one of: assist in holding a patient on a medical procedure table, and assist in minimizing undesired movement of the body of a patient on a medical procedure table, during a medical procedure performed while said patient is in an inclined Trendelenburg position, such that: a) the head of the patient is disposed lower than the torso of the patient, or b) the head of the patient is disposed higher than the torso of the patient, or c) the right side of the patient is disposed higher than the left side of the patient, or d) the left side of the patient is disposed higher than the right side of the patient, or e) a combination of a), b), c), or d); said memory foam comprising a substantially viscoelastic polyurethane foam; and said pad comprising a ball rebound sufficient to minimize unwanted movement of the body of a patient on a medical procedure table during a medical procedure.

One feature or aspect of an embodiment is believed at the time of the filing of this patent application to possibly reside broadly in a surgical patient single use, open cell foam support pad comprising a viscoelastic memory foam which has a coefficient of friction between said viscoelastic memory foam and a surface of a surgical procedure table configured to hold said pad on a surgical procedure table, which surgical patient single use, open cell foam support pad comprises viscoelastic memory foam which is deformable so as to conform to a body of a patient, and which thereby assists in holding a patient in an inclined, Trendelenburg, position on an inclined surgical procedure table, and prevents or minimizes bottoming out on a surgical procedure table of a portion of a body of a patient to reduce injury caused by pressure to a patient during a surgical procedure, where an inclined, Trendelenburg, position includes an orientation in which a head of a patient is disposed either a) below or b) above a body of a patient or c) a right side of a patient is disposed higher than a left side of a patient, or d) a left side of a patient is disposed higher than a right side of a patient, or e) a combination of a), b), c), and d); said surgical patient single use, open cell foam support pad further comprising: a flat top surface and a flat bottom surface; a single layer of viscoelastic memory foam; said viscoelastic memory foam is uncovered on its top and bottom; said single layer of said viscoelastic memory foam extends all the way through said surgical patient single use, open cell foam support pad from a patient's skin to a surface of a surgical procedure table between the flat top surface and the flat bottom surface of said surgical patient single use, open cell foam support pad; said viscoelastic memory foam comprising a coefficient of static friction in the range of 0.2 to 1.0 and said coefficient of static friction being at a value in said range sufficient to assist in minimizing movement of said surgical patient single use, open cell foam support pad on a surgical procedure table upon a patient being on a surgical procedure table; sufficient thickness and viscosity to sufficiently cushion a body of a patient to: minimize bottoming out or prevent bottoming out, on a surgical procedure table, of one or more of portions of a body of a patient during positioning of a patient and during a surgical procedure, and to minimize injuries from pressure on a patient during a surgical procedure; sufficient compliance to conform to a substantial portion of a body of a patient; sufficient thinness to stabilize a patient on a surgical procedure table; and said sufficient thickness and said sufficient compliance being configured to permit formation of a cavity in said surgical patient single use, open cell foam support pad of a depth sufficient to at least one of: assist in holding a patient on a surgical procedure table, and assist in minimizing undesired movement of a body of a patient on a surgical procedure table, during a surgical procedure performed while a patient is in an surgical position; said surgical patient single use, open cell foam support pad being configured to generate a sufficient static frictional force on skin of a patient on said surgical patient single use, open cell foam support pad to stabilize a patient on said surgical patient single use, open cell foam support pad; said surgical patient single use, open cell foam support pad is configured to generate a friction or friction-like force due to formation of a cavity in said surgical patient single use, open cell foam support pad, and which force generated by said coefficient of static friction both together being sufficient to hold a body of a patient substantially static on a surgical procedure table; wherein a holding quality or ability between said surgical patient single use, open cell foam support pad and a patient is a combination of the coefficient of friction between a patient and said viscoelastic memory foam and the holding ability of the impression made by a patient in said viscoelastic memory foam; said holding quality or ability comprising an equivalent total frictional characteristic including the friction due to the coefficient of friction and the friction or friction-like force due to the viscoelastic memory foam deformation; said viscoelastic memory foam pad comprises a viscoelastic foam in which an impression is formed and held in the foam at least for a time after a patient is moved from one position to another or from a surgical procedure table; said open cell foam also provides an air flow sufficient to provide substantial air flow about a patient; and wherein said air flow of said open cell foam is in the range of one of i) and ii), wherein i) and ii) are: i) 0.1 to 3.0 cubic foot per minute; and ii) 0.3 to 1.0 cubic foot per minute.

One feature or aspect of an embodiment is believed at the time of the filing of this patent application to possibly reside broadly in a single use pad patient support system configured to minimize injuries caused by pressure on portions of a body of a patient and minimize unwanted movement of a patient upon a patient being positioned on a surgical procedure table, such as a surgical operating table or a patient examination table, said single use memory pad patient support system comprising a pad; said pad comprising: a viscoelastic, polyurethane open cell foam comprising a coefficient of static friction in the range of 0.2 to 1.0 and said coefficient of static friction being at a value in said range sufficient to assist in minimizing movement of said pad on a surgical procedure table upon a patient being on a surgical procedure table; sufficient thickness and viscosity to sufficiently cushion a body of a patient to: minimize bottoming out or prevent bottoming out, on a surgical procedure table, of one or more of portions of a body of a patient during positioning of a patient and during a surgical procedure, and to minimize injuries from pressure on a patient during a surgical procedure; and sufficient compliance to conform to a substantial portion of a body of a patient; sufficient thinness to stabilize a patient on a surgical procedure table; and said sufficient thickness and said sufficient compliance being configured to permit formation of a cavity in said pad of a depth sufficient to at least one of: assist in holding a patient on a surgical procedure table, and assist in minimizing undesired movement of a body of a patient on a surgical procedure table, during a surgical procedure performed while a patient is in a surgical position; said pad being configured to generate a sufficient static frictional force on skin of a patient on said pad to stabilize a patient on said pad; said pad being configured to generate a friction or friction-like force due to formation of a cavity in said pad, and which force generated by said coefficient of static friction both together being sufficient to hold a body of a patient substantially static on a surgical procedure table; wherein a holding quality or ability between said pad and a patient is a combination of the coefficient of friction between a patient and said pad and the holding ability of the impression made by a patient in said pad; said holding quality or ability comprising an equivalent total frictional characteristic including the friction due to the coefficient of friction and the friction or friction-like force due to the pad deformation; said pad further comprising a viscoelastic foam in which an impression is formed and held in the foam at least for a time after a patient is moved from one position to another or from an operating table; and said viscoelastic foam also provides an air flow sufficient to provide substantial air flow at the skin of a patient in contact with said viscoelastic foam; and wherein said air flow of said open cell foam is in the range of one of i) and ii), wherein i) and ii) are: i) 0.1 to 3.0 cubic foot per minute; and ii) 0.3 to 1.0 cubic foot per minute.

One feature or aspect of an embodiment is believed at the time of the filing of this patent application to possibly reside broadly in a patient support system configured to minimize injuries caused by pressure on portions of a body of a patient and minimize unwanted movement of a patient upon a patient being in surgical position on a surgical procedure table, such as a surgical operating table or a patient examination table, said patient support system comprising a pad; said pad comprising: a viscoelastic, polyurethane open cell foam comprising a coefficient of static friction in the range of 0.2 to 1.0 and said coefficient of static friction bring sufficient to assist in minimizing movement of said pad on a surgical procedure table upon a patient being on a surgical procedure table; sufficient thickness to sufficiently cushion a body of a patient to: minimize bottoming out or prevent bottoming out, on a surgical procedure table, of one or more of portions of a body of a patient during positioning of a patient and during a surgical procedure, and to minimize injuries from pressure on a patient during a surgical procedure; and sufficient compliance to conform to a substantial portion of a body of a patient; sufficient thinness to stabilize a patient on a surgical procedure table; and said sufficient thickness and said sufficient compliance being configured to permit formation of a cavity in said pad of a depth sufficient to at least one of: assist in holding a patient on a surgical procedure table, and assist in minimizing undesired movement of a body of a patient on a surgical procure table, during a surgical procedure performed while a patient is in a surgical position; wherein said pad is configured to generate a sufficient static frictional force on skin of a patient on a surgical procedure table pad to stabilize a patient on a surgical procedure table pad; said pad is configured to generate a friction or friction-like force due to the formation of a cavity in said viscoelastic foam pad, and which force generated by said coefficient of static friction both together being sufficient to hold a body of a patient substantially static on a surgical procedure table; wherein a holding quality or ability between said pad and a patient is a combination of the coefficient of friction between a patient and said pad and the holding ability of the impression made by a patient in said pad; said holding quality or ability comprising an equivalent total frictional characteristic including the friction due to the coefficient of friction and the friction or friction-like force due to the pad deformation; and said viscoelastic foam pad comprises a viscoelastic foam in which an impression is formed and held in the foam at least for a time after a patient is moved from one position to another or from a surgical table; in which said viscoelastic, polyurethane open cell foam also provides an air flow sufficient to provide substantial air flow at at least a portion of a contact area of a patient; and wherein said air flow of said viscoelastic foam is in the range of one of i) and ii), wherein i) and ii) are: i) 0.1 to 3.0 cubic foot per minute; and ii) 0.3 to 1.0 cubic foot per minute.

One feature or aspect of an embodiment is believed at the time of the filing of this patent application to possibly reside broadly in a cleaning device configured for cleaning a surgical scope during minimally invasive surgery comprising: a casing defining an interior, the casing comprising an upper surface comprising at least one opening for accessing the interior, a bottom surface, and a sidewall extending therebetween; a sponge and a heating element disposed in the interior of the casing; and an attachment element comprising a snap member integrally molded with and extending from the casing, the snap member comprising a first arm and a second arm extending from a sidewall of the casing defining a cavity having a partially circular cross section configured to receive and engage an outer surface of a tubular portion of a trocar to removably attach the casing to the trocar in a position in which a central longitudinal axis of the trocar is spaced apart from the interior of the casing, wherein the attachment element supports the casing, so that a line normal to the bottom surface and passing through the opening of the casing is parallel to and a fixed distance from the central longitudinal axis of the trocar, and wherein the casing is configured to allow a lens of the surgical scope to access the sponge and the heating element.

Another feature or aspect of an embodiment is believed at the time of the filing of this patent application to possibly reside broadly in the cleaning device, wherein: the interior of the casing comprises a first chamber, the sponge being disposed therein, and a second chamber separate from the first chamber, wherein the heating element is disposed in the second chamber.

Yet another feature or aspect of an embodiment is believed at the time of the filing of this patent application to possibly reside broadly in the cleaning device, wherein the upper surface of the casing further comprises at least one recess, the device further comprising a wiping element disposed in the at least one recess.

Still another feature or aspect of an embodiment is believed at the time of the filing of this patent application to possibly reside broadly in the cleaning device, wherein the wiping element is comprised of a microfiber material.

A further feature or aspect of an embodiment is believed at the time of the filing of this patent application to possibly reside broadly in the cleaning device, wherein the sponge is impregnated with a cleaning medium.

Another feature or aspect of an embodiment is believed at the time of the filing of this patent application to possibly reside broadly in the cleaning device, wherein: the heating element is comprised of a heating coil and at least one battery operatively connected to the heating coil, wherein the heating coil is configured to warm the lens of the surgical scope when the surgical scope is positioned in proximity to the heating coil.

Yet another feature or aspect of an embodiment is believed at the time of the filing of this patent application to possibly reside broadly in the cleaning device, further comprising one or more lights operatively connected to the at least one battery.

Still another feature or aspect of an embodiment is believed at the time of the filing of this patent application to possibly reside broadly in the cleaning device, wherein the heating element is comprised of a chemical pack.

One feature or aspect of an embodiment is believed at the time of the filing of this patent application to possibly reside broadly in a cleaning kit for use during minimally invasive surgery comprising: a container housing a sponge configured to removably attach to a trocar; a cover comprising a scope access hole configured to permit a surgical scope to access the sponge, the cover positioned on an upper surface of the container; and a cannula cleaner comprising an elongated body having a first end having a cleaning tip disposed thereon and a second end, the cannula cleaner configured to be removably supported by at least one of the container and the cover when not in use, wherein the cannula cleaner is configured for insertion in a cannula of the trocar and the cleaning tip is configured to contact the cannula when the cannula cleaner is inserted into the cannula.

Another feature or aspect of an embodiment is believed at the time of the filing of this patent application to possibly reside broadly in the cleaning kit, wherein the elongated body of the cannula cleaner further comprises: a solution container configured to receive a cleaning medium; and a valve disposed on the second end of the elongated body, wherein the valve is configured to dispense the cleaning medium during surgery.

Yet another feature or aspect of an embodiment is believed at the time of the filing of this patent application to possibly reside broadly in the cleaning kit, further comprising: at least one wiping element disposed on the cover, wherein the at least one wiping element is configured to allow the surgical scope to access the sponge through the scope access hole.

Still another feature or aspect of an embodiment is believed at the time of the filing of this patent application to possibly reside broadly in the cleaning kit, further comprising: a heater disposed in the container, wherein the heater is configured to warm the surgical scope inserted through the scope access hole.

A further feature or aspect of an embodiment is believed at the time of the filing of this patent application to possibly reside broadly in the cleaning kit, wherein the heater is configured to border a substantial portion of a perimeter of the sponge.

Another feature or aspect of an embodiment is believed at the time of the filing of this patent application to possibly reside broadly in the cleaning kit, wherein the heater comprises a heating coil and at least one battery operatively connected to the heating coil, and wherein the heating coil is configured to warm a lens of the surgical scope during surgery.

Yet another feature or aspect of an embodiment is believed at the time of the filing of this patent application to possibly reside broadly in the cleaning kit, further comprising one or more lights operatively connected to the at least one battery.

Still another feature or aspect of an embodiment is believed at the time of the filing of this patent application to possibly reside broadly in the cleaning kit, wherein the heater comprises a chemical pack.

A further feature or aspect of an embodiment is believed at the time of the filing of this patent application to possibly reside broadly in the cleaning kit, further comprising a snap member configured to attach the container to the trocar.

One feature or aspect of an embodiment is believed at the time of the filing of this patent application to possibly reside broadly in a trocar assembly for introducing a surgical scope to a body during a minimally invasive surgery, the assembly comprising: a trocar comprising a tubular portion comprising an outer surface; and a cleaning device mounted to the trocar configured for cleaning the surgical scope, the cleaning device comprising: a casing defining an interior, the casing comprising an upper surface comprising at least one opening for accessing the interior, a bottom surface, and a sidewall extending therebetween; a sponge and a heating element disposed in the interior of the casing; and an attachment element comprising a snap member integrally molded with and extending from the casing, the snap member comprising a first arm and a second arm extending from a sidewall of the casing defining a cavity having a partially circular cross section configured to receive and engage the outer surface of the tubular portion of the trocar to removably attach the casing to the trocar in a position in which a central longitudinal axis of the trocar is spaced apart from the interior of the casing, wherein the attachment element supports the casing, so that a line normal to the bottom surface and passing through the opening of the casing is parallel to and a fixed distance from the central longitudinal axis of the trocar, and wherein the casing is configured to allow a lens of the surgical scope to access the sponge and the heating element.

Another feature or aspect of an embodiment is believed at the time of the filing of this patent application to possibly reside broadly in the trocar assembly, wherein the interior of the casing comprises a first chamber, the sponge being disposed therein, and a second chamber separate from the first chamber, wherein the heating element is disposed in the second chamber.

Yet another feature or aspect of an embodiment is believed at the time of the filing of this patent application to possibly reside broadly in the system, wherein the heating element comprises a heating coil and at least one battery operatively connected to the heating coil, and wherein the heating coil is configured to warm the lens of the surgical scope when the surgical scope is positioned in proximity to the heating coil.

Still another feature or aspect of an embodiment is believed at the time of the filing of this patent application to possibly reside broadly in the cleaning device, wherein each arm of the snap member comprises an inwardly directed protrusion extending into the cavity for engaging the trocar to maintain the trocar within the cavity.

A further feature or aspect of an embodiment is believed at the time of the filing of this patent application to possibly reside broadly in the cleaning device, wherein the attachment element maintains the casing in an upright orientation relative to the trocar, such that a longitudinal axis of the casing is parallel to the central longitudinal axis of at least a portion of the trocar engaged to the attachment extension.

One feature or aspect of an embodiment is believed at the time of the filing of this patent application to possibly reside broadly in a cleaning device configured for cleaning a surgical scope during minimally invasive surgery comprising: a casing housing a sponge and a heating element; and an attachment element connected to an outer surface of the casing and configured to removably attach the casing to a trocar, wherein the casing is configured to allow a lens of the surgical scope to access the sponge and the heating element.

Another feature or aspect of an embodiment is believed at the time of the filing of this patent application to possibly reside broadly in the cleaning device, wherein: the casing comprises a first depression formed in an upper surface of the casing and configured to receive the sponge therein and a second depression formed in an upper surface of the casing and configured to receive the heating element therein.

Yet another feature or aspect of an embodiment is believed at the time of the filing of this patent application to possibly reside broadly in the cleaning device, further comprising: a cover and a wiping element, wherein the cover is disposed over the second depression, wherein the cover comprises a first hole; wherein the wiping element comprises a second hole; and wherein the wiping element is disposed over the cover so that the first hole and the second hole are oriented to allow the surgical scope to access the heating element.

Still another feature or aspect of an embodiment is believed at the time of the filing of this patent application to possibly reside broadly in the cleaning device, wherein the wiping element is comprised of a microfiber material.

A further feature or aspect of an embodiment is believed at the time of the filing of this patent application to possibly reside broadly in the cleaning device, wherein the sponge is impregnated with a cleaning medium.

Another feature or aspect of an embodiment is believed at the time of the filing of this patent application to possibly reside broadly in the cleaning device, wherein: the heating element is comprised of a heating coil and at least one battery operatively connected to the heating coil, wherein the heating coil is configured to warm the lens of the surgical scope when the surgical scope is positioned in proximity to the heating coil.

Yet another feature or aspect of an embodiment is believed at the time of the filing of this patent application to possibly reside broadly in the cleaning device, further comprising one or more lights operatively connected to the at least one battery.

Still another feature or aspect of an embodiment is believed at the time of the filing of this patent application to possibly reside broadly in the cleaning device, wherein the heating element is comprised of a chemical pack.

A further feature or aspect of an embodiment is believed at the time of the filing of this patent application to possibly reside broadly in the cleaning device, wherein the attachment element is a snap member, the snap member being configured to removably attach the casing to the trocar.

One feature or aspect of an embodiment is believed at the time of the filing of this patent application to possibly reside broadly in a cleaning kit for use during minimally invasive surgery comprising: a container housing a sponge configured to removably attach to a trocar; a cover comprising a scope access hole configured to permit a surgical scope to access the sponge, the cover positioned on an upper surface of the container; and a cannula cleaner comprising an elongated body having a first end having a cleaning tip disposed thereon and a second end, the cannula cleaner configured to be removably supported by at least one of the container and the cover when not in use, wherein the cannula cleaner is configured for insertion in a cannula of the trocar and the cleaning tip is configured to contact the cannula when the cannula cleaner is inserted into the cannula.

Another feature or aspect of an embodiment is believed at the time of the filing of this patent application to possibly reside broadly in the cleaning kit, wherein the elongated body of the cannula cleaner further comprises: a solution container configured to receive a cleaning medium; and a valve disposed on the second end of the elongated body, wherein the valve is configured to dispense the cleaning medium during surgery.

Yet another feature or aspect of an embodiment is believed at the time of the filing of this patent application to possibly reside broadly in the cleaning kit, further comprising: at least one wiping element disposed on the cover, wherein the at least one wiping element is configured to allow the surgical scope to access the sponge through the scope access hole.

Still another feature or aspect of an embodiment is believed at the time of the filing of this patent application to possibly reside broadly in the cleaning kit, further comprising: a heater disposed in the container; wherein the heater is configured to warm the surgical scope inserted through the scope access hole.

A further feature or aspect of an embodiment is believed at the time of the filing of this patent application to possibly reside broadly in the cleaning kit, wherein the heater is configured to border a substantial portion of a perimeter of the sponge.

Another feature or aspect of an embodiment is believed at the time of the filing of this patent application to possibly reside broadly in the cleaning kit, wherein the heater comprises a heating coil and at least one battery operatively connected to the heating coil, and wherein the heating coil is configured to warm a lens of the surgical scope during surgery.

Yet another feature or aspect of an embodiment is believed at the time of the filing of this patent application to possibly reside broadly in the cleaning kit, further comprising one or more lights operatively connected to the at least one battery.

Still another feature or aspect of an embodiment is believed at the time of the filing of this patent application to possibly reside broadly in the cleaning kit, wherein the heater comprises a chemical pack.

A further feature or aspect of an embodiment is believed at the time of the filing of this patent application to possibly reside broadly in the cleaning kit, further comprising a snap member configured to attach the container to the trocar.

One feature or aspect of an embodiment is believed at the time of the filing of this patent application to possibly reside broadly in a method of cleaning a surgical scope during minimally invasive surgery comprising: inserting a trocar into a patient; attaching a cleaning device according to the present application to the trocar; heating a heating element of the cleaning device to a desired temperature; inserting the surgical scope from the trocar; wiping a lens of the surgical scope on the wiping element; and heating the lens of the surgical scope with the heating element.

Another feature or aspect of an embodiment is believed at the time of the filing of this patent application to possibly reside broadly in the method, further comprising wiping the lens on the sponge.

The appended drawings in their entirety, including all dimensions, proportions and/or shapes in at least one embodiment of the invention, are accurate and are hereby included by reference into this specification.

The embodiments of the invention described herein above in the context of the preferred embodiments are not to be taken as limiting the embodiments of the invention to all of the provided details thereof, since modifications and variations thereof may be made without departing from the spirit and scope of the embodiments of the invention.

What is claimed is:

1. A system configured to minimize injuries on portions of a body of a patient on a surgical procedure table, such as a surgical operating table or a patient examination table, said system comprising a single use pad patient support system configured to minimize injuries caused by pressure on portions of a body of a patient and minimize unwanted movement of a patient upon a patient being positioned on a surgical procedure table, such as a surgical operating table or a patient examination table,
said single use memory pad patient support system comprising a pad;
said pad comprising:
a viscoelastic, polyurethane open cell foam comprising a coefficient of static friction in the range of 0.2 to 1.0 and said coefficient of static friction being at a value in said range sufficient to assist in minimizing movement of said pad on a surgical procedure table upon a patient being on a surgical procedure table;
sufficient thickness and viscosity to sufficiently cushion a body of a patient to: minimize bottoming out or prevent bottoming out, on a surgical procedure table, of one or more of portions of a body of a patient during positioning of a patient and during a surgical procedure, and to minimize injuries from pressure on a patient during a surgical procedure; and
sufficient compliance to conform to a substantial portion of a body of a patient;
sufficient thinness to stabilize a patient on a surgical procedure table; and
said sufficient thickness and said sufficient compliance being configured to permit formation of a cavity in said pad of a depth sufficient to at least one of: assist in holding a patient on a surgical procedure table, and assist in minimizing undesired movement of a body of a patient on a surgical procedure table, during a surgical procedure performed while a patient is in a surgical position;
said pad being configured to generate a sufficient static frictional force on skin of a patient on said pad to stabilize a patient on said pad;
said pad being configured to generate a friction or friction-like force due to formation of a cavity in said pad, and which force generated by said coefficient of static friction both together being sufficient to hold a body of a patient substantially static on a surgical procedure table;
said system further comprising:
a cleaning device configured to clean an instrument during a medical procedure;
said device comprising further:
a casing housing a cleaning arrangement and a heating element; and
an attachment element connected to an outer surface of said casing and configured to removably attach said casing to a surgical implement;
said surgical implement being configured to be operatively attachable to a patient;
wherein said casing is configured to allow said instrument to access said cleaning arrangement and said heating element;
and further wherein a holding quality or ability between said pad and a patient is a combination of the coefficient of friction between a patient and said pad and the holding ability of the impression made by a patient in said pad;
said holding quality or ability comprising an equivalent total frictional characteristic including the friction due to the coefficient of friction and the friction or friction-like force due to the pad deformation;
said pad further comprising a viscoelastic foam in which an impression is formed and held in the foam at least for a time after a patient is moved from one position to another or from an operating table;
and said viscoelastic foam also provides an air flow sufficient to provide substantial air flow at the skin of a patient in contact with said viscoelastic foam and prevent or minimize pressure injury to a patient.

2. The system according to claim 1, wherein said single use pad patient support system comprises a single layer of viscoelastic foam configured to make direct contact with a surgical procedure table and direct contact with at least a portion of a patient's skin.

3. The system according to claim 2, wherein said equivalent total frictional characteristic is sufficient to hold a patient on a surgical procedure table at an inclination of at least about 15 degrees.

4. The single use pad patient support system according to claim 3, wherein said pad comprises a tensile strength sufficient to minimize tearing of said pad.

5. The single use pad patient support system according to claim 4, wherein said viscoelastic foam comprises:
a rebound, such as ball rebound, being sufficiently small to minimize undesirable movement of a patient during a medical procedure;
an indentation force deflection sufficient to permit formation of a depression having a depth sufficient to at least assist in holding a patient on a medical procedure table; and
a tensile strength sufficient to minimize tearing of said deformable material.

6. The single use pad patient support system according to claim 5, wherein:
said ball rebound is in the range of approximately 0.1 percent to approximately 5 percent;
said air flow is in the range of approximately 0.1 to approximately 3.0 cubic foot per minute;
said indentation force deflection is in the range of approximately 7 to approximately 18 pounds;
said tensile strength is in the range of approximately 5 pounds per square inch to approximately 15 pounds per square inch; and
said viscoelastic foam further comprises:
a compression set, for a 25 percent compression, of less than approximately one percent; and
a density in the range of approximately 75 kilograms per cubic meter to approximately 110 kilograms per cubic meter.

7. The single use pad patient support system according to claim 6, wherein:
said ball rebound is in the range of approximately 0.1 percent to approximately 1.9 percent;
said air flow is in the range of approximately 0.3 to approximately 1.0 cubic foot per minute;
said indentation force deflection is in the range of approximately 10 to approximately 15 pounds;
said tensile strength is in the range of approximately 8 pounds per square inch to approximately 12 pounds per square inch; and
said viscoelastic foam further comprises:
a compression set, for a 25 percent compression, which is less than approximately 0.3 percent; and
a density in the range of approximately 83 kilograms per cubic meter to approximately 103 kilograms per cubic meter;
said pad is sufficiently thick to permit formation of a depression having portions of different depths, such that the depths of the portions of the depression formed by a buttocks and scapular region of a torso of a patient are greater than the depths of other portions of the depression, and such that the deeper portions of the depression provide a greater portion of the holding forces than shallower portions of the depression;
said single use pad patient support system comprises a retaining arrangement configured to at least assist in the retention of said single use pad patient support system on a surgical procedure table; and
said pad has a thickness in the range of from approximately three-fourths of an inch to three inches or greater.

8. A system configured to minimize injuries on portions of a body of a patient on a surgical procedure table, such as a surgical operating table or a patient examination table, said system comprising a pad;
said pad comprising:
a coefficient of static friction bring sufficient to assist in minimizing unwanted movement of said pad on a surgical procedure table upon a patient being on a surgical procedure table;
sufficient thickness to sufficiently cushion a body of a patient to: minimize bottoming out or prevent bottoming out, on a surgical procedure table, of one or more of portions of a body of a patient during positioning of a patient and during a surgical procedure, and to minimize injuries from pressure on a patient during a surgical procedure; and
sufficient compliance to conform to a substantial portion of a body of a patient;
sufficient thinness to stabilize a patient on a surgical procedure table; and
said sufficient thickness and said sufficient compliance being configured to permit formation of a cavity in said pad of a depth sufficient to:
assist in holding a patient on a surgical procedure table, and
assist in minimizing undesired movement of a body of a patient on a surgical procure table, during a surgical procedure performed while a patient is in a surgical position;
wherein said pad is configured to generate a sufficient static frictional force on skin of a patient on a surgical procedure table pad to stabilize a patient on a surgical procedure table pad;
said system further comprising:
a cleaning device configured to clean a scope during a medical procedure;
said device comprising further:
a casing housing a sponge and a heating element; and
an attachment element connected to an outer surface of said casing and configured to removably attach said casing to a surgical implement;
said surgical implement being configured to be operatively attachable to a patient;
wherein said casing is configured to allow a lens of said scope to access said sponge and said heating element; and
said pad provides an air flow sufficient to provide substantial air flow at at least a portion of a contact area of a patient, and prevent or minimize pressure injury to a patient.

9. A system configured to be used in an operating room, said system comprising:
a removable surgical operating table patient support pad structure configured to support and assist in positioning a patient on a surgical operating table;
said patient support pad comprising a pad comprising a memory foam; and
said system further comprising:
a cleaning device configured to clean a scope during a medical procedure;
said device comprising further:
a casing housing a sponge and a heating element; and
an attachment element connected to an outer surface of said casing and configured to removably attach said casing to a surgical implement;
said surgical implement being configured to be operatively attachable to a patient;
said pad structure yet further comprising:
wherein said pad comprises a thickness to permit deformation of said pad and provide cushioning to assist in positioning a patient stably on a surgical operating table during a medical procedure performed while a patient is in a position,
wherein said memory foam comprises a viscoelastic foam; and said patient support pad further comprises securing straps.
